(19) United States
(12) Patent Application Publication
(10) Pub. No.: US 2025/0186169 A1
(43) Pub. Date: Jun. 12, 2025

(54) OPTIMIZING ORTHODONTIC TREATMENT BASED ON PATIENT PROGRESS

(71) Applicant: Align Technology, Inc., San Jose, CA (US)

(72) Inventors: Christopher E. Cramer, Durham, NC (US); Chad Clayton Brown, Cary, NC (US); Michael Austin Brown, Orlando, FL (US); Rajiv Vankata, San Jose, CA (US); Irina Ivanova, Rotkreutz (CH)

(21) Appl. No.: 18/976,167

(22) Filed: Dec. 10, 2024

Related U.S. Application Data
(60) Provisional application No. 63/608,773, filed on Dec. 11, 2023.

(51) Int. Cl. A61C 7/00; G16H 50/70

(52) U.S. Cl. CPC A61C 7/002; G16H 50/70

(57) ABSTRACT

A method is provided for optimizing a dental treatment plan. In some cases, the method can include receiving patient data comprising one or more progress indicators associated with an dental treatment plan, processing the patient data to determine a level of progression associated with the dental treatment plan based on the one or more progress indicators, modifying the dental treatment plan in response to the determined level of progression, and generating a notification of the modified dental treatment plan. In some cases, modifying the dental treatment plan in response to the determined level of progression includes advancing a patient associated with the patient data to a subsequent stage of the dental treatment plan or retaining the patient within a current stage of the dental treatment plan.

### RELATED APPLICATIONS

This patent application claims the benefit under 35 U.S.C. § 119 (e) of U.S. Provisional Application No. 63/608,773, filed Dec. 11, 2023, which is incorporated by reference herein. This application is related to co-pending U.S. patent application Ser. No. 17/443,244, filed on Jul. 22, 2021, and entitled “Systems, Apparatus, and Methods for Remote Orthodontic Treatment” the contents of which are incorporated by reference in their entirety. This application is additionally related to co-pending U.S. Pat. No. 10,792,127, issued Oct. 6, 2020, and entitled “Adaptive Orthodontic Treatment” the contents of which are also incorporated by reference in their entirety.

### TECHNICAL FIELD

The instant specification generally relates to systems and methods for optimizing a dental treatment plan.

### BACKGROUND

Treatment plans encompass comprehensive outlines of actions aimed at managing medical conditions. They are often developed by healthcare providers in consultation with the patient and typically include diagnostic tests, therapies, and other interventions that are tailored for an individual patient. Treatment plans and strategies function as “roadmaps” for both healthcare providers and the patients, outlining steps and sequence needed to be taken to achieve the desired health outcomes.

### SUMMARY

The below summary is a simplified summary of the disclosure in order to provide a basic understanding of some aspects of the disclosure. This summary is not an extensive overview of the disclosure. It is intended neither to identify key or critical elements of the disclosure, nor delineate any scope of the particular embodiments of the disclosure or any scope of the claims. Its sole purpose is to present some concepts of the disclosure in a simplified form as a prelude to the more detailed description that is presented later.

In some aspects, a method for optimizing a dental treatment plan. In some aspects, the method includes accessing a dental treatment plan for a patient including a series of sequential treatment stages, each treatment stage associated with a particular dental appliance in a series of dental appliances. In some aspects the method further includes receiving patient data including one or more progress indicators associated with the dental treatment plan, determining, based on the one or more progress indicators, a level of progress associated with the dental treatment plan, and based on the determined level of progress, determining a treatment modification for the patient. In some aspects, the treatment modification includes advancing the patient to a subsequent treatment stage in the series of sequential treatment stages before a preplanned advancement time, or retaining the patient in a current stage of the series of sequential treatment stages beyond the preplanned advancement time. In some aspects the method further includes generating a notification indicating the determined treatment modification.

In some aspects, a method for optimizing a dental treatment plan is provided. In some aspects, the method includes that includes receiving patient data including one or more progress indicators associated with a dental treatment plan, processing the patient data to determine a level of progression associated with the dental treatment plan based on the one or more progress indicators, modifying the dental treatment plan in response to the determined level of progression, and generating a notification of the modified dental treatment plan.

In some aspects, a method for optimizing a dental treatment plan is provided. In some aspects, the method includes receiving a plurality of patient data, wherein the plurality of patient data is discretized into data segments associated with individual patients and corresponding individual dental treatment plans for the individual patients, one or more of the data segments including at least one of a three-dimensional (3D) model of a dental arch or a 2D image of the dental arch, and receiving a plurality of dental treatment plan modifications. In some aspects, each dental treatment plan modification of the plurality of dental treatment plan modifications corresponds to a given data segment of the plurality of patient data that is associated with an individual patient and the corresponding individual dental treatment plan for the individual patient. In some aspects the method further includes generating a training dataset including the received plurality of patient data and the received plurality of dental treatment plan modifications. In some aspects, the method further includes training a machine learning model using the training dataset to generate a trained machine learning model trained to output at least one of one or more new dental treatment plans or modifications to one or more existing dental treatment plans based on an input comprising at least one of a 3D model of a new dental arch or a 2D image of the new dental arch.

### INCORPORATION BY REFERENCE

All patents, applications, and publications referred to and identified herein are hereby incorporated by reference in their entirety, and shall be considered fully incorporated by reference even though referred to elsewhere in the application.

### BRIEF DESCRIPTION OF THE DRAWINGS

Aspects and embodiments of the present disclosure will be understood more fully from the detailed description given below and from the accompanying drawings of various aspects and embodiments of the disclosure, which, however, should not be taken to limit the disclosure to the specific aspects or embodiments, but are for explanation and understanding only.

FIG. 1A shows a block diagram of an example system for virtual dental care, in accordance with some embodiments.

FIG. 1B shows a block diagram of an example system for intelligent photo guidance, in accordance with some embodiments.

FIG. 1C shows a block diagram of an example system for image-based assessment, in accordance with some embodiments.

FIG. 1D shows a block diagram of an example system for intelligent patient guidance, in accordance with some embodiments.

FIG. 1E shows a block diagram of an example system for photo-based refinement, in accordance with some embodiments.

FIG. 2A illustrates an example system architecture capable of supporting an optimization module for optimizing a dental treatment plan, in accordance with one embodiment of the present disclosure.

FIG. 2B illustrates an example optimization subprocess of the optimization module of FIG. 2A, according to some embodiments of the present disclosure.

FIG. 2C illustrates an example treatment plan, according to some embodiments of the present disclosure.

FIG. 3 illustrates an example optimization subprocess of the optimization module of FIG. 1, in accordance with some embodiments of the present disclosure.

FIG. 4A illustrates an example data gathering subprocess performed by the data management module of FIG. 1, in accordance with some embodiments of the present disclosure.

FIG. 4B illustrates an example data analysis subprocess performed by the analysis module of FIG. 1, in accordance with some embodiments of the present disclosure.

FIG. 4C illustrates an example AI model of the analysis module of the system of FIG. 1, in accordance with some embodiments of the present disclosure.

FIG. 5A illustrates a flow diagram of an example method for modifying an orthodontic treatment plan, in accordance with some embodiments of the present disclosure.

FIG. 5B illustrates a flow diagram of an example method for modifying an orthodontic treatment plan, in accordance with some embodiments of the present disclosure.

FIG. 5C illustrates a flow diagram of an example method for modifying an orthodontic treatment plan, in accordance with some embodiments of the present disclosure.

FIG. 6 depicts a block diagram of an example processing device operating in accordance with one or more embodiments of the present disclosure.

FIG. 7A illustrates a tooth repositioning appliance, according to certain embodiments.

FIG. 7B illustrates a tooth repositioning system, according to certain embodiments.

FIG. 7C illustrates a method of orthodontic treatment using a plurality of appliances, according to certain embodiments.

FIG. 8 illustrates a method for designing an orthodontic appliance, according to certain embodiments.

FIG. 9 illustrates a method for digitally planning an orthodontic treatment, according to certain embodiments.

FIG. 10A illustrates a series of palatal expanders that are configured to progressively expand the suture, according to certain embodiments.

FIG. 10B illustrates a passive holder (e.g., retainer) that may be worn, for example, after the series of FIG. 10A has completed expanding the patient's palate, according to certain embodiments.

FIG. 11 illustrates a view of multiple treatment timelines in a graphical user interface for a patient system or dental professional device, according to certain embodiments.

### DETAILED DESCRIPTION

A dental treatment plan is a specialized type of treatment plan that focuses on oral health. Created by a healthcare provider (HCP) such as a dentist or dental specialist, a dental treatment plan outlines the procedures and sequence of operations or treatments needed to address issues such as misaligned teeth, a narrow palate, gum disease, malocclusions, caries, gum recession, and other oral health issues. Dental treatment plans may include restorative treatment plans, palatal expansion treatment plans and/or orthodontic treatment plans in some embodiments. In some cases, combined treatment plans may be determined that combine two or more of restorative dental treatment, palatal expansion treatment and orthodontic treatment. By providing a structured approach to dental treatment, a dental treatment plan helps both the healthcare provider and the patient understand and execute the necessary steps for achieving oral health goals.

Dental treatment plans often include multi-stage plans that are broken down into various phases or stages. Each phase has its own set of specific procedures and goals, which are designed to address different aspects of the dental issue at hand. For example, an orthodontic treatment plan may start with an initial phase focused on aligning the front teeth, followed by a second phase aimed at perfecting the bite. Similarly, a palatal expansion treatment plan may start with an initial phase focused on expanding the palate, followed by a second phase aimed at retaining the palate in the expanded state. A combined palatal expansion and orthodontic treatment plan may start with an initial phase focused on expanding the palate, followed by a second phase aimed at retaining the palate in the expanded state, followed by a third phase focused on correcting one or more malocclusions by repositioning the patient's teeth. These phases can be further granularized to enhance precision and targeting in the treatment plan.

Such a phased dental plan can further include scheduled stages or checkpoints at timepoints within, or between, each phase. Such checkpoints are junctures where a dental HCP evaluates the progress of the treatment and decides whether it is appropriate to move on to the subsequent phase. Regularly scheduled checkpoints provide an opportunity for re-evaluation and, if necessary, modification of the treatment plan. This ensures that the treatment is on the right track and allows for course corrections, enhancing the likelihood of a successful outcome.

Treatment plans, types, stages, and checkpoints can vary widely in scope and duration, due to individual patient differences. Factors such as biology, tooth structure, age, genetics, diet, emerging conditions, etc., can heavily influence effectiveness and/or progression of a treatment plan for a specific individual. For example, younger patients may require different treatment modalities compared to older individuals, genetic factors can influence how quickly a patient responds to treatment, etc.

Such idiosyncrasies can present a challenge by demanding a flexible approach that strikes a balance between the structure and scheduling required for progression, while maintaining the versatility and flexibility to accommodate individualized factors and emergent conditions that arise during treatment. Designing, implementing, monitoring, and updating such a treatment plan can be challenging.

In light of these complexities, ongoing patient assessment and treatment plan adjustment are beneficial elements of any dental treatment plan. Accordingly, deepening levels of personalization, and decreased levels of lifestyle intrusiveness by a treatment plan can provide added benefits by addressing each patient's unique health goals and biology.

In embodiments, a system may optimize a dental treatment plan. The system may access a dental treatment plan for a patient comprising a series of sequential treatment stages, each treatment stage associated with a particular dental appliance in a series of dental appliances. The dental appliance may be an orthodontic aligner, wires and brackets, a palatal expander, a dental appliance for delivering a tooth whitening agent, or some other type of dental appliance. The system may receive patient data comprising one or more progress indicators (and/or processable to determine one or more progress indicators) associated with the dental treatment plan. The patient data may include, for example, images of a patient's dentition (e.g., from an intraoral scanner; a smartphone or other camera device), biomarker data indicative of changes in the patient's dentition, pressure data indicative of a level of pressure exerted by the patient's dentition on an orthodontic aligner, and/or data indicative of an electrical parameter associated with a position of a tooth with respect to an orthodontic aligner, and so on.

The system may determine, based on the one or more progress indicators, a level of progress associated with the dental treatment plan. Based on the determined level of progress, the system may determine a treatment modification for the patient. The system may decide to advance the patient to a subsequent treatment stage in the series of sequential treatment stages before a preplanned advancement time, or to retain the patient in a current stage of the series of sequential treatment stages beyond a preplanned advancement time. The system may generate a notification indicating the determined treatment modification.

Embodiments enable a patient to progress through a treatment plan faster than initially planned or slower than initially planned based on patient data collected during implementation of the treatment plan. This may enable treatment speeds to be tailored to the patient based on the patient's compliance with the treatment plan (e.g., how much time the patient wears their aligners), the patient's physiology, and so on.

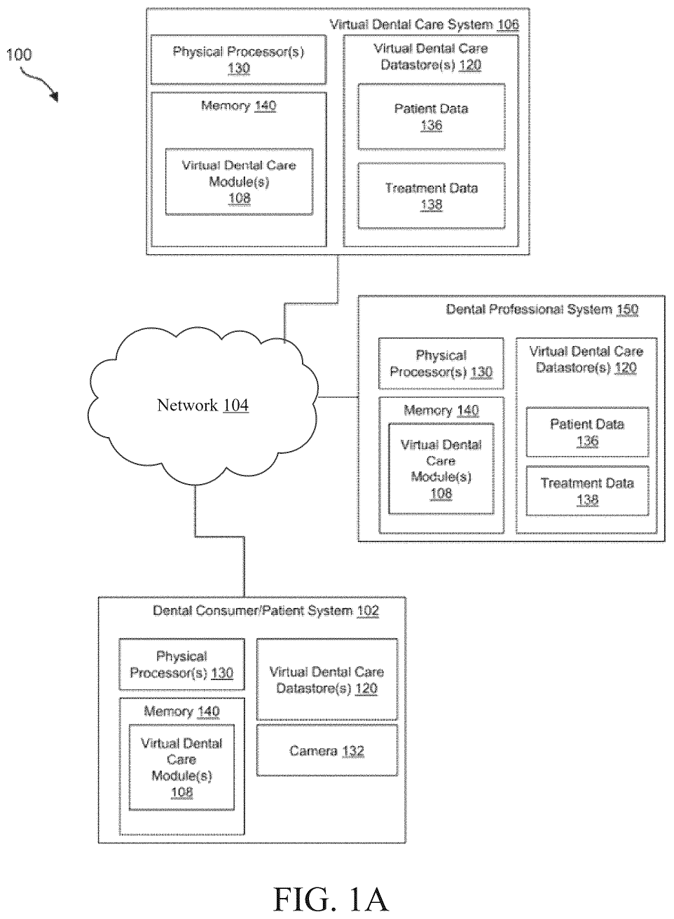

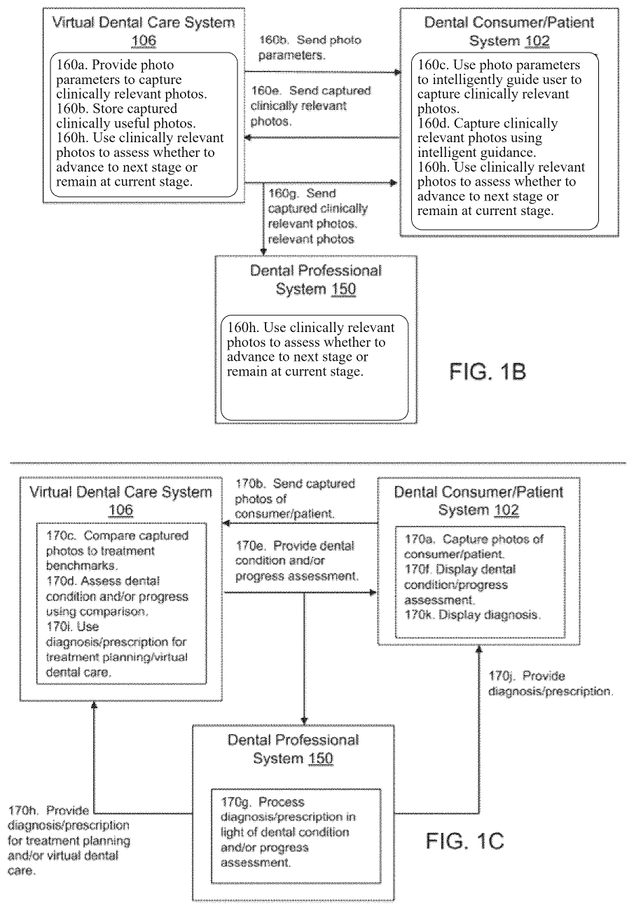

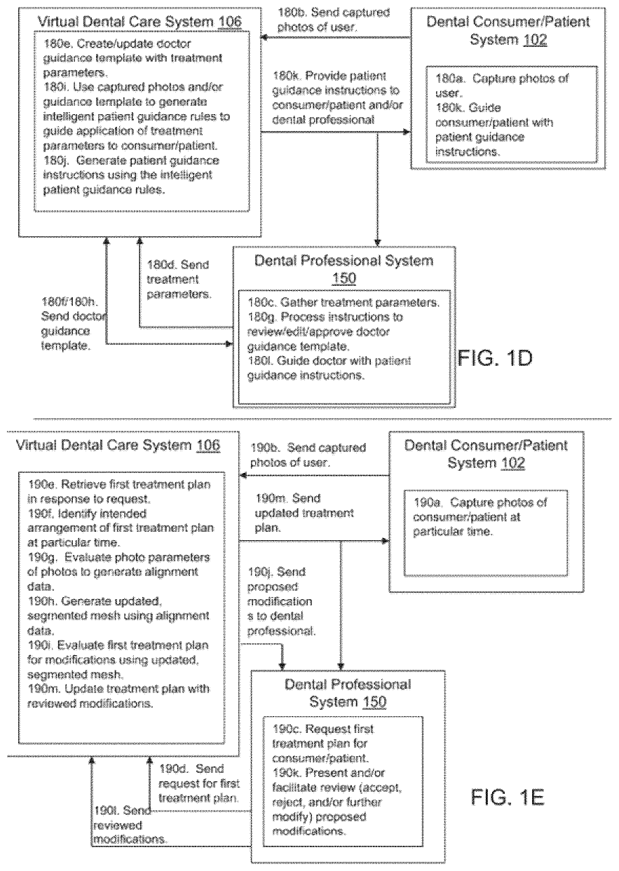

Thus, aspects and implementations of the present disclosure address the above challenges of dental treatment plans by providing systems and methods to further personalize and optimize a dental treatment plan, while limiting lifestyle invasiveness. The proposed systems and methods provided herein may include an optimization module for analyzing and quantifying a patient's treatment progress, and optimizing the stages of a treatment plan, according to some embodiments of the present disclosure. For instance, in some embodiments, an initial treatment plan may be generated (e.g., based on patient information such as patient demographics, type of treatment required, a patient's particular teeth arrangement, data gathered from a dental professional system used to scan the patient, etc.), after a patient has visited a dentist and/or orthodontist office. One or more dental devices (e.g., aligners, palatal expanders, retainers, etc.) may be made for the patient, in accordance with scheduled stages and checkpoints of the generated, initial treatment plan. As the treatment plan is on-going, the patient may use an at-home scanner (or a smartphone, or a similar data collection device, etc.), to monitor progress at predetermined checkpoints determined in the initial treatment plan. Data may thus be collected at the checkpoints. For example, images of teeth captured at various views may be captured to determine teeth movement, patient compliance data may be captured by sensors that monitor how long the device is being worn, and so on. Afterwards, the optimization system and/or module may determine that the initial treatment plan may need to be updated based on the data. For example, the patient may be instructed to stay at the current stage for a specified number of days, the patient may be advanced earlier than scheduled to a subsequent stage, the checkpoint intervals may be updated, and/or a similar modification to the initial plan may be made. Furthermore, based on the captured data, more or less monitoring may be determined as necessary. For example, more monitoring may be planned in cases where a patient is less compliant, more monitoring may be planned for a patient who appears to be presenting with some oral health risk (e.g., gingival recession, potential occlusion issues, potential tooth root fenestration), more monitoring may be planned for a patient whose teeth aren't moving according to plan, etc. Thus, an updated plan and/or updated checkpoint intervals may be generated based on collected data and sent to the patient. Thus, a patient may receive and follow an updated plan including updated checkpoint intervals. Such a system will be illustratively described throughout the specification set forth below (i.e., with respect to FIGS. 1A-10B). Furthermore, exemplary embodiments of such a system will follow throughout the specification set forth below (i.e., with respect to FIGS. 1A-10B).

Embodiments described herein provide significant advantages over traditional dental treatment plans, techniques, and orthodontics. Such embodiments may provide a system that notifies a health care provider (HCP) and/or patient of a level of progression with respect to a dental treatment plan. Such embodiments may provide a system that provides modifications to the dental treatment plan based on the level of progression and suggest actions to perform to ensure that a planned treatment outcome is achieved. Accordingly, treatment plan efficacy is improved and optimized, in embodiments. Such improvements in treatment plan efficacy are likely to result in increased patient satisfaction as well as reduced costs by reducing the number of consecutive refinements that are made to a treatment plan (and associated orders of additional aligners) during treatment.

Embodiments are discussed herein with reference to multi-stage treatment plans. However, such embodiments also apply to single stage treatment plans that have a target end position. For example, image data may be generated some time after beginning a single stage orthodontic treatment plan. If the image data shows that progress of the single stage treatment plan is not as expected, then the target end position may be adjusted for the single stage treatment plan and/or one or more treatment parameters for reaching the target end position may be adjusted. Accordingly, it should be understood that all discussion of multi-stage treatment plans herein also applied to single stage treatment plans with target end positions and/or conditions.

Furthermore, some embodiments are discussed herein with reference to dental treatment, such as orthodontic treatment and orthorestorative treatment (e.g., where an orthodontic treatment is performed in a first phase, and restorative treatment such as a crown or veneer is performed in a subsequent second phase; or vice versa). However, it should be understood that embodiments discussed with reference to dental treatment plans also apply to other medical treatment plans, such as other types of multi-stage medical treatment plans where there are multiple stages that require some active step and/or monitoring (e.g., by the patient, by an HCP, by an automated system) to advance to another (e.g., subsequent) stage.

Furthermore, some embodiments are discussed herein with reference to orthodontic treatment plans that include generation and use of orthodontic aligners (also referred to simply as aligners). As used herein, an aligner is an orthodontic appliance that is used to reposition teeth. In some embodiments, orthodontic appliances, such as aligners, impart forces to the crown of a tooth and/or a dental auxiliary (e.g., an attachment) positioned on the tooth at one or more points of contact between a tooth receiving cavity of the appliance and received tooth and/or dental auxiliary. The magnitude of each of these forces and/or their distribution on the surface of the tooth can determine the type of orthodontic tooth movement which results.

Tooth movements may be in any direction in any plane of space, and may comprise one or more of rotation or translation along one or more axes. Types of tooth movements include extrusion, intrusion, rotation, tipping, translation, and root movement, and combinations thereof, as discussed further herein. Tooth movement of the crown greater than the movement of the root can be referred to as tipping. Equivalent movement of the crown and root can be referred to as translation. Movement of the root greater than the crown can be referred to as root movement.

It should be noted that embodiments also apply to other types of dental treatment that may incorporate use of one or more other dental and/or orthodontic appliances including but not limited to brackets and wires, retainers, palatal expanders, and/or other functional appliances. Accordingly, it should be understood that any discussion of aligners herein also applies to other types of orthodontic and/or dental appliances.

FIG. 1A shows a block diagram of an example system for virtual dental care, in accordance with some embodiments. As shown in FIG. 1A, system 100 may include a dental consumer/patient system 102, a dental professional system 150, a virtual dental care system 106, and a network 104. The dental consumer/patient system 102, dental professional system 150, and virtual dental care system 106 may communicate to one another over the network 104, which may include one or more local area networks (LANs), public wide area networks (e.g., such as the Internet) and/or private wide area networks (e.g., Intranets), one or more personal Area Networks (PAN), one or more cellular networks (e.g., a Global System for Mobile Communications (GSM) network), and/or any other suitable network.

Dental consumer/patient system 102 generally represents any type or form of computing device capable of reading computer-executable instructions. Dental consumer/patient system 102 may be, for example, a desktop computer, a tablet computing device, a laptop, a smartphone, an augmented reality device, or other consumer device. Additional examples of dental consumer/patient system 102 include, without limitation, laptops, tablets, desktops, servers, cellular phones, Personal Digital Assistants (PDAs), multimedia players, embedded systems, wearable devices (e.g., smart watches, smart glasses, etc.), smart vehicles, smart packaging (e.g., active or intelligent packaging), gaming consoles, Internet-of-Things devices (e.g., smart appliances, etc.), variations or combinations of one or more of the same, and/or any other suitable computing device. The dental consumer/patient system 102 need not be or include a clinical scanner (e.g., an intraoral scanner), though it is contemplated that in some implementations the functionalities described herein in relation to the dental consumer/patient system 102 may be incorporated into a clinical scanner. As an example of various implementations, a camera 132 of the dental consumer/patient system 102 may comprise an ordinary camera that captures 2D images of the patient's dentition and does not capture height-map and/or other data (e.g., three-dimensional (3D) data) that is used to stitch a mesh of a 3D surface. In some examples, the dental consumer/patient system 102 may include an at-home intraoral scanner.

In some implementations, the dental consumer/patient system 102 is configured to interface with a dental consumer and/or dental patient. A “dental consumer,” as used herein, may include a person seeking assessment, diagnosis, and/or treatment for a dental condition (general dental condition, orthodontic condition, endodontic condition, condition requiring restorative dentistry, etc.). A dental consumer may, but need not, have agreed to and/or started treatment for a dental condition. A “dental patient,” as used herein, may include a person who has agreed to diagnosis and/or treatment for a dental condition. A dental consumer and/or a dental patient, may, for instance, be interested in and/or have started orthodontic treatment, such as treatment using one or more (e.g., a sequence of) aligners (e.g., polymeric appliances having a plurality of tooth-receiving cavities shaped to successively reposition a person's teeth from an initial arrangement toward a target arrangement). In various implementations, the dental consumer/patient system 102 provides a dental consumer/dental patient with software (e.g., one or more webpages, standalone applications, mobile applications, etc.) that allows the dental consumer/patient to capture images of their dentition, interact with dental professionals (e.g., users of the dental professional system 150), manage treatment plans (e.g., those from the virtual dental care system 106 and/or the dental professional system 150), and/or communicate with dental professionals (e.g., users of the dental professional system 150).

Dental professional system 150 generally represents any type or form of computing device capable of reading computer-executable instructions. Dental professional system 150 may be, for example, a desktop computer, a tablet computing device, a laptop, a smartphone, an augmented reality device, or other consumer device. Additional examples of dental professional system 150 include, without limitation, laptops, tablets, desktops, servers, cellular phones, Personal Digital Assistants (PDAs), multimedia players, embedded systems, wearable devices (e.g., smart watches, smart glasses, etc.), smart vehicles, smart packaging (e.g., active or intelligent packaging), gaming consoles, Internet-of-Things devices (e.g., smart appliances, etc.), variations or combinations of one or more of the same, and/or any other suitable computing device.

In various implementations, the dental professional system 150 is configured to interface with a dental professional. A “dental professional” (used interchangeably with dentist, orthodontist, and doctor herein) as used herein, may include any person with specialized training in the field of dentistry, and may include, without limitation, general practice dentists, orthodontists, dental technicians, dental hygienists, etc. A dental professional may include a person who can assess, diagnose, and/or treat a dental condition. “Assessment” of a dental condition, as used herein, may include an estimation of the existence of a dental condition. An assessment of a dental condition need not be a clinical diagnosis of the dental condition. In some embodiments, an “assessment” of a dental condition may include an “image based assessment,” that is an assessment of a dental condition based in part or on whole on photos and/or images (e.g., images that are not used to stitch a mesh or form the basis of a clinical scan) taken of the dental condition. A “diagnosis” of a dental condition, as used herein, may include a clinical identification of the nature of an illness or other problem by examination of the symptoms. “Treatment” of a dental condition, as used herein, may include prescription and/or administration of care to address the dental conditions. Examples of treatments to dental conditions include prescription and/or administration of brackets/wires, clear aligners, palatal expanders, and/or other appliances to address orthodontic conditions, prescription and/or administration of restorative elements to address bring dentition to functional and/or aesthetic requirements, etc. The dental professional system 150 may provide to a user software (e.g., one or more webpages, standalone applications (e.g., dedicated treatment planning and/or treatment visualization applications), mobile applications, etc.) that allows the user to interact with users (e.g., users of the dental consumer/patient system 102, other dental professionals, etc.), create/modify/manage treatment plans (e.g., those from the virtual dental care system 106 and/or those generated at the dental professional system 150), etc.

Virtual dental care system 106 generally represents any type or form of computing device that is capable of storing and analyzing data. Virtual dental care system 106 may include a backend database server for storing patient data and treatment data. Additional examples of virtual dental care system 106 include, without limitation, security servers, application servers, web servers, storage servers, and/or database servers configured to run certain software applications and/or provide various security, web, storage, and/or database services. Although illustrated as a single entity in FIG. 1A, virtual dental care system 106 may include and/or represent a plurality of servers that work and/or operate in conjunction with one another.

As illustrated in FIG. 1A, dental consumer/patient system 102, virtual dental care system, 106, and/or dental professional system 150 may include one or more memory devices, such as memory 140. Memory 140 generally represents any type or form of volatile or non-volatile storage device or medium capable of storing data and/or computer-readable instructions. In one example, memory 140 may store, load, execute in conjunction with physical processor(s) 130, and/or maintain one or more of virtual dental care modules 108. Examples of memory 140 include, without limitation, Random Access Memory (RAM), Read Only Memory (ROM), flash memory, Hard Disk Drives (HDDs), Solid-State Drives (SSDs), optical disk drives, caches, variations, or combinations of one or more of the same, and/or any other suitable storage memory.

As illustrated in FIG. 1A, dental consumer/patient system 102, dental professional system 150, and/or virtual dental care system 106 may also include one or more physical processors, such as physical processor(s) 130. Physical processor(s) 130 generally represents any type or form of hardware-implemented processing unit capable of interpreting and/or executing computer-readable instructions. In one example, physical processor(s) 130 may access and/or modify one or more of virtual dental care modules 108 stored in memory 140. Additionally or alternatively, physical processor 130 may execute one or more of virtual dental care modules 108 to facilitate preamble phrase. Examples of physical processor(s) 130 include, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, and/or any other suitable physical processor.

In some embodiments, dental consumer/patient system 102 may include a camera 132. Camera 132 may comprise a camera, scanner, or other optical sensor. Camera 132 may include one or more lenses or may, one or more camera devices, and/or one or more other optical sensors. In some examples, camera 132 may include other sensors and/or devices which may aid in capturing optical data, such as one or more lights, depth sensors, etc. In various implementations, the camera 132 is not a clinical scanner.

Virtual dental care datastore(s) 120 include one or more datastore configured to store any type or form of data that may be used for virtual dental care. In some embodiments, the virtual dental care datastore(s) 120 include, without limitation, patient data 136 and treatment data 138. Patient data 136 may include data collected from patients, such as patient dentition information, patient historical data, patient scans, patient information, etc. Treatment data 138 may include data used for treating patients, such as treatment plans, state of treatment, success of treatment, changes to treatment, notes regarding treatment, etc.

As will be described in greater detail below, one or more of virtual dental care modules 108 and/or the virtual dental care datastore(s) 120 in FIG. 1A may, (when executed by at least one processor of dental consumer/patient system 102, virtual dental care system 106, and/or dental professional system 150) enable dental consumer/patient system 102, virtual dental care system 106, and/or dental professional system 150 to optimize dental treatment of an existing dental treatment plan and/or generate a new dental treatment plan.

Some embodiments provide patients with “Virtual dental care.” “Virtual dental care,” as used herein, may include computer-program instructions and/or software operative to provide remote dental services by a health professional (dentist, orthodontist, dental technician, etc.) to a patient, a potential consumer of dental services, and/or other individual. Virtual dental care may comprise computer-program instructions and/or software operative to provide dental services without a physical meeting and/or with only a limited physical meeting. As an example, virtual dental care may include software operative to providing dental care from the dental professional system 150 and/or the virtual dental care system 106 to the computing device 102 over the network 104 through e.g., written instructions, interactive applications that allow the health professional and patient/consumer to interact with one another, telephone, chat etc. Some embodiments provide patients with “Remote dental care.” “Remote dental care,” as used herein, may comprise computer-program instructions and/or software operative to provide a remote service in which a health professional provides a patient with dental health care solutions and/or services. In some embodiments, the virtual dental care facilitated by the elements of the system 100 may include non-clinical dental services, such as dental administration services, dental training services, dental education services, etc.

In some embodiments, the elements of the system 100 (e.g., the virtual dental care modules 108 and/or the virtual dental care datastore(s) 120) may be operative to provide intelligent photo guidance to a patient to take images relevant to virtual dental care using the camera 132 on the computing device 102. An example of how the elements of the system 100 may operate to provide intelligent photo guidance is shown in FIG. 1B.

At an operation 160a, the virtual dental care system 106 may provide one or more photo parameters to capture clinically relevant photos of a user. “Clinically relevant” photos, as used herein, may include images that represent the state of dental conditions in a consumer/patient's dentition. Clinically relevant photos may include photos that are sufficient to provide current position(s) and/or orientation(s) of the teeth in a consumer/patient's mouth. Examples of clinically relevant photos include photos that show all the teeth in a consumer/patient's arch; photos that show the shape of a consumer/patient's arch; photos that show locations of teeth that are missing, supernumerary, ectopic, etc.; photos that show malocclusions in a consumer/patient's arch (e.g., from front, left buccal, right buccal, and/or other various perspectives); etc. “Photo parameters,” as used this context, may include parameters to define clinically acceptable criteria (e.g., clinically acceptable position(s) and/or clinically acceptable orientation(s) of teeth) in one or more photos. In embodiments, some photos may show a patient's dentition and a dental appliance worn over the patient's dentition, while some photos may show the patient's dentition without a dental appliance. Photo parameters can include a distance parameters, e.g., one that parametrizes a distance that a camera is relative to a consumer/patient's dentition; orientation parameters (e.g., those that parametrize orientations of photos taken of teeth); openness parameters of a photo of a consumer/patient's bite (e.g., whether a bite is open, closed, and/or a degree of openness of a bite); a dental appliance wear parameter of a photo of a consumer/patient's bite (e.g., whether a photo shows dental appliances, such as cheek retractors, aligners, etc. in a consumer/patient's mouth); camera parameters (brightness parameters of photos; contrast parameters of photos; exposure parameters of photos; etc.); tooth identifier parameters, e.g., those that parametrize the specific teeth in a photo, those taken from a treatment plan; etc. At an operation 160b, the virtual care dental system 106 may send the one or more photo parameters to the dental consumer/patient system 102. This operation can occur as a file and/or data transfer over the computer-readable medium 104.

At an operation 160c, the dental consumer/patient system 102 may use the one or more photo parameters to intelligently guide the consumer/patient to capture clinically relevant photos of their dentition. The dental consumer/patient system 102 may gather image-capture rules that guide capturing the clinically relevant photos based on the photo parameters. The dental consumer/patient system 102 may provide a consumer/patient with software (e.g., one or more webpages, standalone applications, mobile applications, etc.) that uses the one or more photo parameters to help the consumer/patient capture clinically relevant photos of their teeth. As an example, distance parameters may be used to guide a consumer/patient to position and/or orient the dental consumer/patient system 102a specific distance away from their teeth to capture a photo with appropriate details of their teeth. The distance parameters may guide whether the position of a camera is too close or too far or just right. Orientation parameters may be used to guide a photo to clinically relevant orientations. As an example, orientation parameters may be used to guide a consumer/patient to take photos of anterior views, left buccal views, right buccal views, etc. As additional examples, openness parameters may be used to guide a consumer/patient to take photos of various bite states, e.g., an open bite, closed bite, and/or a bite that is partially open in order to be clinically relevant; dental appliance wear parameters may be used to detect cheek retractors and/or guide a consumer/patient to position cheek retractors appropriately and/or locate/orient photos to be clinically relevant; dental appliance wear parameters may be used to detect various dental appliances (aligners, retainers, etc.) and guide a consumer to remove, move, etc. the dental appliances for photos that are clinically relevant; etc. Additionally, tooth identifier parameters (e.g., those gathered from a treatment plan) can be used to guide a consumer/patient to take photos of a sufficient number of teeth so that the photos are clinically relevant. Camera parameters, e.g., contrast, brightness, exposure, etc. parameters may be used to guide consumers/patients to take photos that have properties such that the photos are clinically relevant. In some implementations, the dental consumer/patient system 102 uses camera parameters to modify one or more photo settings (add/disable flash, adjust zoom, adjust brightness, adjust contrast, adjust shadows, adjust silhouettes, etc. so that clinically relevant photos are captured under various conditions. As noted herein, the operation 160c may be performed by automated agents and without human intervention.

At an operation 160d, the dental consumer/patient system 102 may operate to capture clinically relevant photos using the intelligent guidance. In some implementations, a consumer/patient may follow instructions to capture photos of their dentition using the intelligent guidance provided on the dental consumer/patient system 102. In various implementations, at least a part of operation 160d is performed by automated agents that configure a camera to take photos without human intervention. At an operation 160e, the dental consumer/patient system 102 may send captured clinically relevant images to the virtual dental care system 106. This operation may occur as a file and/or data transfer over the computer-readable medium 104.

At an operation 160f, the virtual dental care system 106 may store the captured clinically relevant photos. In various implementations, the virtual dental care system 106 may store the captured clinically relevant photos in a treatment database associated with a consumer/patient, a clinical data file associated with a consumer/patient, and/or in any relevant datastore. At an operation 160g, the virtual dental care system 106 may send captured clinically relevant photos to the dental consumer/patient system 102 and/or the dental professional system 150. This operation may occur over a file and/or data transfer over the computer-readable medium 104.

At an operation 160h, the dental consumer/patient system 102, the virtual dental care system 106 and/or the dental professional system 150 may use clinically relevant photos to assess patient progress with regards to a dental treatment plan. As an example, the dental professional system 150 may process the clinically relevant photos to assess whether to advance to a subsequent stage of a dental treatment earlier than indicated by a treatment plan or to remain in a current stage of dental treatment longer than indicated by the treatment plan. In some implementations, the dental consumer/patient system 102, the virtual dental care system 106 and/or the dental professional system 150 may, e.g., use clinically relevant photos for image-based assessments, intelligent patient guidance, and/or photo-based refinements. The dental consumer/patient system 102, the virtual dental care system 106 and/or the dental professional system 150 may additionally or alternatively make other determinations with respect to a dental treatment plan and/or with respect to a patient's dentition based on analysis of the clinically relevant photos in embodiments. For example, dental consumer/patient system 102, the virtual dental care system 106 and/or the dental professional system 150 may identify one or more oral health conditions and/or a severity of such identified oral health conditions. In another example, dental consumer/patient system 102, the virtual dental care system 106 and/or the dental professional system 150 may identify missing dental auxiliaries (e.g., missing attachments). As used herein, a dental auxiliary may be any object that is affixed to one or more teeth and that is designed to engage a dental appliance (e.g., an aligner, attachment template, palatal expander, retainer, mouth guard, etc.) to facilitate an orthodontic treatment or to maintain/protect teeth. Examples of dental auxiliaries include dental attachments, buttons, power arms, brackets, and so on.

In another example, dental consumer/patient system 102, the virtual dental care system 106 and/or the dental professional system 150 may determine a level of wear on a dental appliance (e.g., on retainer). In another example, dental consumer/patient system 102, the virtual dental care system 106 and/or the dental professional system 150 may identify one or more teeth that have relapsed in position after palatal expansion treatment and/or orthodontic treatment.

Based on the determination about patient progress with respect to the dental treatment plan, about the patient's dentition, etc., the dental consumer/patient system 102, the virtual dental care system 106 and/or the dental professional system 150 may determine one or more actions to perform, and may perform the one or more actions and/or direct the doctor, patient and/or other devices to perform the one or more actions. Examples of such actions include determining a treatment modification, generating a notification, outputting visualizations, increasing or decreasing a frequency with which photos of the patient's dentition are captured during the dental treatment plan, adjusting an assessment schedule, addressing one or more identified oral health conditions (e.g., by recommending restorative treatment), outputting patient recommendations, outputting doctor recommendation, scheduling an in-person doctor visit for a patient, recommending replacing one or more missing dental auxiliaries, ordering one or more dental appliances, simulating predicted future smile/faces/dentition of a patient, and so on.

In some embodiments, the elements of the system 100 (e.g., the virtual dental care modules 108 and/or the virtual dental care datastore(s) 120) may be operative to provide one or more image-based assessment tools to the users of the dental professional system 150. “Image based assessment tools,” as used herein, may include digital tools that operate to provide image-based assessments of a dental condition. Such image-based assessments may be assessments of a dentition currently undergoing treatment and/or of a dentition for which treatment has not yet begun and/or for which treatment has been completed in embodiments. In some embodiments, image-based assessments may comprise visualizations that allow a user of the dental professional system 150 to make a decision about a clinical condition and/or progress of a treatment plan. As noted herein, visualizations may include, e.g., visualizations of assessments of a current stage of a treatment plan; visualizations of assessments may, but need not, be based on images and knowledge of a treatment plan that is underway. As another example, the elements of the system 100 may provide visualizations to a user of the dental professional system 150 that provide a view of a patient's assessment over time. An example of how the elements of the system 100 may operate to provide image-based assessment tools is shown in FIG. 1C.

At an operation 170a, the dental consumer/patient system 102 may capture one or more images of a consumer/patient. The one or more images may comprise photos taken by the camera of the dental consumer/patient system 102. The one or more photos may be captured by intelligent photo guidance techniques described further herein. The one or more images may include various perspectives and/or views of the dentition of the consumer/patient. The one or more photos captured at operation 170a need not include scan data, height map information, and/or data a clinical scanner uses to stitch together a mesh representation of consumer/patient's dentition. The dental consumer/patient system 102 may store images captured locally, in a networked folder, etc. At an operation 170b, the dental consumer/patient system 102 may send captured photos of the consumer/patient to the virtual dental care system 106. This operation may include a file and/or other data transfer over the computer-readable medium 104.

At an operation 170c, the virtual dental care system 106 may compare the captured photos to one or more treatment benchmarks. “Treatment benchmarks,” as used herein, may include one or more standards or reference points of at least part of a treatment plan. Treatment benchmarks may include intended positions of teeth, jaws, palatal regions, etc. of dentition at a specific stage of a treatment plan. In some implementations, treatment benchmarks are represented as intended positions of a specific stage of a treatment plan on a 3D model of a patient's dentition. In various implementations, treatment benchmarks correspond to representations of a patient's dentition from which to assess a dental condition. As examples, treatment benchmarks may represent a variety of malocclusions for which the consumer/patient is to be assessed. At an operation 170d, the virtual care dental system 106 may assess a dental condition and/or progress of a treatment plan using the comparison of the captured photos and the treatment benchmarks. As noted herein, the assessment need not comprise a diagnosis of the dental condition and/or the progress through the treatment plan.

At an operation 170e, the virtual dental care system 106 may provide the dental consumer/patient system 102 and/or the dental professional system 150 the assessed dental condition and/or the progress assessment. The progress assessment may include an assessment as to whether the patient's dentition is responding to treatment as planned, is responding to treatment faster than planned, is responding to treatment slower than planned, and so on. This operation may occur as a file and/or data transfer over the computer-readable medium 104. The dental consumer/patient system 102 and/or the dental professional system 150 may perform additional operations with the assessed dental condition and/or the progress assessment. As one example, the dental consumer/patient system 102 may, at an operation 170f, display the dental condition and/or the progress assessment. For instance, the dental consumer/patient system 102 may display, e.g., in an application and/or in webpages, user interface elements (annotated 3D models, annotated images, informative and/or interactive user interface elements, etc.) that show an assessment to a consumer/patient.

As another example, the dental professional system 150 may, in an operation 170g, process a diagnosis and/or prescription for a consumer/patient using the dental condition and/or progress assessment. In the operation 170g, the diagnosis may also be based on one or more clinical images (intraoral scans, x-rays, CBCT scans, etc.) of the consumer/patient's dentition. In some implementations, a doctor may use software on the dental professional system 150 to perform a diagnosis of a dental condition and/or of progress of a treatment plan. As an example, a doctor may use treatment planning software on the dental professional system 150 to diagnose malocclusions and/or other dental conditions reflected in the photos from the consumer/patient. Instructions corresponding to the diagnosis may be processed by the dental professional system 150. In various implementations, a dental professional may provide a prescription to treat one or more dental conditions. As an example, a dental professional may prescribe through the dental professional system 150 one or more dental appliances (clear aligners, orthodontic appliances, restorative appliances, palatal expanders, etc.) to treat dental conditions that are associated with the dental condition and/or progress assessment. For an initial assessment, the prescription may comprise an initial prescription for dental appliances. For a progress assessment, the prescription may comprise corrective dental appliances that are configured to correct deviation(s) from a treatment plan. For a progress assessment, the prescription may include a modification to the treatment plan to cause a patient to remain in a stage of treatment for longer than originally planned, or a modification to the treatment plan to cause the patient to progress to a subsequent stage of treatment prior than originally planned.

At an operation 170h, the dental professional system 150 may provide the diagnosis and/or prescription for treatment planning and/or virtual dental care to the virtual dental care system 106. At an operation 170i, the virtual care dental system 106 may use the diagnosis/prescription for treatment planning and/or virtual dental care. At an operation 170j, the dental professional system 150 may provide the diagnosis and/or prescription to the dental consumer/patient system 102. At an operation 170k, the dental consumer/patient system 102 may display the diagnosis and/or updated treatment plan to the consumer/patient.

In some embodiments, the elements of the system 100 (e.g., the virtual dental care modules 108 and/or the virtual dental care datastore(s) 120) may be operative to provide intelligent patient guidance to consumers/patients that use the dental consumer/patient system 102. “Intelligent patient guidance,” as used herein, may include instructions to guide a consumer/patient to take one or more actions. In some implementations, the elements of the system 100 generate intelligent patient guidance using photos of a consumer/patient, treatment parameters supplied by a doctor, instructions to continue using a current aligner or other dental appliance or to switch to a new aligner or dental appliance associated with a subsequent treatment stage and/or other information.

In some implementations, intelligent patient guidance is supplied by automated agents without intervention (or with minimal intervention, e.g., a doctor providing treatment parameters and/or interacting with a guidance template). Intelligent patient guidance may include: e.g., instructions to change (and/or when to change) a specific dental appliance (e.g., an aligner, a retainer, etc.); instructions to continue use (and/or when to continue use) of a dental appliance in relation to a subsequent dental appliance, instructions to use (and/or a location of use) of a supplemental dental appliance (e.g., chewie, mint, etc.); instructions to direct attention to a region of a consumer/patient's dentition (anterior portions, posterior portions, portions that are likely to move during a specific stage, portions that anchor various tooth movements, etc.); instructions to notify a doctor at a specific time or in response to a specific event (e.g., teeth moving at a specific time, teeth moving in accordance with a specific movement pattern, etc.); instructions to capture one or more images of a consumer/patient's dentition for the purpose of progress tracking at a specified time/treatment stage; instructions to the consumer/patient to visit a doctor, set an appointment, or take other action in relation to a doctor; instructions to the consumer/patient to be more compliant in wearing the dental appliances, etc. As noted herein, intelligent patient guidance can include any combination and/or variations of the foregoing examples.

Intelligent patient guidance supplied by the elements of the system 100 may be based on a dental condition and/or progress assessment (e.g., one reflected by images captured by a consumer/patient), treatment parameters, etc. “Treatment parameters,” as used herein, may include a set of parameters that are used to specify attributes of a treatment plan to apply to a consumer/patient. Treatment parameters may include doctor-preference parameters, e.g., treatment parameters specifying treatment protocols that a doctor (and/or other doctors, e.g., those whose treatment protocols are used by a specific doctor) would prescribe for various patients and/or clinical conditions. Treatment parameters may include per-patient parameters, e.g., parameters used to specify treatment protocols for a specific consumer/patient. Per-patient parameters may be based on attributes of a consumer/patient (past treatments, anatomical information (attributes of specific dentitions, jaws, etc.), etc. Per-patient parameters need not be based on attributes of a specific consumer/patient, and, e.g., may include demographic information (information related to the consumer/patient's race, gender, age, etc.), information about historically treated cases (e.g., those with similar forms of dental conditions to the consumer/patient) information about idealized dental arches (e.g., those related to dental arches with idealized/near-idealized occlusions as defined by treatment professionals), and/or other information.

In some implementations, the elements of the system 100 may utilize a doctor guidance template, which, as used herein, may include a formatted data structure that specifies a set of rules that a doctor can use for tracking a treatment plan. Examples of rules could be as specific as central incisors deviations from the treatment plan of 0.75 millimeters (mm) should result in a new appointment; central incisor deviations of 0.5-0.75 mm should be watched; central incisor deviations that increase over a period of two (2) months should result in a new appointment; central incisor deviations of 0.25 to 0.5 mm should wear the current set of aligners for an additional week; and central incisor deviations less than 0.25 mm can be considered “on-track”. Other rules may specify that teeth marked “Do No Move” should not deviate from their treatment position and any deviation greater than 0.25 mm should result in an appointment. Rules in a doctor guidance template may allow conditionals based on a treatment plan and/or other factors. In some implementations, rules in a doctor guidance template may be written with a temporal frame of reference and/or based on patient historical data (e.g., historical information about patient guidance provided to a consumer/patient in the past and/or historical measurement information).

An example of how the elements of the system 100 may operate to provide intelligent patient guidance is shown in FIG. 1D. At an operation 180a, the dental consumer/patient system 102 may capture one or more images of a consumer/patient. The one or more images may comprise photos taken by the camera of the dental consumer/patient system 102. The one or more photos may be captured by intelligent photo guidance techniques described further herein. The one or more images may include various perspectives and/or views of the dentition of the consumer/patient. The one or more photos captured at operation 18 a need not include scan data, height map information, and/or data a clinical scanner uses to stitch together a mesh representation of consumer/patient's dentition. The one or more photos may reflect a state of a treatment plan that is intended for and/or is underway on the consumer/patient. As an example, the one or more photos may capture an initial assessment of the consumer/patient's dentition and/or reflect the patient's progress at a specified stage of a treatment plan. The dental consumer/patient system 102 may store images captured locally, in a networked folder, etc. At an operation 180b, the dental consumer/patient system 102 may send captured photos of the consumer/patient to the virtual dental care system 106. This operation may include a file and/or other data transfer over the computer-readable medium 104.

At an operation 180c, the dental professional system 150 may gather treatment parameters for the consumer/patient. As noted herein, the treatment parameters may include doctor-preference parameters, per-patient parameters, etc. At an operation 180d, the dental professional system 150 may send the treatment parameters to the virtual dental care system 106. This operation may include a file and/or transfer over the computer-readable medium 104. As noted herein, the treatment parameters may comprise doctor-preference parameters and/or per-patient parameters.

At an operation 180e, the virtual dental care system 106 may create and/or update a doctor guidance template with treatment parameters. As noted herein, the doctor guidance template may supply a template with one or more rules that a doctor can use to track implementation of a treatment plan to a consumer/patient. The doctor guidance template may accommodate one or more rules to perform guidance deconfliction and/or prioritize various forms of action given doctor preferences, patient attributes, etc. The virtual dental care system 106 may store a doctor guidance template in any relevant format, including but not limited to any transitory and/or non-transitory medium. The virtual dental care system 106 may, in an operation 180f, send a doctor guidance template to the dental professional system 150.

At an operation 180g, the dental professional system 150 may process instructions to review, edit, and/or approve a doctor guidance template. In some implementations, the dental professional system 150 may provide a doctor with a user interface and/or other software that allows the doctor to review doctor guidance templates, make any changes to a doctor guidance template, and/or approve/finalize a doctor guidance template so that it can be applied to a specific patient, such as the consumer/patient using the dental consumer/patient system 102. As an example, in some implementations, a doctor may provide instructions to override a specific part of a doctor guidance template based on one or more factors, such as factors related to specific attributes of a specific consumer/patient. The dental professional system 150 may, in an operation 180h, send a reviewed/edited/approved doctor guidance template to the virtual dental care system 106. This operation may occur as a file and/or data transfer over the computer-readable medium 104.

At an operation 180i, the virtual dental care system 106 may use the captured photos and optionally the guidance template to generate intelligent patient guidance rules (e.g., rules that guide application of the treatment parameters to the consumer/patient). In some implementations, the virtual care dental system 106 may use the captured photos that were captured at the dental consumer/patient system 102 and a doctor guidance template reviewed, edited, and/or approved by the dental professional system 150 to generate intelligent patient guidance rules for the consumer/patient. At an operation 180j, the virtual care dental system 106 can generate patient guidance instructions using the intelligent patient guidance rules. Patient guidance instructions may take the form of instructions to the consumer/patient to take specific actions (add/change a dental appliance, wear a dental appliance longer or shorter than initially prescribed), may take the form of instructions to modify appointments and/or tasks, and/or may take the form of instructions to interact with the doctor in new and/or modified ways (e.g., draw attention to an area of dentition that is of increased interest).

At an operation 180k, the virtual dental care system 106 may provide patient guidance instructions to the dental consumer/patient system 102 and/or the dental professional system 150. This operation may occur as a file and/or data transfer over the computer-readable medium 104.

At an operation 180k, the dental consumer/patient system 102 may guide a consumer/patient using patient guidance instructions. In various implementations, the dental/consumer system 102 may present a consumer/patient with automated and/or interactive software elements that instruct the consumer/patient to take specified actions in relation to their treatment plans. As noted herein, example actions include instructions to change a dental appliance, instructions to keep a dental appliance beyond an initially prescribed time, use a supplemental dental appliance at a specific time/location, set an appointment for a specific condition and/or at a specific time/place, etc. At an operation 180l, the dental professional system 150 may guide the doctor with patient guidance instructions. In various implementations, the dental professional system 150 may present a doctor with automated and/or interactive software elements that, e.g., set appointments for a patient, notify a doctor about one or more conditions and/or regions of a consumer/patient's dentition to focus on, etc.

In some embodiments, the elements of the system 100 (e.g., the virtual dental care modules 108 and/or the virtual dental care datastore(s) 120) may be operative to provide photo-based refinements to users of the dental professional system 150. “Photo-based refinements,” as used herein, may include tools that allow a doctor performing virtual dental care to prescribe orders for consumers/patients whose treatments deviate from an intended course of treatment. The tools may use photos and may avoid requirements to rescan (e.g., perform a second and/or subsequent clinical scan after an initial clinical scan) the consumer/patient and/or provide a live evaluation of the consumer/patient, e.g., at the doctor's office. In some implementations, photo-based refinements may provide tools for a doctor to create a secondary (e.g., a refined) treatment plan remotely without ever physically seeing and/or evaluating a consumer/patient. Photo-based refinements may optimize one or more camera parameters to align a consumer/patient's treatment plan to photos captured by/for the consumer/patient. Photo-based refinements may also optimize one or more pose parameters (e.g., location parameters, orientation parameters, etc.) of a consumer/patient's teeth to ensure the teeth are in appropriate spaces. As noted herein, photo-based refinements may be displayed to doctors as user interface elements (e.g., overlays) representing a consumer/patient's dentition in relation to a treatment plan. Photo-based refinements can be used to plan one or more refinement treatment plans using 3D tooth shapes from a primary treatment plan and/or locations found using the techniques described herein; as noted herein, this information may be used to plan one or more new/refined treatment plans.

An example of how the elements of the system 100 may operate to provide photo-based refinements is shown in FIG. 1E. At an operation 190a, the dental consumer/patient system 102 may capture one or more images of a consumer/patient at a particular time, e.g., at one or more time during the course of virtual dental care. The one or more images may comprise photos taken by the camera of the dental consumer/patient system 102. The one or more photos may be captured by intelligent photo guidance techniques described further herein. The one or more images may include various perspectives and/or views of the dentition of the consumer/patient. As an example, the one or more images may include a plurality of images that represent more than one perspective of the consumer/patient's dentition. For instance, the images may be taken from anterior, left buccal, right buccal, and/or other perspectives. As noted herein, the one or more images may be captured as the consumer/patient is intelligently guided to take photos of their dentition. The one or more photos captured at operation 190a need not include scan data, height map information, and/or data a clinical scanner uses to stitch together a mesh representation of consumer/patient's dentition. The one or more photos may reflect a state of a treatment plan that is intended for and/or is underway on the consumer/patient. As an example, the one or more photos may capture an initial assessment of the consumer/patient's dentition and/or reflect the patient's progress at a specified stage of a treatment plan. The dental consumer/patient system 102 may store images captured locally, in a networked folder, etc. At an operation 190b, the dental consumer/patient system 102 may send captured photos of the consumer/patient to the virtual dental care system 106. This operation may include a file and/or other data transfer over the computer-readable medium 104.

At an operation 190c, the dental professional system 150 may request a first treatment plan for the consumer/patient. In some implementations, a doctor may, through instructions provided to the dental professional system 150, request a first treatment plan for a consumer/patient. The first treatment plan may comprise any set of instructions to address a dental condition of the consumer/patient. As an example, the first treatment plan may include instructions to move a consumer/patient's teeth from a first arrangement toward a target arrangement. The first treatment plan may prescribe use of successive dental appliances (e.g., a plurality of successive aligners shaped to receive and resiliently reposition a consumer/patient's teeth from the initial arrangement toward the target arrangement). The first treatment plan may include restoring attributes of a consumer/patient's dentition using crowns, bridges, implants, and/or other restorative dental appliances. In various implementations, the first treatment plan is based on a clinical scan, such as a clinical scan that occurred before the operation 190a.

At an operation 190d, the dental professional system 150 may send the request for the first treatment plan to the virtual dental care system 106. This operation may occur as a file and/or data transfer over the computer-readable medium 104.

At an operation 190e, the virtual dental care system 106 may retrieve the first treatment plan in response to the request for the first treatment plan. Retrieving the first treatment plan may involve providing instructions to a treatment datastore to retrieve a clinical data file associated with a consumer/patient. The clinical data file may represent an initial position of the consumer/patient's dentition, an intended target position of the consumer/patient's dentition, and/or a plurality of intermediate positions to move the consumer/patient's dentition from the initial position toward the intended target position. In some implementations, the clinical data file may include specific clinical preferences (stage(s) at which interproximal reduction (IPR) was performed, locations and/or times of application of dental auxiliaries applied during the first treatment plan, etc.). The clinical data file may also include clinical preferences of the doctor who managed prescription of the first treatment plan as well as specific attributes of dental appliances used to implement the first treatment plan.

At an operation 190f, the virtual dental care system 106 may identify an intended arrangement of a first treatment plan at the particular time that the photos of the consumer/patient were taken at the dental consumer/patient system 102. The virtual dental care system 106 may, e.g., use a length of time since initial implementation of the first treatment plan, spatial relationships between teeth in the photos captured at the dental consumer/patient system 102, and/or other information to identify the stage of the first treatment plan at which the photos were captured at the dental consumer/patient system 102. The virtual dental care system 106 may further evaluate a file that represents the intended arrangement of the identified stage of the first treatment plan to identify 3D structures, e.g., meshes corresponding to the identified stage of the first treatment plan.

At an operation 190g, the virtual dental care system 106 may evaluate photo parameters of the photos captured at the dental consumer/patient system 102 to generate alignment data, e.g., data representing an alignment of the intended arrangement of the first treatment plan to the photos. In some implementations, the virtual dental care system 106 optimizes 3D parameters from the images captured at the dental consumer/patient system 102. Examples of 3D parameters that may be optimized include camera parameters, location parameters, orientation parameters, etc. 3D parameter optimization may be performed using a variety of techniques, such as differential rendering, expectation maximization, etc. Applicant hereby incorporates by reference the following applications as if set forth fully here: U.S. Pat. App. Ser. No. 62/952,850, U.S. patent application Ser. No. 16/417,354; U.S. patent application Ser. No. 16/400,980; U.S. patent application Ser. No. 16/455,441; U.S. patent application Ser. No. 14/831,548 (now U.S. patent Ser. No. 10/248,883); and US20220023008A1, filed on Jul. 22, 2021. Once photo parameters are evaluated/optimized, the virtual dental care system 106 may use those photo parameters to determine places where the consumer/patient's teeth are not tracking to the first treatment plan. For instance, the virtual dental care system 106 may evaluate where the consumer/patient's teeth are in intended locations/orientations as well as where teeth deviate from intended locations/orientations.

At an operation 190h, the virtual care dental system 106 may generate an alignment mesh (e.g., an updated, segmented mesh) using the alignment data. The alignment mesh may comprise a 3D representation of the consumer/patient's dentition that reflects the photos taken at the consumer/patient system 102. At an operation 190i, the virtual care dental system 106 may evaluate the first treatment plan for modifications using the alignment mesh. The virtual dental care system 106 may identify locations where the consumer/patient's teeth are off-track and/or deviating from an intended arrangement prescribed by the first treatment plan. The virtual dental care system 106 may determine one or more modifications to the treatment plan (e.g., advancing to a subsequent stage of treatment early, or remaining in a current stage of treatment longer than originally planned), and may store any modifications in a clinical data file associated with the consumer/patient. At an operation 190j, the virtual dental care system 106 may send proposed modifications to a doctor and/or to a patient. This operation may occur as a file and/or data transfer over the computer-readable medium 104.

At an operation 190k, the dental professional system 150 may present and/or facilitate review of proposed modifications to the doctor. In various implementations, the dental professional system 150 shows a doctor the proposed modifications on a 3D model and/or images representing the consumer/patient's dentition. The dental professional system 150 may further allow the doctor to accept, reject, and/or further modify the 3D model and/or the images. As an example, the dental professional system 150 may allow the doctor to further move positions of dental auxiliaries, modify aligners and/or force systems, modify stages at which IPR is performed, etc. At an operation 190l, the dental professional system 150 may send reviewed modifications to the virtual dental care system 106, e.g., as a file and/or data transfer over the computer-readable medium 104. At an operation 190m, the virtual dental care system 106 may update the first treatment plan with the reviewed modifications. In various implementations, the virtual dental care system 106 updates a clinical data file associated with the consumer/patient with the reviewed modifications.

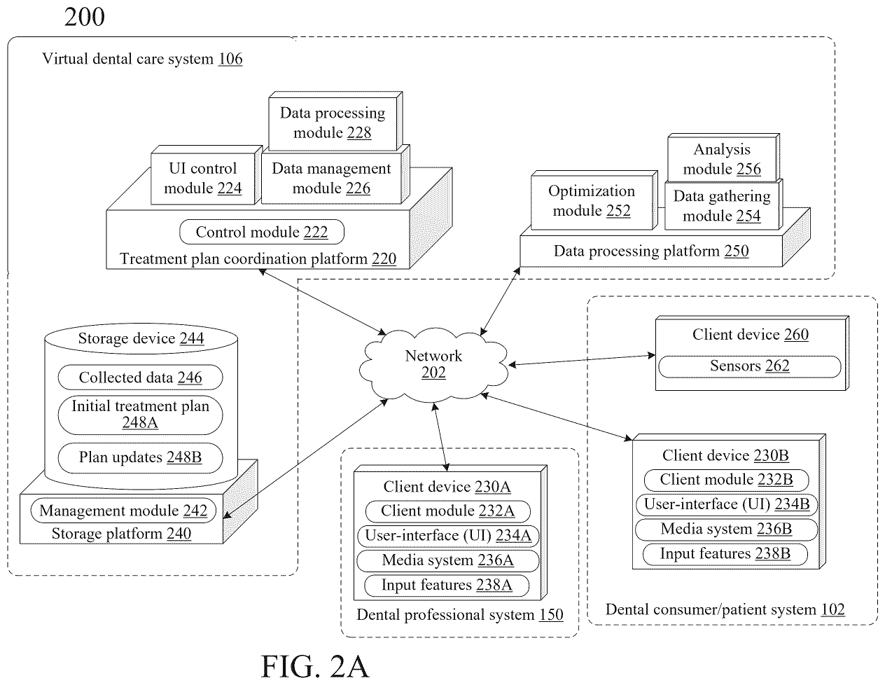

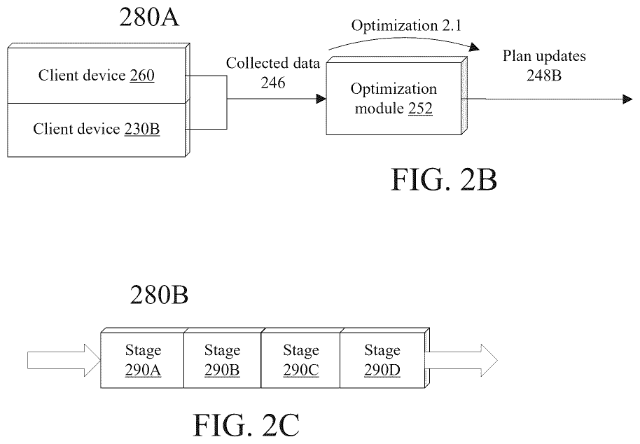

FIG. 2A illustrates an example system architecture 200 capable of supporting an optimization module for optimizing a treatment plan, in accordance with one embodiment of the present disclosure. The treatment architecture 200 may correspond to system 100 of FIG. 1A in some embodiments. For example, system architecture may include a dental consumer/patient system 102, a dental professional system 150 and a virtual dental care system 106. In embodiments, virtual dental care system 106 includes a treatment plan coordination platform 220, a data processing platform 250 and/or a storage platform 240. In embodiments, dental consumer/patient system 102 includes a client device 230B and/or one or more connected diagnostics monitoring client device(s) 260. In embodiments, dental professional system 150 includes a client device 230A. In some embodiments, any of the treatment plan coordination platform 220, the client device 230A, the client device 230B, the storage platform 240, the data processing platform 250, and the one or more connected diagnostics monitoring client device(s) 260, can include, can be, or can otherwise be connected to one or more computing devices (such as a rackmount server, a router computer, a server computer, a personal computer, a mainframe computer, a laptop computer, a tablet computer, a desktop computer, etc.), one or more storage devices (e.g., hard disks, memories, databases), networks, software components, and/or hardware components capable of connecting to system 200. In embodiments a platform may support any number of discrete software or hardware, or combination of such, portions, which may be referred to as modules.

In some embodiments, network 202 may connect the various platforms and/or devices, which can include a public network (e.g., the Internet), a private network (e.g., a local area network (LAN) or wide area network (WAN)), a wired network (e.g., Ethernet network), a wireless network (e.g., an 802.11 network or a Wi-Fi network), a cellular network (e.g., a Long Term Evolution (LTE) network), routers, hubs, switches, server computers, and/or a combination thereof.

In some embodiments, the treatment plan coordination platform 220 may facilitate or host services for coordinating HCP-patient communications relating to an on-going treatment plan for a patient. In embodiments, the treatment plan coordination platform 220 may host, leverage, and/or include several modules for supporting such system functionalities. For instance, in embodiments, platform 220 can support and/or integrate a control module (e.g., control module 222), for performing overall control of modules and devices associated with the platform, and a user-interface (UI) control module (e.g., UI control module 224), for performing generation, and other processes associated with a UI that will be presented through associated client devices. Platform 220 can support a data management module 226, that may gather and manage data from storage and modules (such as patient, or plan data gathered from storage device 244 and storage platform 240), and a data processing module (e.g., data processing module 228), that may process, transmit, and receive, incoming and outgoing data from client device 230A and/or client device 203B. Such modules may work collaboratively, and communicate internally, or externally (e.g., to further systems and/or through APIs), to facilitate virtual meeting capabilities for users across a range of client devices. Each module may include hardware, firmware, and/or software configured to provide a described functionality.

In some embodiments, platform control module 222 may orchestrate the overall functioning of the treatment coordination platform 220. In some cases, platform control module 222 can include algorithms and processes to direct the setup, data transfer, and processing required for providing and receiving data associated with a treatment plan from connected devices (e.g., the client device 230A, the client device 230B). For example, when a user initiates engagement with the treatment plan coordination system 200, the control module 222 may initiate and manage the associated process, including allocating resources, determining routing pathways for data and data streams, managing permissions, and so forth interact with client devices to establish and maintain reliable connections and data transfer. Control module 222 may also control internal modules of the treatment coordination platform 220 (e.g., modules that are within the treatment coordination platform 220).

UI control module 224 may perform user-display functionalities of the system such as generating, modifying, and monitoring the individual UI(s) and associated components that are presented to users of the platform 220 through a client device. For example, UI control module 224 can generate the UI(s) (e.g., UIs 234A-B of client devices 230A-B) that users interact with while engaging with the treatment coordination system.

A UI may include many interactive (or non-interactive) visual elements for display to a user. Different UIs may be provided to HCPs and to patients, each including one or more distinct visual elements. Such visual elements may occupy space within a UI and may be visual elements such as windows displaying video streams, windows displaying images, chat panels, file sharing options, participant lists, and/or control buttons for controlling functions such as client module navigation, file upload and transfer, controlling communications functions such as muting audio, disabling video, screen sharing, etc. The UI control module 224 may work to generate such a UI, including generating, monitoring, and updating the spatial arrangement and presentation of such visual elements, as well as working to maintain functions and manage user interactions, together with the control module 222. Additionally, the UI control module 224 may adapt a user-interface based on the capabilities of client devices. In such a way the UI control module 224 can provide a fluid and responsive interactive experience for users of the treatment coordination platform.

In some embodiments, the data management module 226 may be responsible for storage and management of data. This may include gathering and directing data from client devices. In embodiments, module 226 may communicate and store data, including to and/or from storage platforms and storage devices (e.g., such as storage device 244), etc. For instance, once an initial treatment plan (e.g., initial plan 248A) has been established, data management module 226 may perform tasks such as gathering and directing such data to the UI control module 224, or to the storage platform 240, or to client devices 230A-B (with the aid of processing module 228). In embodiments, data management module 226 can also be primarily responsible for communicating other types of data. Such data may include video data, image data, sensor data, modeling data, transcripts, communications data, metadata, etc.

In embodiments, data management module 226 may include a database interface module (DIM). In embodiments, module 226 may not only direct storage of data associated with the system, but may also generate and manage metadata, including titles, transcriptions, timestamps, shared documents, descriptions, texts tags, thumbnails, records, reminders, and so one and so forth.

In some embodiments, data management module 226 may work hand-in-hand with data processing module 228, which may receive, process, and transmit data with low-latency. In some cases, data processing module 228 may be equipped to receive, transmit, encode, decode, compress, or otherwise process data for efficient delivery to or from devices, modules, or platforms, etc. In embodiments, data processing module 228 may be controlled or directed by data management module 226, and/or control module 222.

In embodiments, processing module 128 and/or management module 126 may also include advanced capabilities through access to more sophisticated computer algorithms, and may implement advanced processing features such as noise reduction, data enhancement (e.g., video quality upscaling), data encryption, ensuring transmission integrity etc., all with the intent of optimizing and protecting data transfer and processing in support of the control module, treatment plan coordination platform, and its users at large.

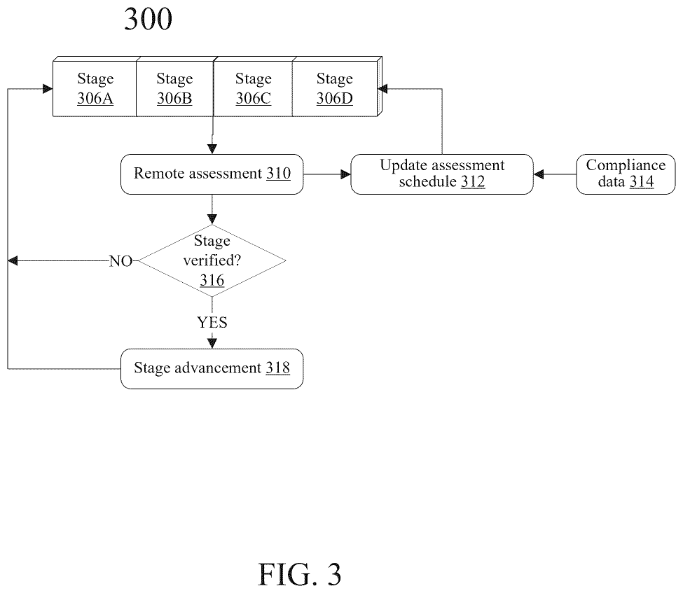

In embodiments, data that is transmitted, managed, and/or manipulated by data management module 126 and data processing module 228 may include any kind of data associated with a treatment plan, including (e.g., treatment plan schedules, dates, times, etc.), patient data (e.g., such as images, values, sensor data, etc.). Further types of such data will be discussed below and additionally with respect to FIGS. 2B-3.

Some data, such as textual input (e.g., comments or textual commands associated with the client module navigation, etc.) and control commands (user interactions with control elements of the UI), etc., may not be received by the data processing module or data management module, but instead by the control module 222, which may process and forward such received data to connected devices and modules as needed. For example, in instances where a client device engages or activates a system function (e.g., such as requesting a video-call, or initiating a data retrieval function), user inputs from one or more client devices may be received directly by platform control module 222.

In such a manner, a user of a client device may transfer commands, inputs, and other data for any of the modules of platform 220 to execute. In such a way, transmitting, receiving, and processing of data by the treatment plan coordination platform 220 from one or more connected client devices (e.g., client devices 230A-B, device 260, etc.) may be coordinated by the control module 222 in tandem with other associated modules and platforms, as seen in FIG. 2A.

In embodiments, the system 200 may leverage a data processing platform 250, for performing processes associated with data collected by the client devices. In embodiments, treatment plan coordination platform 220 may include an optimization module 252, a data gathering module 254, and an analysis module 256. Optimization module 252 may intake collected data (e.g., collected data 246) from a patient's client device and process such data to generate optimizations, or plan updates 248B to an initial plan 248A. Data gathering module 254 may collect and organize patient data and corresponding plan updates as produced by optimization module 252 (in some embodiments, such functions may be performed via data management module 226). Analysis module 256 may analyze the collected data to identify significant trends, characterizations corresponding to specific treatment plans, associated data segments, and insights within the data. Such functions and embodiments of platform 250 and the associated modules will be further described in detail below and with respect to FIGS. 2B-4C.

In some embodiments, one or more client devices (e.g., client device 230A-B) can be connected to the system 200. In embodiments, the client device(s) can each include computing devices such as personal computers (PCs), laptops, mobile phones, smart phones, tablet computers, netbook computers, notebook computers, network-connected televisions, etc. In some embodiments, client device(s) may also be referred to as “user devices.”

In embodiments, client devices (e.g., client device 230A, client device 230B) connected to the system can each include a client module (e.g., client module 232A, client device 232B). In some embodiments, the client module may be an application that provides the user interface (UI) (e.g., client module 234A, client device 234B) and manages transmissions, inputs, and data to and from platform 220. In some embodiments, the client module that provides the UI can be, or can include, a web browser, a mobile application, a desktop application, etc.

Client devices, under direction by the treatment coordination platform when connected may present or display a UI (e.g., UI 234A, UI 234B) to a user of the respective client device. In embodiments, the UI may be generated and controlled by control module 224. In embodiments, the UI may be generated locally at the client device, e.g., through client modules 232A-B. A UI may include various visual elements and regions, and may be the primary mechanism by which the user interfaces with the client module, the treatment plan coordination platform, and the system at large. In some embodiments, the UI(s) of the client device(s) can include multiple visual elements and regions that enable presentation of information, for decision-making, content delivery, etc. to a user of the device. In some embodiments, the UI may be referred to as a graphical user interface (GUI).

In some embodiments, the system (or any associated platforms), may transmit any data, including audio, video, image, and textual data, to the client device to be interpreted by client module 232A-B, and displayed via the UI of the respective client device. Such data that can be transmitted to the client device through modules 232A-B can include, for example, UI information, textual information, video, or audio streaming data associated with the HCP-patient communications, control, or navigation data, etc. In some embodiments, a client module 232A-B (e.g., a dedicated application) incorporated within the client device 230A-B and may perform function associated with the end-user interface.

In embodiments, connected client devices may also collect input from users through input features 238A-B. Input features 238A-B may include UI features, software features, and/or requisite hardware features (e.g., mouse and keyboard, touch screens, etc.) for inputting user requests, and/or data to the treatment plan coordination system. Input features 238A-B of client devices 230A-B can include space, regions, or elements of the UI 234A-B that accept user inputs. For example, input features 238A-B may be visual elements such as buttons, text-entry spaces, selection lists, drop-down lists, control panels, etc.

In embodiments, input features 238A-B may include portions of an associated media system e.g., a camera, microphone, and/or similar elements of media system 236A-B, to transmit or intake further user-inputs. In embodiments, the media system of the client device may include at least a display, a microphone, speakers, and a camera, together with other media elements as well. Such elements (e.g., speakers, or a display) may further be used to output data, as well as intake data or inputs.

In embodiments, a client module (e.g., client module 232A, client module 232B) may execute a series of protocols to access and control media system hardware resources, in some cases accessing device-level APIs or drivers that interact with the underlying hardware of a media system. Through such, or similar, protocols, client modules may utilize any of the components of a client device media system for specific functionalities within the context of virtual dental care. For instance, in embodiments, a display of the media system may be employed by the client module (under direction from the treatment coordination platform 220) to render the UI. In embodiments, graphical elements may be presented or displayed to the user via the display and the UI. The client module of a device may direct rendering commands to the display to update the screen with relevant visual information. Similarly, and/or simultaneously, in embodiments, a camera or imaging sensor of the media system may capture image and/or video input from the user to transmit. In embodiments, the client module may process, encode, and transmit such data from the client device, over the network, to the treatment plan coordination platform 220.

As will be discussed further below, in embodiments, a client module 232B associated with a patient client device 230B may transfer patient data (including captured audio and/or image data), biomarker data, patient observations (e.g., of experienced pain, looseness of aligners, aligner fit, etc.), force or pressure data measured by dental appliances, etc. associated with the treatment plan to treatment plan coordination platform 220, which may forward, process and/or store such data. In embodiments, such data may be forwarded from a first client device to a second client device.

As will be further discussed below, in embodiments, data collected from a patient client device 230B can be stored in storage device 244 as collected data 246. Such collected data 246 may include collected data associated with a single patient, and a single patient dental treatment plan. Alternatively, data associated with multiple patients and/or multiple dental treatment plans and separate procedures may be stored as individual data segments of collected data 246.

In embodiments, a first client device 230B may gather data and inputs from a patient, to be transmitted and displayed to an HCP at a second client device 230A. For instance, in embodiments, device 230B may belong to a patient, while client device 230A may belong to an HCP. In embodiments, such a pairing and configuration can facilitate communication and data transfer between both parties. For example collected patient data from client device 230B can be transmitted and displayed to an HCP at client device 230A, which may then transmit instructions, guidance, or any other kinds of data back to the patient client device 230B. In embodiments, such data can include updates to a treatment plan.

In embodiments, client devices 230B connected to the system 200 can collect data from more than one client device 260 associated with a single user. For example, in embodiments, both client device 230A and 260 may be associated with one user. In embodiments, client devices 230A and 260 may be different types of client devices for collecting different types of data. For example, in embodiments, client device 230A may be a personal computing device (e.g., a mobile phone or tablet of a patient), while client device 260 may be a dedicated, or embedded, diagnostics monitoring device. For example, in some embodiments, client device 230A may be a personal phone or similar device, while client device 260 may be a scanner for obtaining three-dimensional (3D) data of a dental site in a patient's oral cavity (or another imaging device including a camera) and may be operatively connected to a personal client device (e.g., device 230A). In some embodiments, more than two, including any number of, client devices may be used to gather and monitor oral health data from the patient.

Client device 260 may be, for example, a dental device worn in a mouth of a patient, where the dental device has one or more strain gauges or other force sensors for detecting an amount of force exerted on the dental device by one or more teeth of a patient, or conversely, an amount of force exerted on the one or more teeth of the patient by the dental device. In some examples, the dental device may include a sensor for determining an amount of tooth movement (e.g., optical, or electrical sensor pairs places on opposing sides of an intraoral cavity that can determine distances between them to determine displacement, capacitance sensors that can measure distances). In another example, client device 260 may be a device worn in a mouth of a patient that measures properties such as pH, bio indicators or biomarkers, saliva quantity, temperature, and so on. Any and all such sensor data may be collected by client device by sensors 262 and provided to client device 230B for forwarding on to treatment plan coordination platform 230 and/or may be provided directly to treatment plan coordination platform 220 via network 202. More information about sensors that can be included in dental devices for collecting data is described in U.S. Pat. No. 10,470,847, issued Nov. 12, 2019 (including various types of sensors for compliance detection), U.S. Pat. No. 11,779,243, issued Oct. 10, 2023 (including capacitance sensors in microfluidic arrays to detect stress relaxation), U.S. Pat. No. 11,576,766, issued Feb. 14, 2023 (including biosensors derive tooth movement based on detected biomarkers, e.g., cytokines), U.S. Pat. No. 11,432,908, issued Sep. 6, 2022 (including sensors to detect force/impedance), and U.S. Pat. No. 7,854,609, issued Dec. 21, 2010 (including reservoirs with an agent that is released when aligner is worn, and the agent concentration is measured to determine wear time), each of which is incorporated by reference herein in its entirety.

In embodiments, client device 260 may gather and transmit patient data without a traditional display or UI. For example, in embodiments, diagnostics monitoring client device 260 may be embedded into a bodily location of a patient. E.g., in embodiments, diagnostics monitoring client device 260 may be, or be a part of a dental appliance, fixture, or apparatus. E.g., in embodiments, client device 260 may be, or be a part of, a dental appliance such as aligners, braces, palatal expanders, or any similar dental device. In embodiments, client device 260 may be any client device that collects data from a patient, such as a client device that is a dedicated imaging system, or a client device that is associated with the case, or holder for the aligner (e.g., in such a case, the case may intake measurements from the aligner when it is stored in the case, such as deformation measurements, oral pH measurements, etc.).

In embodiments, diagnostics monitoring client device 260 may be equipped with various sensors 262, which may be specific types of input features, including any kind of sensors and input mechanisms for gathering patient data. For example, in embodiments, the device may include biological sensors for capturing real-time measurements (e.g., collected data) associated with a patient. In embodiments, such sensors can include pressure sensors (including touch or tactile sensors), motion sensors (e.g., accelerometers, vibration sensors, etc.), audio sensors (e.g., microelectromechanical system (MEMS) microphones, biomarker sensors, chemical sensors (e.g., such as a pH sensor), optical sensors (e.g., such as a color sensors or light sensors), image sensors, temperature sensors, heart-rate sensors, electrical sensors (e.g., capacitive, resistive, conductive, etc.), electrodes, proximity sensors, or any combination of such or similar sensors to gather sensor data associated with tooth position, movement, or any other data relevant to a dental treatment plan.

Examples of proximity sensors suitable for use with the embodiments herein include capacitive sensors, resistive sensors, inductive sensors, eddy-current sensors, magnetic sensors, optical sensors, photoelectric sensors, ultrasonic sensors, Hall Effect sensors, infrared touch sensors, or surface acoustic wave (SAW) touch sensors. In embodiments, a proximity sensor can be activated when within a certain distance of the sensing target. The distance can be about less than 1 mm, or within a range from about 1 mm to about 50 mm. In some embodiments, a proximity sensor can be activated without direct contact between the sensor and the sensing target (e.g., the maximum sensing distance is greater than zero).

In some embodiments, a proximity sensor is activated when in direct contact with the sensing target (the sensing distance is zero), also known as a touch or tactile sensor. Examples of touch sensors include capacitive touch sensors, resistive touch sensors, inductive sensors, pressure sensors, and force sensors. In some embodiments, a touch sensor is activated only by direct contact between the sensor and the sensing target (e.g., the maximum sensing distance is zero). Some of the proximity sensor types described herein (e.g., capacitive sensors) may also be touch sensors, such that they are activated both by proximity to the sensing target as well as direct contact with the target.

One or more proximity sensors may be integrated in the intraoral dental appliance and used to detect whether the appliance is in proximity to one or more sensing targets. The sensing targets can be an intraoral tissue (e.g., the teeth, gingiva, palate, lips, tongue, cheeks, or a combination thereof). For example, proximity sensors can be positioned on the buccal and/or lingual surfaces of an appliance in order to detect features such as tooth position, movement, or dental appliance usage (e.g., compliance) based on proximity to and/or direct contact with the patient's cheeks and/or tongue. As another example, one or more proximity sensors can be positioned in the appliance so as to detect features such as tooth position, movement, or dental appliance usage (e.g., compliance) based on proximity to and/or direct contact with the enamel and/or gingiva. In some embodiments, multiple proximity sensors are positioned at different locations appliance so as to detect proximity to and/or direct contact with different portions of the intraoral cavity.

Alternatively or in combination, one or more sensing targets can be coupled to an intraoral tissue (e.g., integrated in a dental auxiliary on a tooth), or can be some other component located in the intraoral cavity (e.g., a metallic filling). Alternatively or in combination, one or more proximity sensors can be located in the intraoral cavity (e.g., integrated in a dental auxiliary on a tooth) and the corresponding sensing target(s) can be integrated in the intraoral appliance. Optionally, a proximity sensor integrated in a first appliance on a patient's upper or lower jaw can be used to detect a sensing target integrated in a second appliance on the opposing jaw or coupled to a portion of the opposing jaw (e.g., attached to a tooth), and thus detect proximity and/or direct contact between the patient's jaws.

The proximity sensor may be a capacitive sensor activated by charges on the sensing target. The capacitive sensor can be activated by charges associated with intraoral tissues or components such as the enamel, gingiva, oral mucosa, saliva, cheeks, lips, and/or tongue. For example, the capacitive sensor can be activated by charges (e.g., positive charges) associated with plaque and/or bacteria on the patient's teeth or other intraoral tissues. In such embodiments, the capacitive sensing data can be used to determine whether the appliance is being worn, and optionally the amount of plaque and/or bacteria on the teeth. As another example, the capacitive sensor can be activated by charges associated with the crowns of teeth, e.g., negative charges due to the presence of ionized carboxyl groups covalently bonded to sialic acid.

Alternatively or in combination, the intraoral tissue can serve as the ground electrode of the capacitive sensor. Optionally, a shielding mechanism can be used to guide the electric field of the capacitive sensor in a certain location and/or direction for detecting contact with a particular tissue.

Alternatively or in combination, a monitoring client device can include one or more vibration sensors configured to generate sensor data indicative of intraoral vibration patterns. Examples of vibration sensors include audio sensors (e.g., MEMS microphones), accelerometers, and piezoelectric sensors. The intraoral vibration patterns can be associated with one or more of: vibrations transferred to the patient's teeth via the patient's jawbone, teeth grinding, speech, mastication, breathing, or snoring. In some embodiments, the intraoral vibration patterns originate from sounds received by the patient's ear drums. The intraoral vibration patterns may also originate from intraoral activities, such as teeth grinding, speech, mastication, breathing, snoring, etc. The sensor data generated by the vibration sensors can be processed to detect features such as tooth position, movement, or dental appliance usage (e.g., compliance). For instance, the monitoring device can include a processor that compares the detected intraoral vibration patterns to patient-specific intraoral vibration patterns to determine whether the appliance is being worn on a patient's teeth. In some embodiments, the processor is trained using previous data of patient-specific intraoral vibration patterns, and then determines whether the appliance is being worn by matching the measured patterns to the previous patterns. Alternatively or in combination, appliance usage can be determined by comparing the measured vibration patterns to vibration patterns obtained when the appliance is not being worn.

Alternatively or in combination, a monitoring client device can include one or more optical sensors or image sensors configured to detect features such as tooth position, movement, or dental appliance usage (e.g., compliance) based on optical signals. For example, the optical sensors can be color sensors (e.g., mono-channel color sensors, multi-channel color sensors such as RGB sensors) configured to detect the colors of intraoral tissues. In some embodiments, one or more color sensors can be integrated into the intraoral appliance so as to be positioned adjacent to certain intraoral tissue (e.g., enamel, gingiva, cheeks, tongue, etc.) when the appliance is worn in the mouth. The device can capture data associated with dentition as well as whether the appliance is currently being worn based on the types and visibility of colors detected by the sensors. In such embodiments, the monitoring device can include one or more light sources (e.g., LEDs) providing illumination for the color sensors.

As another example, the monitoring device can include one or more emitters (e.g., a LED) configured to generate optical signals and one or more optical sensors (e.g., a photodetector) configured to measure the optical signals. For example, an emitter can be positioned such that when the appliance is worn, the optical signal is reflected off of a surface (e.g., an intraoral tissue, a portion of an intraoral appliance) in order to reach the corresponding optical sensor. In some embodiments, when the appliance is not being worn, the optical signal is not reflected and does not reach the optical sensor. Accordingly, data indicative of a patient's detention, as well as compliance data, can be determined via aspects of data captured via the optical sensor.

Alternatively or in combination, the monitoring devices of the present disclosure can include one or more magnetic sensors configured to detect appliance usage based on changes to a magnetic field. Examples of magnetic sensors suitable for use with the embodiments herein include magnetometers, Hall Effect sensors, magnetic reed switches, and magneto-resistive sensors. In some embodiments, the characteristics of the magnetic field (e.g., magnitude, direction) vary based on whether the appliance is currently being worn, proximity of teeth, etc., e.g., due to interference from intraoral tissues such as the teeth. Accordingly, the device can determine appliance usage and dentition data by processing and analyzing the magnetic field detected by the magnetic sensors.

Alternatively or in combination, a monitoring client device can utilize two or more magnets that interact with each other (e.g., by exerting magnetic forces on each other), and a sensor that detects the interaction between the magnets. For example, the sensor can be a mechanical switch coupled to a magnet and actuated by magnetic forces exerted on the magnet. As another example, the sensor can be configured to detect the characteristics (e.g., magnitude, direction) of the magnetic force exerted on a magnet by the other magnets. The magnets and sensor can each be independently integrated within a dental appliance or coupled to a tooth or other intraoral tissue to gather data associated with dentition and compliance.

In embodiments, a monitoring client device may include sensors for capture of data associated with bioagents in the intraoral cavity while an intra-oral appliance (e.g., aligner, palatal expander, etc.) is in use. Such apparatuses and methods may collect information (data), including data about tooth movement phases, via analysis of biomarkers in saliva or gingival crevicular fluid (GCF). For example, the data can be indicative of patient wearing compliance, the amount of tooth movement achieved, the amount of force and/or pressure actually applied to the teeth by the appliance, bone remodeling processes and stages, tissue health, bacterial activity in the oral cavity, or any combination thereof.

In embodiments, any biomarker may be targeted, particularly but not exclusively a biomarker present in saliva. Alternatively, the biomarker may be a biomarker in the blood, gingiva, or teeth. In some cases, the biomarker may be a biomechanical property, such as heart rate, blood pressure, etc. Biomarkers that may be present in saliva and may be targeted for sensing may include one or more of: Calgranulin-B, Serum albumin precursor, Immunoglobulin J chain, Ig alpha-1 chain C region, Cysteine-rich secretory protein 3 precursor (CRISP-3), Hemoglobin subunit beta, Stratifin, and soluble RANK Ligand (sRANKL). In embodiments, \the biomarker may be present in gingival crevicular fluid (GCF). For example, the biomarker may be one or more of: prostaglandin E2, Substance P, epidermal growth factor, transforming growth factor, receptor activator of nuclear factor kappa-B ligand (RANKL), Granulocyet macrophage colony stimulation factor, α2 microglobulin, Interleukin 1β, Myeloperoxidase, hyaluronic acid, and Chondroitin sulfate.

In embodiments, any appropriate biomarker or biomarkers may be designed to be sensed via a sensor included in sensors 162. For example, biomarkers may be biomolecules or byproducts of biomolecules that are present in the oral cavity (including saliva, GCF, and/or breath) and/or contaminants that may be present in the oral cavity, such as bacteria, yeast, etc. Biomolecules of particular interest include those that change in response to movement of the teeth due to an orthodontic procedure. Such biomarkers of interest may include protein biomarkers include Protein S100-A9 (e.g., S100 calcium-binding protein A9, Calgranulin-B), Serum albumin precursor, Immunoglobulin J chain, Ig alpha-1 chain C region, Cysteine-rich secretory protein 3 precursor (CRISP-3), Hemoglobin subunit beta (Hemoglobin beta chain, Beta-globin), and 14-3-3 protein σ (Stratifin, Epithelial cell marker protein 1) or any combination thereof or of similar biomarkers may be sensed via sensors 162.

In embodiments, any of the bio sensors described herein may include a bio-recognition component, a biotransducer component, and electronic system which may include a signal amplifier, processor, data logging units and data communication unit. In some instances, transducers and electronics can be combined such as in CMOS-based microsensor systems. The recognition component may be called a bioreceptor, may use a biomolecule from organisms or receptors modeled after biological systems to interact with the analyte of interest. This interaction may be measured by the biotransducer which outputs a measurable signal proportional to the presence of the target analyte in the sample. The processor can log the raw or processed data in the memory unit or transmit it to a receiver. The system can work actively if energized with battery, super-capacitor, or an energy harvesting unit, or it may perform passively upon being energized via induction using an external device, such as cell phone.

In embodiments, one or more sensors of sensors 162 may be positioned to collect data from a specific portion of a patient's mouth. For instance, in embodiments, a sensor can be located at any portion of an intraoral dental appliance, such as at or near a distal portion, a mesial portion, a buccal portion, a lingual portion, a gingival portion, an occlusal portion, or a combination thereof. In embodiments, a sensor can be positioned near a tissue of interest when the appliance is worn in the patient's mouth, such as near or adjacent the teeth, gingiva, palate, lips, tongue, cheeks, airway, or a combination thereof. For example, when the appliance is worn, the sensor can cover a single tooth, or a portion of a single tooth. Alternatively, the sensor can cover multiple teeth or portions thereof. In embodiments where multiple sensors are used, some or all of the monitoring devices can be located at different portions of the appliance and/or intraoral cavity. Alternatively, some or all of the sensors may be located at the same portion of the appliance and/or intraoral cavity.

In some embodiments, client device 160 may be a non-embedded, dedicated diagnostics monitoring device. For instance, in some embodiments, client device 160 may be a handheld probe, an optical scanner, or an imaging device, etc. For example, in embodiments client device 160 may be or include a scanner such as a probe (e.g., a handheld probe), that may optically capture three dimensional structures (e.g., by confocal focusing of an array of light beams). An example of such a scanner and/or probe is the iTero® intraoral digital scanner manufactured by Align Technology, Inc. Alternate examples of intraoral scanners include the 3M True Definition Scanner and the Cerec Omnicam manufactured by Sirona®.

In embodiments, such a scanner or scanning device may be used to perform intraoral scanning. In doing so, a user (e.g., the HCP or the patient) may apply the client device to one or more patient intraoral locations.

In embodiments, the scanning via such a client device may be divided into one or more segments including a lower buccal region of the patient, a lower lingual region of the patient, a upper buccal region of the patient, an upper lingual region of the patient, one or more preparation teeth of the patient (e.g., teeth of the patient to which a dental device such as a crown or an orthodontic alignment device will be applied), one or more teeth which are contacts of preparation teeth (e.g., teeth not themselves subject to a dental device but which are located next to one or more such teeth or which interface with one or more such teeth upon mouth closure), and/or patient bite (e.g., scanning performed with closure of the patient's mouth with scan being directed towards an interface area of the patient's upper and lower teeth).

In alternate, or additional embodiments, intraoral scan and/or image data may also include data from one or more varying image capture devices. For example, in embodiments, additional image capture may be performed via an x-ray device capable of generating standard x-rays (e.g., bite wing x-rays), panoramic x-rays, cephalometric x-rays, etc. Image capture may (additionally or alternatively) include an x-ray device capable of generating a cone beam computed tomography (CBCT) scan. Image capture may (additionally or alternatively) include a standard optical image capture device (e.g., a camera, such as a camera of a mobile phone) that generates two-dimensional or three-dimensional images or videos of a patient's oral cavity and dental arch. For example, in embodiments, an image capture device may be a mobile phone, a laptop computer, an image capture accessory attached to a laptop or desktop computer (e.g., a device that uses Intel® RealSense™ 3D image capture technology), and so on. Such a device may be operated by a patient, an acquaintance (e.g., a friend or family of the patient) or a professional in a workplace setting (e.g., a clinic). As described, such device(s) may generate 2D or 3D images that are sent to the dental treatment coordination platform and/or an HCP's client device.

Accordingly, collected data 246 may include 2D optical images, 3D optical images, virtual 2D models, virtual 3D models, 2D x-ray images, 3D x-ray images, etc. 3D optical images and/or virtual 3D models may be or include 3D point clouds in some embodiments.

In embodiments, sensors 262 may be of any kind to collect any kind of data associated with a patient's oral health, and/or data that is relevant for a dental treatment plan. Further specific data types that may be collected from such sensors will be described in further detail below, with respect to the description of collected data 246.

Once the diagnostics monitoring client device 260 has gathered the patient data, such data may be transmitted to the treatment plan coordination platform 220. In embodiments, client device 260 may include an onboard data processing module for standardizing, encrypting, and transmitting such data to platform 220. In embodiments, client device 260 may transmit such data first to a separate client device of the user (e.g., client device 230A-B), which may then transmit the data to treatment plan coordination platform 220. For example, in any number of connected client devices (e.g., client device 260) collected data may be stored in physical memory on the device and may be retrieved by another device (e.g., client device 230A) in communication with the monitoring apparatus. Retrieval may be done wirelessly, e.g., using near-field communication (NFC) and/or Bluetooth (BLE) technologies to use a smartphone or other hand-held device to retrieve the data.

Thus, any client device used for diagnostics monitoring, including a dedicated client device 260, may include the requisite hardware for enabling such transmissions of collected data. Such hardware may include requisite sensors as have been described, a CPU, an NFC communication module, an NFC antenna, a PCB, a battery, etc. In embodiments, client devices may further be or include cases or holders that may boost and/or relay the signals from the sensing portion of a device to a handheld device such as a smartphone; such cases or holders may be referred to as NFC-BLE enabled cases.

Thus, in some embodiments, such data be gathered remotely e.g., via a patient at their home (alternatively, such data may be collected at a clinic e.g., at an HCP's office). As previously mentioned, through such multiple, connected, client devices of the treatment plan coordination system, real-time or near-real-time collection and transmission of patient data can be accomplished, thus enhancing data collection while minimizing intrusiveness into a patient's lifestyle.

In embodiments herein, such patient data collected by a client device of a user, as described with respect to client device 260 and/or devices 230A-B, may be holistically referenced as collected data 246, which may be ultimately stored within storage device 244.

In some embodiments, the system may include storage platform 240, which may host and manage storage device 244. In embodiments, a management module 242 may be used to manage communications, and storage device 244. In some embodiments, platform 240 may be a dedicated server for supporting storage device 244 accessible via network 202.

A management module (e.g., management module 242) may reside at, or within, platform 240. The management module may oversee, regulate, and optimize operation associated with the storage device 244. The management module 242 may further accomplish tasks including, handling requests directed towards the storage device 244, ensuring the integrity and security of the data, managing backups, and orchestrating efficient use of storage resources provided by platform 240.

In some embodiments, when a data request or command is made to storage device 244, the request of command may first interface with the management module 242. The management module 242 may processes a request and determine the most efficient manner to execute the request using the resources of the storage platform 240. Subsequently, data operations may be performed on storage device 244 and data 246, leveraging the underlying capabilities of the storage platform 240. In some cases, storage device 244 may be a persistent storage that is capable of storing data and/or metadata, as well as data structures to tag, organize, and index the data and/or metadata. Storage device 244 may store at least collected data 246, initial plan 248A, and plan updates 248B.

In embodiments, collected data 246 may include any data that has been collected from client devices associated with the system. In embodiments, the collected data 246 may be data collected from one or more patient's before, after, or during a dental treatment plan. In embodiments, such data may be accessible and displayable via any of the connected client devices.

In embodiments, collected data 246 may include the oral health data acquired through multiple sources, such as an implanted and/or embedded client device and a connected device (as described above). In embodiments, these may be at least client device 230A and client device 260. In embodiments, such collected data 246 may include oral temperature data, oral pH data, pressure data (e.g., bite force or pressure otherwise sensed by a specific tooth, etc.), image data (including x-ray, or ultrasound, etc.), biomarker data, heart rate data, respiratory data, body temperature, image data, video data, textual data, and or raw data indicative of electrical parameters associated with health data (e.g., capacitance, resistance, conductance values, etc.). In embodiments, collected data 246 may be any kind of data associated with a patient's oral health, and/or data that is relevant for a treatment plan.

In embodiments, such collected data may include spatial positioning data, including 2D or 3D patient data. In embodiments, collected data may include image data which may be used to generate a virtual model (e.g., a virtual 2D model or virtual 3D model) of the real-time conditions of the patient's oral features and/or dentition (e.g., conditions of a tooth, or a dental arch, etc., may be modeled). To generate the virtual model, the system may register (i.e., “stitch” together) intraoral images generated from an intraoral scan (e.g., by an HCP using a clinical intraoral scanner, by the patient using an at-home intraoral scanner) and an imaging client device (e.g., a smart phone). In some embodiments, captured intraoral images may be integrated into a common reference frame by applying appropriate transformations to points of each registered image. In embodiments such images may be stored in 2D format, or as a model in 3D format.

In embodiments, and as will be further described with respect to FIG. 3, the collected data 246 may include compliance data. Compliance data may be an indicator of the level of adherence the patient has accomplished with respect to the established dental treatment plan (as established by the HCP). Such compliance data will be further described with respect to FIG. 3.

In embodiments, further “soft” data may be gathered through an interactive client device, such as client device 230B. For example, in embodiments, a client device with a UI may prompt the user to input particular information manually, such as oral hygiene habits, eating habits, discomfort or irregularities experienced, etc. In a non-limiting example of such interactivity, the system and/or client device may prompt a patient and/or user with a query. In a non-limiting example, the system and/or client device may prompt a patient and/or user with the query, “how tight are your aligners (1-5)?”, “do the aligners seem to be fitting well?”, “do you have all of your dental auxiliaries?”, “indicate a pain level of your teeth on a scale of 1-10”, or any other similar, or similar combination of, query(ies) to collect useful data. In embodiments, such a prompt may be, and/or illicit as a response, any form of freeform text. Thus, the collected data may include data that is collected in response to questions or prompts. Thus, the collected data may include data that is collected via a UI, such as data that is self-reported from a user. Such data may include data such a user's oral hygiene habits, eating habits, discomfort or irregularities experienced, etc., which may all be self-reported. Additionally, in embodiments, client devices equipped with cameras can also capture visual data, including images or videos of the oral cavity, which may then be processed and stored within collected data 246. Thus, collected data 246 may include image, video, text, sensor-gathered, or any other kind of data associated with a treatment plan, and the system at large.

In embodiments, data 246 may be encrypted and securely stored. In embodiments, data 246 (e.g., audio and video data) may be captured in various formats and encoded using different codecs, as dictated by the system requirements and user settings. For example, video data within storage device 244 may include video data (which may be encoded in formats like MP4, AVI, MKV, etc.), audio data (which may be encoded in formats such as AAC, MP3, or WAV., etc.), metadata (which can include a number of data such as video titles, descriptions, video durations, timestamps, upload information keywords, target frame rates, etc.), etc. One of ordinary skill in the art, having the benefit of this disclosure, will appreciate that there exist many useful data types and formats that may further be stored with data 246, and that the above list is non-exhaustive.

In embodiments, storage device 244 may further include an initial treatment plan 248A and plan updates 248B, as produced by optimization module 252.

In embodiments, the initial treatment plan 248A may function as, or be an initial, pre-defined treatment plan that consists of scheduled stages designed to sequentially correct and improve aspects of a patient's health. In some embodiments, the initial treatment plan may be a plan for improving aspects of a patient's oral health. In some cases, the plan can be an initial plan determined by an HCP, and based on portions of collected data 246, such as tests, documentation, medical history, etc. For example, in embodiments the initial treatment plan may be a multi-stage dental treatment plan initially been generated by an HCP (e.g., an orthodontist) after performing a scan of an initial pre-treatment condition of the patient's dental arch. In some embodiments, the initial treatment plan may begin at home (e.g., be based on a patient scan of his- or her-self) or at a scanning center. In embodiments, the initial treatment plan might be created automatically and/or by a professional (including an orthodontist) in a remote service center.

In embodiments, the initial dental treatment plan can be an orthodontic treatment plan, palatal expansion treatment plan, combined palatal expansion and orthodontic treatment plan, etc. generated based on intraoral scan data providing surface topography data for the patient's intraoral cavity (including teeth, gingival tissues, etc.). Such surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.), as was previously described. One of ordinary skill in the art, having the benefit of this disclosure, will appreciate that numerous methods, mechanisms, and strategies for generating an initial dental treatment plan exist, and that the discussed methods represent exemplary methods, mechanisms, and strategies for generating an initial dental treatment plan associated with the system.

In embodiments, an orthodontic treatment plan may be associated with any orthodontic procedure. Such a procedure may refer to, inter alia, any procedure involving the oral cavity and directed to the design, manufacture, or installation of orthodontic elements at a dental site within the oral cavity, or a real or virtual model thereof, or directed to the design and preparation of the dental site to receive such orthodontic elements. Such elements may be appliances including but not limited to brackets and wires, retainers, aligners, or functional appliances. In embodiments, various aligners may be formed for each treatment stage to provide forces to move the patient's teeth. The shape of such aligners may unique and customized for a particular patient and a particular treatment stage. Such aligners may each have teeth-receiving cavities that receive and resiliently reposition the teeth in accordance with a particular treatment stage.

In embodiments, each stage of the dental (e.g., palatal expansion and/or orthodontic) treatment plan may correspond to a specific set of aligners that the patient must wear for a predetermined period. In some embodiments, such a period, or time interval, may range from one to three weeks. For example, in some cases, orthodontic treatment may begin with the first set of aligners, tailored to fit a patient's current dental configuration. Such initial aligners apply targeted pressure on specific teeth, initiating the process of gradual realignment. Once the patient has worn this initial set of aligners for the duration specified in the first stage of the initial plan 248A, the patient may transition to a subsequent stage (e.g., the subsequent stage in a sequence of stages). This involves replacing the initial set of aligners with a new set, designed to continue the process of realignment. Subsequent stages may introduce a new set of aligners, manufactured to incrementally move the teeth closer to the desired final position.

As will be further described with respect to FIGS. 2B-C, in embodiments, such an initial treatment plan 248A may include checkpoints or assessment periods, where HCPs and/or dental professionals assess the progress of the treatment. During such checkpoints, digital scans, images, molds, etc., may be taken to ensure that the teeth are moving according to the planned trajectory.

In embodiments, such checkpoints, or assessments, may occur during or in between stages of a given dental treatment plan. In embodiments, the dental treatment plan may prescribe, or outline specific time intervals between checkpoints. In some embodiments, any of the previously discussed collected data types may be collected during such checkpoints.

As will be further discussed with respect to FIGS. 2B-C, if the dental treatment is progressing as or better than expected, aspects and parameters of the initial plan 248A may be updated. Additionally, if the treatment is not proceeding as expected, aspects and parameters of the initial treatment plan 248A may be updated or adjusted to better align with the patient's needs. Adjustments may be recorded and/or stored within the storage device as plan updates 248B. An example of such adjustments might include extending the duration of a stage, introducing new intermediary stages, revising the overall treatment plan, adjusting time intervals between checkpoints, introducing, or removing checkpoints, increasing the total number of checkpoints, etc.

In some embodiments, any, or all of data within storage device 244 may be accessed and modified by treatment coordination platform 220 (or other modules and platforms of the system), for further processing.

In some embodiments, any of the modules and or platforms can host or leverage an AI model (e.g., a local or externally accessible AI model, also referred to herein as a trained machine learning model) for decision making and/or processing associated with the respective module. For example, in embodiments, any of the control modules of platform 220, data processing platform 250, and/or management module 242 may be or incorporate AI model components to aid with managing aspects of the treatment plan coordination system (e.g., bandwidth requirements, data storage, network paths, etc.). For example, in embodiments, media systems 236A-B or client modules 232A-B may incorporate AI or other sophisticated models and algorithms to process, clean, filter, upscale, downscale, compress, sample, etc., data associated with the treatment plan coordination system, such as audio, image, video, text, tabular, or other data.

As will be further discussed with respect to FIG. 2B-4C, in some embodiments, optimization module may include an AI model to analyze collected data 246, and produce treatment plan updates 248B.

In embodiments, such an AI model may be a machine learning (ML) model such as one or more of decision trees (e.g., random forests), support vector machines, logistic regression, K-nearest neighbor (KNN), or other types of machine learning models, for example. In one embodiment, such an AI model may be one or more artificial neural networks (also referred to simply as a neural network). The artificial neural network may be, for example, a convolutional neural network (CNN) or a deep neural network.

In one embodiment, processing logic performs supervised machine learning to train the neural network.

In some embodiments, the artificial neural network(s) may generally include a feature representation component with a classifier or regression layers that map features to a target output space. A convolutional neural network (CNN), for example, may host multiple layers of convolutional filters. Pooling may be performed, and non-linearities may be addressed, at lower layers, on top of which a multi-layer perceptron is commonly appended, mapping top layer features extracted by the convolutional layers to decisions (e.g., classification outputs). The neural network may be a deep network with multiple hidden layers or a shallow network with zero or a few (e.g., 1-2) hidden layers. Deep learning is a class of machine learning algorithms that use a cascade of multiple layers of nonlinear processing units for feature extraction and transformation. Each successive layer uses the output from the previous layer as input. Neural networks may learn in a supervised (e.g., classification) and/or unsupervised (e.g., pattern analysis) manner. Some neural networks (e.g., such as certain deep neural networks) may include a hierarchy of layers, where the different layers learn different levels of representations that correspond to different levels of abstraction. In deep learning using such networks, each level may learn to transform its input data into a slightly more abstract and composite representation. In embodiments of such neural networks, such layers may not be hierarchically arranged (e.g., such neural networks may include structures that differ from a traditional layer-by-layer approach).

In some embodiments, such an AI model may be one or more recurrent neural networks (RNNs). An RNN is a type of neural network that includes a memory to enable the neural network to capture temporal dependencies. An RNN is able to learn input-output mappings that depend on both a current input and past inputs. The RNN will address past and future measurements and make predictions based on this continuous measurement information. One type of RNN that may be used is a long short-term memory (LSTM) neural network.

As indicated above, such an AI model may include one or more generative AI models, allowing for the generation of new and original content, such a generative AI model may include aspects of a transformer architecture, or a generative adversarial network (GAN) architecture. Such a generative AI model can use other machine learning models including an encoder-decoder architecture including one or more self-attention mechanisms, and one or more feed-forward mechanisms. In some embodiments, the generative AI model can include an encoder that can encode input textual data into a vector space representation; and a decoder that can reconstruct the data from the vector space, generating outputs with increased novelty and uniqueness. The self-attention mechanism can compute the importance of phrases or words within a text data with respect to all of the text data. A generative AI model can also utilize the previously discussed deep learning techniques, including recurrent neural networks (RNNs), convolutional neural networks (CNNs), or transformer networks. Further details regarding generative AI models are provided herein.

In some embodiments, storage device 244 may be hosted by one or more storage devices, such as main memory, magnetic or optical storage-based disks, tapes or hard drives, network-attached storage (NAS), storage area network (SAN), and so forth. In some embodiments, storage device 244 may be a network-attached file server, while in other embodiments, storage device 244 may be or may host some other type of persistent storage such as an object-oriented database, a relational database, and so forth.

In some embodiments, storage device(s) 244 may be hosted by any of the platforms or device associated with system 200 (e.g. treatment coordination platform 220). In other embodiments, storage device 244 may be on or hosted by one or more different machines coupled to the treatment coordination platform via network 202. In some cases, the storage device 244 may store portions of audio, video, image, or text data received from the client devices (e.g., client device 230A-B) and/or any platform and any of its associated modules.

In some embodiments, any one of the associated platforms (e.g., treatment plan coordination platform 220) may temporarily accumulate and store data until it is transferred to storage devices 244 for permanent storage.

It is appreciated that in some implementations, the functions of platforms 220 and/or 240 may be provided by a fewer number of machines. For example, in some implementations, functionalities of platforms 220 and/or 240 may be integrated into a single machine, while in other implementations, functionalities of platforms 220 and/or 240 may be integrated into multiple, or more, machines. In addition, in some implementations, only some platforms of the system may be integrated into a combined platform.

While the modules of each platform are described separately, it should be understood that the functionalities can be divided differently or integrated in various ways within the platform while still applying similar functionality for the system. Furthermore, each platform and associated modules can be implemented in various forms, such as standalone applications, web-based platforms, integrated systems within larger software suites, or dedicated hardware devices, just to name a few possible forms.

In general, functions described in embodiments as being performed by platforms 220, 240, and/or 250 may also be performed by client devices (e.g., client device 230A, client device 230B). In addition, the functionality attributed to a particular component may be performed by different or multiple components operating together. Platforms 220, 240, and/or 250 may also be accessed as a service provided to other systems or devices through appropriate application programming interfaces, and thus is not limited to use in websites.

It is appreciated that in some implementations, platforms 220, 240, and/or 250 or client devices of the system (e.g., client device 230A, client device 230B) and/or storage device 244, may each include an associated API, or mechanism for communicating with APIs. In such a way, any of the components of system 200 may support instructions and/or communication mechanisms that may be used to communicate data requests and formats of data to and from any other component of system 200, in addition to communicating with APIs external to the system (e.g., not shown in FIG. 2A).

In some embodiments of the disclosure, a “user” may be represented as a single individual. However, other implementations of the disclosure encompass a “user” being an entity controlled by a set of users and/or an automated source. For example, a set of individual users federated as a community in a social network may be considered a “user.” In another example, an automated consumer may be an automated ingestion pipeline, such as a topic channel.

In situations in which the systems, or components therein, discussed here collect personal information about users, or may make use of personal information, the users may be provided with an opportunity to control whether the system or components collect user information (e.g., information about a user's social network, social actions or activities, profession, a user's preferences, or a user's current location), or to control whether and/or how to receive content from the system or components that may be more relevant to the user. In addition, certain data may be treated in one or more ways before it is stored or used, so that personally identifiable information is removed. For example, a user's identity may be treated so that no personally identifiable information may be determined for the user, or a user's geographic location may be generalized where location information is obtained (such as to a city, ZIP code, or state level), so that a particular location of a user cannot be determined. Thus, the user may have control over how information is collected about the user and used by the system and components.

FIG. 2B illustrates an example optimization subprocess of the optimization module of FIG. 2A, according to some embodiments of the present disclosure.

In some embodiments, similar features, and components as were described in with respect to FIG. 2A may be used in the process 280A illustrated in FIG. 2B. For example, process 280A may leverage client device 260, client device 230B, collected data 246 (e.g., data collected from client device 260 and/or client device 230B), optimization module 252, and plan updates 248B. Such elements of FIG. 2B may correspond, or be similar, to client device 260, client device 230A or 230B, collected data 246, optimization module 252, and plan updates 248B, as seen and described with respect to FIG. 2A. Accordingly, such features and components as seen and described with respect to FIG. 2B may incorporate and augment at least the corresponding embodiments seen and described with respect to FIG. 2A.

As discussed with respect to FIG. 2A, collected data 246 may be collected from a patient via embedded, or non-embedded client devices. Collected data 246 may include any of the data discussed with respect to collected data 246 as seen and described with respect to FIG. 2A.

As discussed, optimization module 252 may perform an optimization operation (e.g., “Optimization 2.1” as referenced in FIG. 2B) and produce plan updates 248B from collected data 246. In embodiments, such plan updates 248B can include modifications made to the stages of an initial treatment plan, such as lengthening and/or shortening of one or more treatment stages. Such stages can be seen in an example treatment plan 280B as seen in FIG. 2C. Optimization module 252 may additionally or alternatively make other determinations and/or determine and/or perform other actions with respect to a dental treatment plan and/or with respect to a patient's dentition based on analysis of provided patient data in embodiments. For example, optimization module 252 may identify one or more oral health conditions and/or a severity of such identified oral health conditions, and perform one or more actions based on such oral health conditions. In another example, optimization module 252 may identify missing dental auxiliaries and perform one or more actions based on such missing dental auxiliaries. In another example, optimization module 252 may determine a level of wear on a dental appliance (e.g., on retainer), and perform one or more actions based on the level of wear. In another example, optimization module 252 may identify one or more teeth that have relapsed in position after palatal expansion treatment and/or orthodontic treatment, and perform one or more actions based on the relapsed teeth. Examples of actions that may be performed by optimization module 252 and/or based on an output of optimization module 252 include determining a treatment modification, generating a notification, outputting visualizations, increasing or decreasing a frequency with which photos of the patient's dentition are captured during the dental treatment plan, adjusting an assessment schedule, addressing one or more identified oral health conditions (e.g., by recommending restorative treatment), outputting patient recommendations, outputting doctor recommendation, scheduling an in-person doctor visit for a patient, recommending replacing one or more missing dental auxiliaries, ordering one or more dental appliances, simulating predicted future smile/faces/dentition of a patient, and so on.

Although illustrated separately, an understanding of plan 280B and the corresponding stages will be beneficial for understanding the process described in with respect to FIG. 2B. An explanation of such will now be provided.

FIG. 2C illustrates an example treatment plan 280B, according to some embodiments of the present disclosure.

In some embodiments, treatment plan 280B may be an example initial treatment plan, and may include separate stages 290A-D of the treatment plan. Initial treatment plans are discussed above with respect to FIG. 2A, and example initial treatment plan 280B may incorporate and augment at least the embodiments described therein. Accordingly, the four stages 290A-D seen in FIG. 2C should be seen as exemplary. One of ordinary skill in the art, having the benefit of this disclosure, will appreciate that numerous stages (e.g., more, or fewer stages than four) may be included in a treatment plan, including an initial treatment plan.

In embodiments, the initial treatment plan 280B may be crafted by an HCP such as an orthodontist or dentist specialized in orthodontic treatment and/or by treatment planning software. The design of this plan may take into account a multitude of factors, including but not limited to the current alignment of the patient's teeth, the complexity of the patient case, medical history, the desired end result, etc. Furthermore, specific goals and targets may be associated with each stage of the initial treatment plan, and a dental treatment plan in general. As discussed with respect to FIG. 2A, initial diagnostic tools such as X-rays, digital scans, physical molds, virtual 3D models, etc., may be employed by the HCP to generate an initial treatment plan to meet the unique needs of a patient. In alternate embodiments, the initial treatment plan may be entirely, or partially, computer-generated.

In embodiments, the initial dental treatment plan 280B may be divided into multiple stages, serving as a roadmap for the treatment. As an example, stages 290A-D may represent four sequential stages within plan 280B. Each stage may correspond to specific goals and targets in the treatment process and may be time-bound. For example, the length of each stage within 290A-D may vary depending on patient case details, treatment goals, patient physiology, methods of therapy, etc. For example, in a treatment plan for a tooth alignment process, each stage may involve a distinct set of aligners (or other dental appliance, e.g., palatal expanders, arch expanders, teeth whitening appliances, etc., depending on the treatment) to be worn for the duration of a stage. Similarly, in a treatment plan for palatal expansion, each stage may involve a distinct polymeric palatal expander to be worn on the upper dental arch. Although this disclosure focuses on orthodontic aligners and palatal expanders, the methods and systems disclosed herein apply to any suitable dental appliances and/or treatments (e.g., teeth whitening, orthorestorative treatments that include an orthodontic phase and a restorative phase) or non-dental medical appliances and/or treatments (e.g., bone healing, skin grafts, radiation therapy, chemotherapy) that include multiple stages requiring an active step and/or monitoring (e.g., by the patient, by an HCP, by an automated system) to advance to another (e.g., subsequent) stage. In embodiments, a length of each stage may be anywhere from 3 days, to 10 days, to 3 weeks, in duration.

In embodiments, each stage may be progressed from a first stage to a second stage (e.g., referencing FIG. 2C, from the stage 290A to the stage 290B) once a target or goal for tooth movement has been reached for the first stage. Thus, in embodiments each stage may be associated with a target or goal of the associated dental treatment plan. In embodiments, the duration of different stages may be set based on the difficulty of treatment, number of sequences, and/or strategies associated with such goals. Thus, in embodiments one or more of the stages may have different durations planned in the initial treatment plan. In alternate cases, duration for all stages may be uniform.

Continuing with the above example, initial treatment plan 280B may be oriented towards a tooth alignment process, involving the use of aligners that are worn by the patient. Additionally, or alternatively, initial treatment plan 280B may be oriented toward a palatal expansion process, involving the use of a sequence of palatal expanders to be worn by a patient in sequence. Such aligners and/or palatal expanders may exert specific forces that gradually move the teeth and/or palate into the desired position. Teeth movement and/or palatal expansion amount may be granularized, or discretized, into discrete stages corresponding to stages 290A-D.

For instance, stage 290A of the plan may correspond to an initial phase and a first set of aligners. During stage 290A, the primary objective may be to initiate the realignment process by targeting specific teeth that require the most immediate attention. This set of aligners may be worn for a predetermined period (e.g., from one to three weeks, as discussed above) after which the treatment progresses to the subsequent stage.

Stage 290B may serve as the second stage of the treatment plan, following the completion of stage 290A. In this stage, a new set of aligners (e.g., a new upper aligner and lower aligner) may be introduced, designed to build upon the tooth movement initiated in the first stage. In embodiments, aligners associated with stage 290B may be engineered to target a different set of teeth or to apply force vectors that are calibrated to further the goals set in the initial stage. As discussed above, in embodiments, the duration of this stage can vary based on treatment complexity but also may consider the overall length and stages of the total plan 280B to maintain a structured timeline.

The subsequent stages 290C and 290D may continue in a similar manner, further aligning teeth and introducing further refinements. In embodiments, the subsequent stages 290C-D (and any stages of a treatment process) may introduce further nuanced designs that apply specific torques or rotations to individual teeth. The final stage (e.g., 290D) may introduce final refinements for tooth alignment.

In some embodiments, a treatment may be a multi-phase treatment, each phase involving a different type of treatment. For example, referencing FIG. 2C, the initial treatment plan 280B may correspond to an orthorestorative treatment that includes an orthodontic treatment phase where a patient's teeth are aligned, and a subsequent restorative phase where a restorative such as a crown or veneer are applied to the patient's teeth. For example, stages 290A-D may correspond to stages of an orthodontic treatment phase, and a subsequent one or more stages (not shown) may correspond to a restorative treatment phase.

Returning to FIG. 2B, in light of the examples and embodiments provided by FIG. 2C, Optimization 2.1 may be an operation that modifies aspects of a treatment plan via plan updates 248B. This may include modifying an initial treatment plan, or a previously modified dental treatment plan.

In embodiments, optimization module 252 may modify a plan by introducing plan updates 248B. For instance, continuing with the aligner example discussed with respect to FIG. 2C, collected data 246 may be collected at plan checkpoints. As previously discussed with respect to FIGS. 2A-2B, plan checkpoints may be at points of transition between one stage to another, or during any of the stages. As discussed, in embodiments, data collection may be accomplished and gathered remotely, and unobtrusively (e.g., by the patient). Thus, such checkpoints and data collection may be hourly, daily, weekly, etc., in embodiments.

After data collection, in some cases during such a checkpoint, the optimization module 252 may analyze the collected data 246 to output an indication of a level of progression of a patient, and/or modifications to the treatment plan. For example, during a hypothetical checkpoint conducted between stages 290B and 290C, the optimization module 252 may collect data (e.g., real-time data) from the patient and output plan updates and/or progress indicators (progress indicators may be varied, and will be described below). Such outputs may be with respect to the current stage, and/or the treatment plan at large.

In embodiments, the collected data may include embedded (or explicit) progress indicators, or indications of a level of progression with respect to a stage of the treatment plan, or the treatment plan at large. In some embodiments, progress indicators may be determined based on analysis of the collected data. In an example, as discussed with respect to an orthodontic treatment plan, a set of dental appliances (e.g., aligners, palatal expanders, etc.) may correspond to the sequential treatment stages. The configuration of the dental appliances can be selected to elicit the tooth movements specified by the corresponding treatment stage and movement targets associated with each treatment stage. Collected data (including images, models, 3D data, etc.) can include progress indicators that can indicate whether or not, and how rapidly, one or more teeth, and a patient's overall dentition is progressing towards such movement targets, and/or progress indicators may be determined based on analysis of the collected data. In embodiments, such progress indicators may be compared to movement targets according to the schedule defined by the initial treatment plan, to determine a level of patient progression according to the treatment plan.

In some embodiments, progress indicators may be or include elements such as a patient's detention, a patient's dental arch, the position of one or more teeth with respect to the mouth, a tooth's position with respect to one or more teeth, differences in position and/or orientation of one or more teeth, differences in a palate width, differences in an arch length, differences regarding placement and type of dental auxiliaries, differences regarding bite position, differences regarding an occlusion surface, etc., where such differences may be between planned values and measured values. Additional progress indicators can include structural elements including whether a dental auxiliary is not attached to a tooth as specified in the treatment plan, whether a dental auxiliary on a tooth is in an incorrect location or has an incorrect shape, whether a planned amount of interproximal reduction (IPR) was performed, etc. Additional progress indicators can be, include, or be extracted from any oral health elements or data captured (as were discussed with respect to FIG. 2A) including oral temperature data, oral pH data, pressure data (e.g., bite force or pressure otherwise sensed by a specific tooth, etc.), image data (including x-ray, or ultrasound, color images, etc.), biomarker data, heart rate data, respiratory data, body temperature, and/or raw data indicative of electrical parameters associated with health data (e.g., capacitance, resistance, conductance values, etc. gathered from a dental appliance), etc.

From a generalized viewpoint, such progress indicators can serve as tools to characterize: if movement of a tooth is happening, what phase of tooth movement the patient is experiencing, and/or the rate of tooth movement, etc.

In embodiments, such progress indicators can be compared to historical data (e.g., collected data) taken from a prior checkpoint to determine a level of progression. In alternate embodiments, the current level of progression may be determined by comparing one or more of the progress indicators to projected or predicted targets and goals. For example, in some embodiments, a progress indicator may be or include the current condition of the patient's dental arch. Such a condition and progress indicator may be included in image data (e.g., included within collected data 246A) that was generated based on an intraoral scan of the patient's dental arch. In embodiments, such a comparison can be made by extracting the condition of the dental arch from the collected data, e.g., by forming a 3D model, or otherwise characterizing the patient's dental arch. Such a characterization of the dental arch can then be compared to similar data forms indicating the targeted condition. For example, in some cases, a 3D model of the patient's dental arch formed from the patient's collected data may be compared to a predicted/target 3D model of the target dental arch including in the treatment plan. In such a case, the comparison can produce deviations, or characterize a level of progression, for the current treatment stage and the multi-stage orthodontic treatment plan at large.

Based on such comparison of the image data and extracted progress indicators to the targeted or predicted conditions for a given treatment stage, optimization module 252 may determine any signs of deviation. The optimization module 252 may then determine any modifications for the dental treatment plan necessary for achieving the treatment goals. For example, if such a comparison indicates that the patient treatment is behind schedule, the current stage may be prolonged. This may include continuing with the current set of aligners for additional time than originally planned for in the treatment plan. If such a comparison indicates the patient treatment is advancing more rapidly than expected, in some cases the patient may be advanced to the subsequent treatment stage earlier than planned. In such a way, progress indicators from within the patient data may be used to determine a patient's level of progression with respect to a treatment stage, and a dental treatment plan at large.

In some embodiments, the functions of optimization module 252 may be accomplished by any combination of human and machine components. For example, in some embodiments, a software program may process the collected data to extract the progress indicators into a different form (e.g., a 3D model that is formed from image data, a “heat map” characterizing tooth movement, etc.) and then a human (e.g., an HCP) may produce plan updates from the progress indicators. In further embodiments, a machine-made judgement (e.g., treatment plan updates) and the associated data may be presented to an HCP to simply confirm, or reject, the machine-made judgement or assessment. In embodiments, any mixture of human and software elements may be combined. Such an optimization module may thus leverage strengths from both human and machine capabilities.

In some embodiments, the machine element of the treatment decision making process may be foregone, and an HCP may be directly shown the collected data 246. The HCP may solely assess and judge whether the patient should advance to the subsequent stage, or remain at the current stage for additional time.

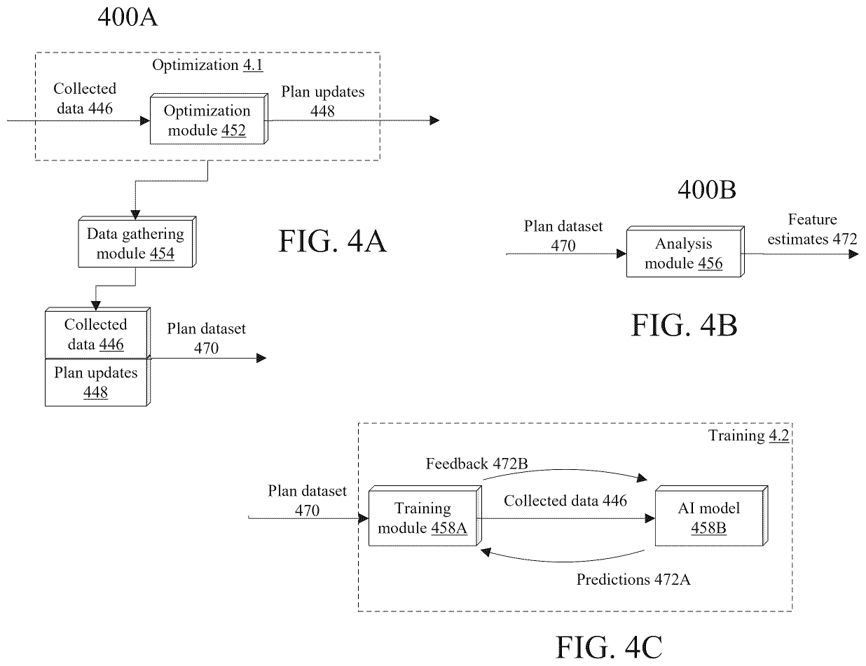

As was discussed with respect to FIG. 2A, and as will be further described with respect to FIG. 4C, in embodiments, the plan updates may be produced directly from the progress indicators by a machine-learning (or similar) model. In such embodiments, the optimization module 252 may be completely automated, and performed by any kind of computer algorithm (e.g., a machine-learning model, an analytical model, a loss-optimization function, etc.).

Thus, in some embodiments, optimization module 252 may be, or include, a visual transformer, a machine-learning model, a convolutional neural network, etc. (in addition to the embodiments discussed with respect to FIG. 2A). Through such a program and/or computational model, the optimization module 252 may determine a patient's level of progression, and whether the patient has progressed enough to transition to the subsequent stage of treatment. If the level of progression indicates that the patient is ready to advance to a subsequent stage, such a stage may be initiated (in embodiments, by the machine-learning model). If the level of progression indicates that the patient is not ready to advance to a subsequent stage of treatment, the current stage may be prolonged. As such, the optimization module may further personalize, and optimize the planned, or initial, or initial treatment plan.

In some embodiments, the one or more progress indicators are identified by processing patient data using a trained machine learning (ML) model that determines the one or more progress indicators and outputs data on the determined progress indicator(s). Output data may include values or metrics on the progress indicators, segmentation information and/or bounding boxes around objects that are, or are associated with, progress indicators in input images, and so on. In some embodiment, a level of progress of a dental treatment plan is determined using a trained ML model. The patient data and/or determined progress indicators may be input into a trained ML model, which may output information on an estimate level of progress in some embodiments. In some embodiments, a same ML model may determine progress indicators and a level of progress.

Given that rapidity and non-invasiveness associated with remote data collection, in embodiments the Optimization 2.1 (described above with respect to FIG. 2B) may be run at any routine or predefined schedule. This may include any feasible level of granularity (e.g., hourly, weekly, etc., as previously discussed) with respect to the overall treatment strategy. Thus, the optimization module 252 may continuously provide updates to further personalize and optimize the plan for a specific patient. In some embodiments, the frequency with which to gather updated patient data and assess a patient's treatment progress based on the patient data may be updated by optimization module 252. For example, if the patient is responding to treatment faster than anticipated and will be advanced to a subsequent treatment stage earlier than anticipated, then optimization module 252 may recommend more frequent collection of patient data. For instance, an initial plan may include observing and tracking checkpoint schedules that may or may not have been personalized for the patient based on patient data (e.g., such as patient demographics including age, gender, medical history, and other relevant characteristics). In embodiments, these checkpoint schedules may be modified and updated dynamically based on the patient's response to the treatment, adherence to prescribed routines, and other relevant indicators of progress (as has been discussed with respect to progress indicators and any other collected data described with respect to FIGS. 2A-2C).

Thus, the optimization module 252 may allow a patient to accelerate, or skip stages, while prolonging stages when prompted by the collected data, and further personalize a treatment plan. Thus, the length of each stage of a treatment plan may be optimized, or personalized, with further precision and granularity. In embodiments, this may save a patient, and an HCP, valuable time, and resources, and overall increase the effectiveness of a treatment plan.

In some embodiments, the rationale and/or process for arriving at a plan update and/or level of progression may be provided to the patient, as a method of incentivization for the patient to increase or maintain compliance (e.g., to wear aligners longer). In an example, in cases where a plan update of plan updates 248B is to retain the patient at the current stage, either due to non-compliance or some other factor, such underlying factors may be presented to the patient. Such a presentation may function to link patient behaviors, biology, genetics, etc., to the plan updates or outcomes. For example, in a case where a patient has low levels of compliance with respect to their treatment plan, compliance data indicating such may be presented to the patient to “link” such behavior to the outcome, and further incentivize the patient to enhance their rate of compliance. Similarly, if added efforts or actions of the patient are resulting in accelerated or quickened treatment, such a positive link may be presented to the patient so as to further incentivize such positive behaviors. Thus, through such transparency, the optimization process and system at large may incorporate an additional factor for increasing efficiency of the treatment plan.

FIG. 3 illustrates an example optimization subprocess of the optimization module of FIG. 2A, in accordance with some embodiments of the present disclosure.

In some embodiments, similar features and components as were described in with respect to FIGS. 2A-2C may be used in the process 300 illustrated in FIG. 3. For example, process 300 may correspond or be similar to process 280A, as seen and described with respect to FIG. 2B. Accordingly, process 300, as seen and described with respect to FIG. 3, may incorporate and augment at least the corresponding embodiments seen and described with respect to FIG. 2B. In embodiments, process 300 may include a treatment plan including stages 306A-D (or greater or fewer stages). Such stages of FIG. 3 may correspond, or be similar, to stages 290A-D, as seen and described with respect to FIG. 2C. Accordingly, such stages as seen and described with respect to FIG. 3, may incorporate and augment at least the corresponding embodiments seen and described with respect to FIG. 2C.

In embodiments, a remote assessment 310 may be accomplished to collect patient data. Such patient data may correspond, or be similar, to collected data 246 as seen and described in FIGS. 2A and 2B and incorporate and augment at least the embodiments described therein. At operation assessment schedule 312, such data collected by remote assessment 310 may be combined with compliance data 314, to generate an updated assessment schedule 312. Such an updated assessment schedule 312 may correspond, or be similar, to plan updates 248B as seen and described in FIGS. 2A and 2B, and incorporate and augment at least the embodiments described therein. Thus, the updated assessment schedule 312 may be utilized to update the length, or any other aspect of, the treatment plan including stages 306A-D.

In some embodiments, compliance data 314 may be an indicator of the amount of time, or level of adherence, that a patient is complying with a prescribed treatment plan. For instance, in the case of aligners and/or palatal expanders, if a minimum wear time is prescribed of 12 hours a day for 10 days, compliance data 314 may include (and have tracked) the total daily amounts of wear time, for the prescribed period of time (e.g., 10 days). Thus, an HCP or computer module may review the compliance data and incorporate such data into modifications made to a treatment plan. E.g., in embodiments, such compliance data may be included in collected data 246 as seen and described in FIGS. 2A and 2B.

In embodiments, data produced by the remote assessment 310 (which may or may not include compliance data, in embodiments), may be further used in a stage verification operation 316. In embodiments, stage verification 316 may correspond, or be similar to Optimization 2.1, as seen and described in FIG. 2B, and incorporate and augment at least the embodiments described therein. In embodiments, stage verification 316 may judge or assess whether the collected data from the remote assessment indicates the patient has sufficiently progressed for stage advancement (at operation 318), or if the patient is to remain at the current stage. In embodiments, the time between the current remote assessment, and the subsequent scheduled remote assessment, may be modified, or scheduled, based on the collected data from the remote assessment 310.

FIG. 4A illustrates an example data gathering subprocess performed by the data management module of FIG. 2A, in accordance with some embodiments of the present disclosure.

In some embodiments, similar features, and components as were described in with respect to FIGS. 2A-3 may be used in the process 400A illustrated in FIG. 4A. For example, optimization 4.1 may correspond, or be similar to, optimization 2.1 as seen and described with respect to FIG. 2B. Accordingly, optimization 4.1, as seen and described with respect to FIG. 4A, may incorporate and augment at least the corresponding embodiments seen and described with respect to FIG. 2B. In embodiments, process 400A may further include data gathering module 454, collected data 446, an optimization module 452, and plan updates 448. Such elements of FIG. 4A may correspond, or be similar, to data gathering module 254, collected data 246, optimization module 252, and plan updates 248B, as seen and described with respect to FIGS. 2A-2B. Accordingly, such features and components as seen and described with respect to FIG. 4A, may incorporate and augment at least the corresponding embodiments seen and described with respect to FIGS. 2A-2B.

In some embodiments, data gathering subprocess 400A may be performed by the data gathering module 454 and may build on top of systems and methods as described with respect to Optimization 2.1 in FIG. 2B. For example, data gathering subprocess 400A may intake the collected data 446 and plan updates 448 (which may be similar or analogous to similar elements in FIGS. 2A-2B and incorporate the embodiments discussed therein), and generate a plan dataset 470, which incorporates the collected data 446 as inputs, and the plan updates 448 as correlated outputs.

In embodiments, the plan dataset 470 may aggregate data from multiple patients and multiple sampling times. For example, in embodiments, plan dataset 470 may include collected data 446 from a patient taken from multiple stages along that given patient's treatment plan. In embodiments, plan dataset 470 may contain collected data 446 from multiple (including any number of) patients as well. In embodiments, the data may be discretized according to data segments including related collected data and plan updates, corresponding to a linked dental procedure, checkpoint and/or treatment stage, and/or patient. In embodiments where plan dataset 470 includes various types of data, data segments of plan dataset 470 may be categorizable by dental procedure type (e.g., specific tooth manipulations, specific location of procedure within the mouth, etc.), patient type and/or patient characteristics (e.g., age, demographics, etc.), etc.

In alternate embodiments, the data gathering subprocess 400A may be performed by other modules of the system (e.g., data management module 226, or other modules such as analysis module 256) as seen and described in FIG. 2A, in addition to their functionalities described therein.

In embodiments, the plan dataset 470 may contain data that corresponds to a specific dental procedure (including any of the orthodontic, restorative, palatal expansion and/or other dental procedures heretofore discussed). In addition, plan dataset 470 may correspond to a procedure including an orthodontic procedure, a dental alignment procedure, a gingivectomy procedure, a bone or gum graft, a temporomandibular joint (TMJ) treatment, a palatal expansion procedure, an alveoloplasty procedure, a glossectomy procedure, an orthognathic procedure, a biopsy, a vestibuloplasty procedure, a restorative dental procedure, etc.

In embodiments, plan dataset 470 may contain one or more data segments of data that corresponds to an even further granularized, more specific portion of a dental procedure. For example, within a dental produce, in embodiments, the plan dataset may contain a data segment (i.e., collected data and plan updates) that corresponds to any tooth or oral element, including (maxillary or mandibular), incisors (central or lateral), canines, premolars (first or second), molars (first, second, or third), any portion of the gums, the jawbone, the palate, glands, sinuses, or nerves, etc.

In embodiments, e.g., within an orthodontic procedure, the plan data may correspond to any movement or manipulation of any tooth, including tipping, torquing, intrusion, extrusion, rotation, bodily movement (e.g., mesial, distal, vertical, etc.), retraction, protraction, space closure, space opening, etc.

In some embodiments where plan dataset 470 has been further granularized, process 400A may produce multiple, including any number of, plan dataset(s) 470, corresponding to multiple, or any number of specific procedures.

In some embodiments, subprocess 400A may output the plan data 470 for further processing. In embodiments, such further processing may include advanced data analysis, training of a machine learning module, segmentation, or modeling of the data, etc.

FIG. 4B illustrates an example data analysis subprocess performed by the analysis module of FIG. 2A, in accordance with some embodiments of the present disclosure.

In some embodiments, similar features, and components as were described in with respect to FIGS. 2A-4A may be used in the process 400B illustrated in FIG. 4B. For example, in embodiments, process 400B may further include analysis module 456 and plan dataset 470. Such elements of FIG. 4B may correspond, or be similar, to analysis module 256, and plan dataset 470 as seen and described with respect to FIGS. 2A and 4A. Accordingly, such features and components as seen and described with respect to FIG. 4B, may incorporate and augment at least the corresponding embodiments seen and described with respect to FIGS. 2A and 4A.

Analysis module 456 may perform a data analysis subprocess, to extract insights and feature estimates from a plan dataset 470. In some embodiments, feature estimates may be extracted and correlate to procedures according to varying levels of granularity, ranging from an overall holistic procedure such as general dentition alignment, to a specific procedure for manipulating a single, specific tooth.

To accomplish such functions, plan dataset 470 may be modified, and relevant data extracted before input to analysis module 456. In embodiments where plan dataset 470 corresponds to a single procedure, or where multiple datasets are generated each corresponding to a single procedure, the data may be analyzed by module 456 to further characterize the procedure without modification. In cases where multiple procedures are contained within a stored plan dataset, the data segments may be categorized, and data segments corresponding to a single procedure may be extracted for processing.

In embodiments, where further levels of granularity are desired, the plan dataset may be categorized and the plan dataset can be “focused,” to retain data segments corresponding to the desired level of granularity. For example, within a general orthodontic procedure, aligners may be designed to apply specific manipulations to one or more teeth. These manipulations can include, tipping, torquing, rotating, extruding, retracting, protracting, etc., as listed above. In embodiments, plan dataset 470 can be focused (e.g., only include the data segments) to include only collected data and plan updates for such a single manipulation, for a single tooth, and the produced feature estimates 472 can correspond to such a specific dental procedure.

Accordingly, in embodiments, the plan data may be aggregated and analyzed to produce feature estimates 472, e.g., generalizations, characterizations, estimates, or insights, etc., based on the data for a specific procedure, manipulation, or element, as described above. For example, in embodiments in which plan dataset 470 is manipulated to contain data which corresponds to a bodily movement of a specific tooth, plan dataset 470 may be processed and analyzed to generate feature estimates and characterizations for that bodily movement. Such estimates and characterizations can include features such as estimated average procedure duration, estimated average procedure difficulty, identification of subprocesses that may be required for the overall procedure, etc. In embodiments, such estimates and characterizations may be generated for a given bodily movement, a given tooth, a given patient data (e.g., biology, age, etc.), or any other influencing factor that has been captured within collected data within the plan dataset, according to any level of granularity.

In some embodiments, feature estimates 472 may be based on a wide array of qualitative and quantitative input metrics (e.g., parameters within collected data 446). For instance, the level of difficulty of the procedure may be estimated or assessed based on parameters such as the degree of tooth misalignment, the type of manipulation needed (e.g., tipping, torquing, rotation, etc.), the number of teeth involved, the general oral health condition of the patient, the recorded amount of plan updates that were required and their nature, patient compliance, etc. In some embodiments, a dental treatment plan may be generated that includes multiple stages. Each of the treatment stages may be configured to perform one or more specific tooth movements. Analysis module 456 may determine a difficulty or challenge level of tooth movements associated with individual stages of treatment in some embodiments. Accordingly, in addition to an overall treatment plan being assigned a level of difficulty, individual treatment stages of the treatment plan may additionally be assigned their own individual levels of difficulty (e.g., challenge level). In embodiments, for example, the estimated length of time for the procedure may be calculated based on historical data from similar cases, the complexity of similar manipulations, the historical responsiveness of individual teeth to orthodontic forces for such a procedure, etc.

Furthermore, in embodiments, plan dataset 470 may also include data related to the estimated patient discomfort or pain levels, anticipated need for adjunctive procedures (e.g., extractions, bone grafts, etc.), as well as the expected aesthetic outcomes.

In some embodiments, such feature estimates and characterizations may be dynamically updated to reflect real-time progress, facilitating more accurate planning and adjustment of ongoing treatments.

In some embodiments, the generation of feature estimates 472, and the functions of analysis module 456 may be accomplished by a human, a computer or software module, or any combination of the two. For instance, in embodiments, an HCP or similarly trained dental professional may analyze the plan dataset to generate feature estimates or generalization for a given procedure. In alternate embodiments, a software program, mathematical model, or any similar computer program may accomplish the functions of analysis module 456.

FIG. 4C illustrates an example AI model 458B usable, for example, for the analysis module of the system of FIG. 2, in accordance with some embodiments of the present disclosure.

In some embodiments, similar features, and components as were described in with respect to FIGS. 2A-4B may illustrated in FIG. 4C. For example, in embodiments, FIG. 4C may include plan dataset 470 and collected data 446. Such elements of FIG. 4C may correspond, or be similar, to plan dataset 470 and collected data 246 and/or 446 as seen and described with respect to FIGS. 2A-4B. Accordingly, such features and components as seen and described with respect to FIG. 4C, may incorporate and augment at least the corresponding embodiments seen and described with respect to FIGS. 2A-4B.

In some embodiments, optimization module 452 and/or 252 (as were seen in FIGS. 2A, 2 and 4A) may be, include, or leverage an AI model 458B (also referred to as an ML model herein) and/or a training module 458A as currently described. In embodiments, the AI model 458B may be trained to generate plan updates 448 and/or new plans as were seen and discussed with respect to FIG. 4A (and 248B in FIG. 2A-2B) from collected data 446. In some embodiments, AI model 458B may be trained to perform other functions, such as identifying progress indicators of treatment progress with respect to a dental treatment plan, determining a level of progress associated with a dental treatment plan, identifying oral health conditions (e.g., gingival inflammation, tooth chips, tooth cracks, missing teeth, caries, etc.), determining severity of identified oral health conditions, identifying missing dental auxiliaries, determining a level of wear of a dental appliance, identifying relapsed teeth, generating synthetic images of dentition and/or patient smiles at one or more future stages of treatment, and so on. In embodiments, the AI model 458B may be any kind of AI model or machine learning model as has been previously discussed with respect to FIG. 2A.

In some embodiments, once sufficient data has been gathered (i.e., once plan dataset 470 becomes large enough), the AI model 458B can be trained using plan dataset 470. In some embodiments, the AI model 458B may be trained for a specific type of dental procedure, according to varying levels of granularity. In some embodiments, the training process seen in FIG. 4C may be repeatable for varying types of procedures, and/or more than one trained AI model may be used to accomplish the functions of the optimization module of the system.

In embodiments, a large corpus of training data including plan dataset 470 and/or collected data 446 may be used to train the AI model 458B. The plan dataset 470 may include historical data of many treatment plans that were applied to treat patients, and collected data 446 may include progress data (e.g., patient data) showing how treatment progressed for each of those patients and/or treatment plans. Collected data may additionally or alternatively include labeled image data including labels of progress indicators of treatment progress with respect to a dental treatment plan, labels of a level of progress associated with a dental treatment plan, labels for oral health conditions (e.g., gingival inflammation, tooth chips, tooth cracks, missing teeth, caries, etc.), labels for severity of identified oral health conditions, labels for missing dental auxiliaries, labels indicating a level of wear of a dental appliance, labels identifying relapsed teeth, and so on. Based on such data, AI model 458B may be trained to receive patient information (e.g., a 3D model of a patient's current upper and lower dental arches, patient age, patient gender, patient demographics information, 2D image(s) of a patient's dentition, smile, face, etc.) and output a new treatment plan prior to treatment or during treatment, and/or to output other information such as progress indicators of treatment progress with respect to a dental treatment plan, a level of progress associated with a dental treatment plan, oral health conditions (e.g., gingival inflammation, tooth chips, tooth cracks, missing teeth, caries, etc.), severity of identified oral health conditions, indications of missing dental auxiliaries, a level of wear of a dental appliance, identifications of relapsed teeth, and so on.

In one embodiment, because the AI model 458B has been trained on data showing not only treatment plans for patients and final outcomes for those patients, but also intermediate outcomes for those patients during various stages of treatment, treatment plans output by the AI model 458B may have increased accuracy. Additionally, or alternatively, AI model 458B may be trained to generate recommendations for existing treatment plans and/or updates to existing treatment plans based on an input of an already generated treatment plan and/or patient data gathered at a current stage of treatment and/or prior stages of treatment for the treatment plan. The AI model 458B may output changes to lengths of treatment for one or more stages of treatment, may add additional stages of treatment, may remove stages of treatment, may otherwise modify existing stages of treatment, and so on.

In addition to the functions of optimization module 452, the AI model 458B may be trained to perform the functions, or similar functions of analysis module 456. For example, model 458B may be trained to perform any of the estimates or characterizations described above, including summarization of data related to one or more procedures, production of estimates for procedure duration, difficulty, required subprocess, etc. In embodiments, such estimates may be generated by model 458B for a given set of patient characteristics (e.g., age, sex, genetical information, etc.)

Continuing to embodiments where the AI model 458B is trained to generate plan updates, operation training 4.2 may be used for generating a trained AI model 458B that may generate plan updates from collected data 446. In embodiments, the AI model may further be trained to generate data features 472 from plan dataset 470, as mentioned.

In some embodiments, the collected data 446 may be the initial data points or stimuli that are fed into the AI model 458B. These inputs may be any of the data previously discussed with respect to collected data (e.g., with respect to FIGS. 2A-4A) corresponding to a single procedure.

Complementing the inputs, in embodiments, the plan updates and/or estimated features 472 may be outputs that the AI model 458B attempts to correctly predict (e.g., via predictions 472A).

In some embodiments, the training module 458A may access the plan dataset 470, deliver collected data 446 to AI model 458B as inputs, and may iteratively provide feedback 472B to the predictions 472A generated by the AI model 458B. For example, for each iteration of training, an instance, or data point, from collected data 446 may be fed as input into the AI model, prompting the AI model to produce an output. This output (e.g., the predicted plan updates and/or data analytics corresponding to such an input) may then be compared against the correct output from plan dataset 470. Any discrepancies between the model's output and the correct output may be noted, and feedback may be provided to the model to minimize such discrepancies in future iterations.

Such an iterative, continuous feedback loop may continue until a certain level of accuracy is reached by the prediction of the AI model 458B.

In embodiments, after training, the AI model 458B may be tested on a validation dataset, to ensure accuracy level are sufficient to continue deploy the model within or as a replacement to an optimization module of the system.

In some embodiments, such an AI model 458B may vastly increase the efficiency of the system, and the production of plan updates and/or data analytics. For example, in some embodiments, the AI model, may be a comprehensive and intricate AI model that has been extensively trained on vast and diverse data. Accordingly, the AI model may possess broad knowledge and understanding of patient data including images, sensor data, text data, and any other types of data within collected data within the plan dataset 470.

Thus, in embodiments, the process of data collection, data gathering and categorization, data analysis, and treatment plan optimization, as seen and described within FIGS. 2A-4B, may be entirely automated.

In some embodiments, such a system as seen and described within FIGS. 2A-4B may be employed as a vehicle for testing different treatment plans, so as to verify a strategy and/or treatment plan from a variety of different treatment plans that achieves more desirable outcomes. For instance, a treatment plan could vary from a separate treatment plan through use of unique elements, components, or overall strategies. For instance, a single treatment plan could differ by incorporating different aligner materials, a different order of teeth movement, varying duration and/or checkpoint schedules, adherence to different schools of thought (e.g., strategies) on the order of teeth movement, etc. Thus, in some embodiments, the system could be used as a tool to verify, validate, and/or compare competing or novel strategies, components, elements, and materials associated with different treatment plans.

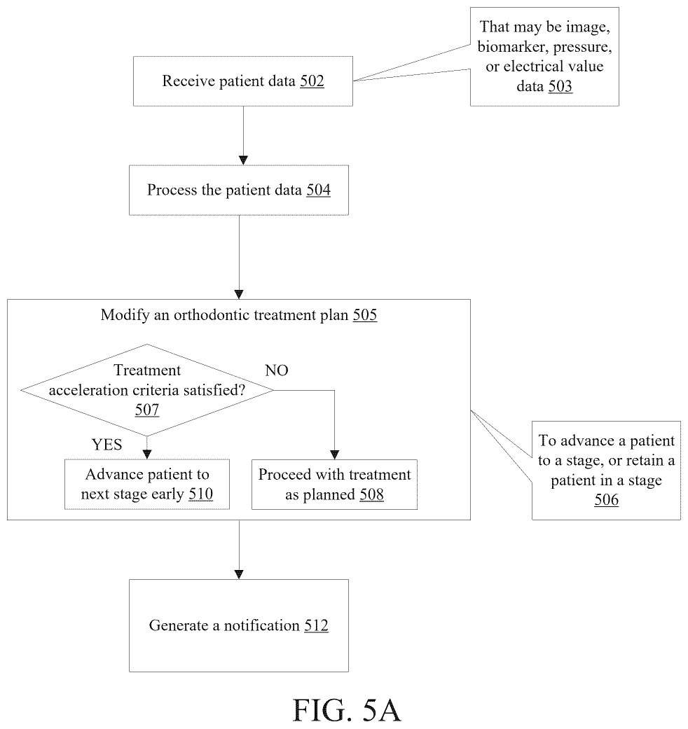

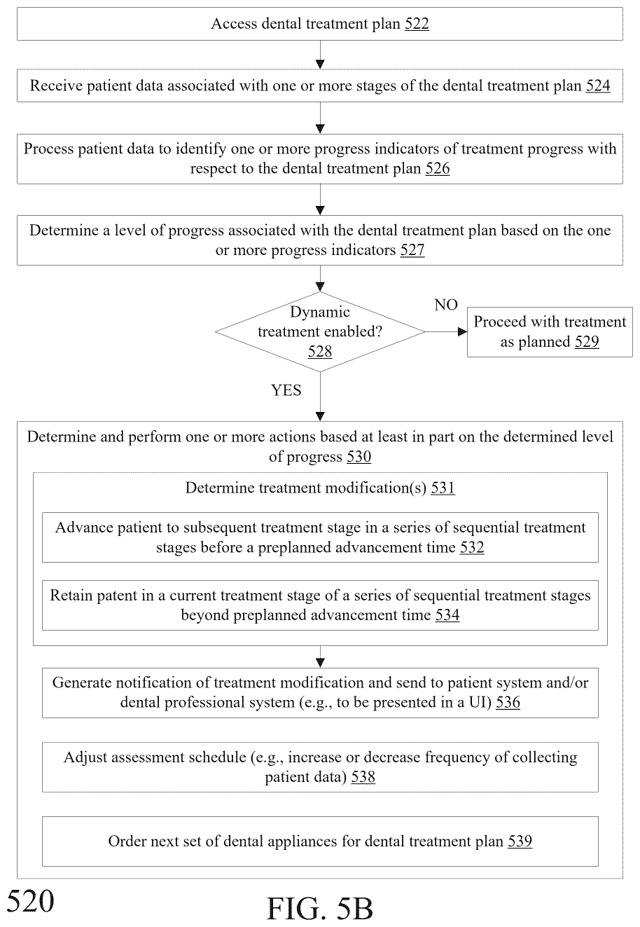

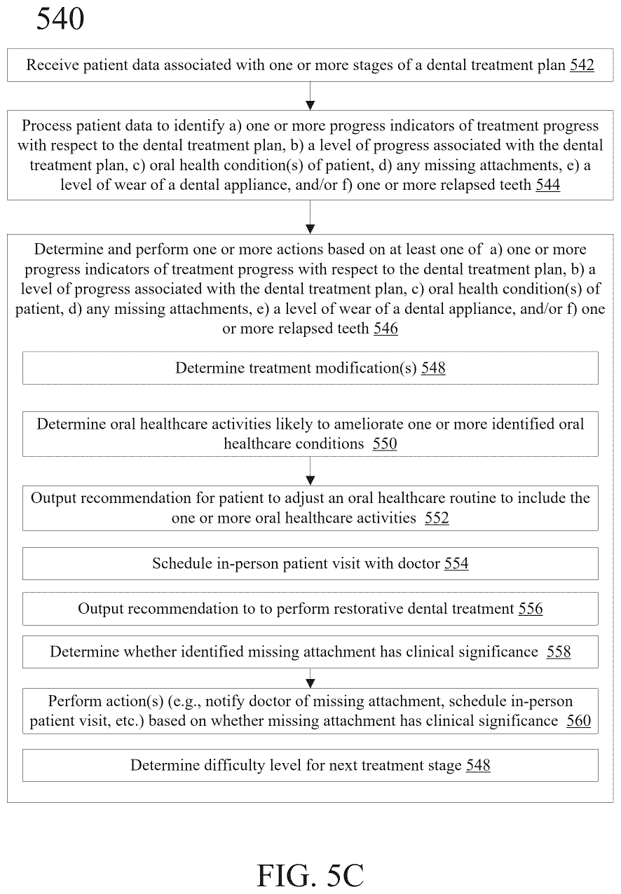

FIGS. 5A-C illustrate flow diagram of example methods for assessing a patient dentition, modifying a dental treatment plan and/or performing other action with respect to dental treatment, in accordance with some embodiments of the present disclosure.

The methods may be performed by a processing device that may include hardware, software, or a combination of both. The processing device may include one or more central processing units (CPUs), graphics processing units (GPUs), field-programmable gate arrays (FPGAs), application-specific integrated circuits (ASICs), or the like, or any combination thereof. In one embodiment, one or more of the methods may be performed by the processing devices and the associated algorithms, e.g., as described in conjunction with FIGS. 2A-2B. In certain embodiments, one or more of the methods may be performed by a single processing thread. Alternatively, one or more of the methods may be performed by two or more processing threads, each thread executing one or more individual functions, routines, subroutines, or operations of one or more methods. In an illustrative example, the processing threads implementing one or more methods may be synchronized (e.g., using semaphores, critical sections, and/or other thread synchronization mechanisms). Alternatively, the processing threads implementing one or more methods may be executed asynchronously with respect to each other. Therefore, while FIGS. 5A-C and the associated descriptions list the operations of the methods in a certain order, in some embodiments, at least some of the described operations may be performed in parallel and/or in a different order. In some embodiments one or more operations of is the methods may not be performed.

FIG. 5A illustrates a flow diagram of an example method 500 for modifying an orthodontic treatment plan (e.g., via the optimization module of FIG. 2A), in accordance with some embodiments of the present disclosure.

At block 502, method 500 may include receiving patient data. In some embodiments, receiving patient data may include receiving patient data comprising one or more progress indicators associated with an orthodontic treatment plan.

As illustrated with callout block 503, the processing device performing method 500 may receive patient data that includes image, biomarker, pressure, and/or electrical value data. In some cases, receiving patient data that includes image, biomarker, pressure, and/or electrical value data may include receiving patient data that includes at least one of image data of a patient's dentition, biomarker data indicative of changes in the patient's dentition, pressure data indicative of a level of pressure exerted by the patient's dentition on an orthodontic aligner, or data indicative of an electrical parameter associated with a position of a tooth with respect to an orthodontic aligner.

At block 504, method 500 may include processing the patient data. In some embodiments, processing the patient data may include processing the patient data to determine a level of progression associated with the orthodontic treatment plan based on the one or more progress indicators.

At block 505, method 500 may include modifying an orthodontic treatment plan. In some embodiments, modifying an orthodontic treatment plan may include modifying the orthodontic treatment plan in response to the determined level of progression.

As illustrated with callout block 506, the processing device performing method 500 may modify an orthodontic treatment plan to advance a patient to a stage, or retain a patient in a stage. In some cases, modifying an orthodontic treatment plan to advance a patient to a stage, or retain a patient in a stage may include modifying the orthodontic treatment plan in response to the determined level of progression comprises advancing a patient associated with the patient data to a subsequent stage of the orthodontic treatment plan or retaining the patient within a current stage of the orthodontic treatment plan.

In one embodiment, at block 507 processing logic determines whether treatment acceleration criteria are satisfied. If treatment acceleration criteria are satisfied, then a treatment plan may be accelerated by progressing to one or more next treatment stages earlier than initially planned. In one embodiment, a first treatment acceleration criterion is selection of an option to enable treatment acceleration by a doctor. For example, a UI may be presented to a device of a doctor that includes one or more treatment options. One of the treatment options may be to enable automated treatment acceleration and/or to otherwise enable automated treatment modification. The doctor may select enablement of such an option to turn on the automated treatment modification or acceleration feature in embodiments. If treatment acceleration is enabled, then processing logic may determine whether one or more other treatment acceleration criteria have been satisfied. One criterion may be that a patient has been compliant in wearing dental appliances. Compliance may be determined based on, for example, sensor data indicating that the patient is wearing the dental appliance as prescribed, self-reported data, etc. Another criterion may be that a threshold number of prior patient assessments from an assessment schedule (each separated by a predefined time interval) indicated that treatment was progressing as planned or faster than planned (as determined based on, e.g., analyzing photos from the patient and comparing actual tooth positions to planned tooth positions, determining a level of fit of a dental appliance over a tooth by measuring a gap between the dental appliance and the tooth). Another criterion may be that a current assessment of the patient data indicates that treatment advancement to a next treatment stage early is recommended. Another criterion may be that a next treatment stage is not a complex treatment stage (e.g., has a difficulty level that is below a difficulty threshold). If not all treatment acceleration criteria are satisfied, the method may proceed to block 508 and treatment may proceed as a planned, even if an output of processing the patient data was an indication that the patient could advance to a next treatment stage earlier than originally planned. If all treatment acceleration criteria are satisfied, the method may continue to block 510, and a patient may be advanced to a next treatment stage early (shortening a current treatment stage) and/or a length of the next treatment stage may be reduced (shortening the next treatment stage).

At block 512, method 500 may include generating a notification. In some embodiments, generating a notification may include generating a notification of the modified orthodontic treatment plan.

FIG. 5B illustrates a flow diagram of an example method 520 for modifying an orthodontic treatment plan, in accordance with some embodiments of the present disclosure. At block 522 of method 520, processing logic accesses a dental treatment plan and/or otherwise receives the dental treatment plan and/or information from the dental treatment plan. At block 524, processing logic receives patient data associated with one or more stages of the dental treatment plan. The patient data may include any of the types of patient data discussed herein, such as images of a patient's dentition, face, smile, etc. Other patient data may include, for example, data of a dentition of the patient, biomarker data indicative of changes in the dentition, pressure data indicative of a level of pressure exerted by the dentition on a dental appliance, data indicative of an electrical parameter associated with a position of a tooth with respect to a dental appliance, and so on. The patient data may be generated without a need for the patient to visit a healthcare provider in embodiments.

At block 526, processing logic processes the patient data to identify one or more progress indicators of treatment progress with respect to the treatment plan. As discussed above, many different types of progress indicators may be identified, such one or more tooth positions, one or more tooth orientations, arch width, and so on. The one or more progress indicators comprises, for example, indicators of a position of a tooth and/or indicators of a level of movement of a tooth. At block 527, processing logic determines a level of progress associated with the dental treatment plan based on the one or more progress indicators, as discussed in greater detail above. In one embodiment, at least one of a) identifying the one or more progress indicators of treatment progress with respect to the dental treatment plan or b) determining the level of progress associated with the dental treatment plan is performed by processing the patient data using one or more machine learning models that output at least one of the one or more progress indicators or the level of progress associated with the dental treatment plan.

In one example, image data of a patient's dentition (e.g., 3D point clouds and/or 2D images) is input into an ML model, which outputs information on tooth positions and/or orientations. For example, the ML model may perform instance segmentation and/or semantic segmentation, and may output one or more segmentation masks indicating pixels of the input image data representing one or more teeth. In another example, the ML model may perform object recognition and may generate bounding boxes around one or more identified teeth. In embodiments, the ML model may determine tooth numbers and label each identified tooth with an appropriate tooth number. In embodiments, processing logic may compare the determined tooth positions and/or orientations to planned tooth positions and/or orientations for a current treatment stage as provided in the treatment plan. A delta between determined tooth positions/orientations and planned tooth positions/orientations may indicate a level of progress in embodiments. For example, processing logic may determine whether the teeth have moved/rotated less than planned, as planned, or more than planned.

In one example, image data of a patient's dentition is input into an ML model together with information from the dental treatment plan. The information from the dental treatment plan may include, for example, a projection of a 3D model of a planned dentition of the patient for a current stage of treatment onto one or more planes that correspond to plane(s) of the image data, planned tooth positions/orientations for a current treatment stage and/or other information. The ML model may process the received image data of the patient's dentition and the information from the dental treatment plan, and may output information on the one or more progress indicators and/or the level of progress.

In one embodiment, processing logic compares the one or more determined progress indicators to a planned tooth progress indicated in the treatment plan. The level of progress may be determined based on the comparison. In one embodiment, processing logic compares the one or more determined progress indicators to historical progress indicators of historical patient data to determine the level of progress.

At block 528, processing logic may determine whether dynamic treatment is enabled. Dynamic treatment may be an option that can be selected by a doctor. If dynamic treatment is enabled, then processing logic may automatically adjust a treatment plan by lengthening or shortening one or more stages of treatment as appropriate. If dynamic treatment is not enabled, then processing logic may not automatically adjust treatment. If dynamic treatment is not enabled, the method may proceed to block 529, and treatment may proceed as originally planned. If dynamic treatment is enabled, the method may continue to block 530 to determine whether an adjustment to the treatment plan is warranted.

At block 530, processing logic determines one or more actions to perform with respect to the dental treatment plan based at least in part on the determined level of progress. In one embodiment, at block 531 processing logic determines one or more treatment modifications. In one example, at block 532 processing logic advances the patient to a subsequent treatment stage in a series of sequential treatment stages before a preplanned advancement time (i.e., shortens treatment) responsive to determining that the level of progress is further along than planned. In some instances, processing logic may advance to the next treatment stage early if the level of progress is as planned. In one example, at block 534 processing logic retains the patient in a current treatment stage of the series of sequential treatment stages beyond a preplanned advancement time (i.e., lengthens treatment) responsive to determining that the level of progress is less than planned.

In some embodiments, modifying the treatment plan includes changing one or more planned tooth positions/orientations for one or more stages of treatment. For example, if a patient's teeth have not been responding to treatment as planned (e.g., planned tooth movements/rotations are not occurring), then the treatment plan may be modified to change a target final state of the patient's dentition (e.g., by reducing a planned amount of tooth movement/rotation for one or more teeth). This may trigger an adjustment to one or more intermediate treatment stages. In another example, one or more additional treatment stages may be added that have intermediate positions of dentition between two sequential treatment stages of the original treatment plan.

In some instances, the dental treatment plan comprises a combined palatal expansion and orthodontic treatment plan. Such a combined treatment plan may include a series of palatal expansion treatment stages each associated with a distinct palatal expander, a retainer treatment stage associated with a retainer to be worn after palatal expansion and before orthodontic alignment, and a series of orthodontic treatment stages each associated with a distinct orthodontic aligner. If the current stage is palatal expansion treatment stage or a retainer treatment stage, then the treatment modification may be a modification to postpone a transition from a palatal expansion phase of treatment to a retainer phase of treatment and/or an orthodontic alignment stage of treatment. Accordingly, retaining the patient in the current treatment stage beyond the preplanned advancement time may include postponing advancement from palatal expansion treatment to orthodontic treatment. In other instances, processing logic may cause earlier advancement from palatal expansion treatment to orthodontic treatment.

In one embodiment, at block 536 processing logic generates a notification of the determined treatment modification and sends the notification to a patient system (e.g. a computing device of the patient) and/or to a dental professional system (e.g., a computing device of the doctor). The notification may be presented on the patient system and/or dental professional system in a UI of the respective system. The notification to the patient system may include instructions to the patient to wear a subsequent dental appliance corresponding to the subsequent treatment stage or to continue to wear a current dental appliance corresponding to the current treatment stage as appropriate.

In some embodiments, the notification may be sent to the patient system and/or the dental professional system together with or separate from the patient data. A representation of the patient data may be presented in the UI (e.g., GUI) of the patient system and/or dental professional system in embodiments. For example, image data of the patient's dentition, smile, face, etc. may be displayed in the GUI of the patient system and/or dental professional system. In some embodiments, the image data may be annotated with information such as the one or more progress indicators, the determined level of progress, and so on. For example, an overlay providing the progress indicators, the determined level of progress, etc. may be turned on or off based on user input to show or not show such information overlaid with the image data.

The notification may provide one or more visualizations of the number of stages of treatment, the length of treatment, the length of individual stages of treatment, and so on for the treatment plan as originally planned and/or for the treatment plan as modified.

In some embodiments, a comparative visualization of the originally planned length of treatment and the modified length of treatment is output in a GUI of the patient system and/or the dental professional system. Processing logic may determine one or more reasons for the difference between the planned length of treatment and the modified length of treatment in embodiments. For example, processing logic may determine that the patient has had a high compliance (e.g., has been wearing their dental appliances diligently), and that this has contributed to a shorter than planned treatment time. In another example, processing logic may determine that the patient has not been diligent in wearing their dental appliances, and that this has contributed to a longer than planned treatment time. The reasons for the difference between the planned treatment time and the modified treatment time may be output in the GUI. This may help to motivate the patient to start wearing their dental appliances for more time and/or to continue to be diligent in wearing their dental appliances as recommended. In some embodiments, a currently planned completion date and an overall reduction or increase in days of dental treatment may be output to the GUI.

In some embodiments, processing logic generates simulated images of one or more future conditions of the patient's dentition, face, smile, etc. based on a modified treatment plan and/or on the original treatment plan. In some embodiments, current image data of the patient (e.g., of the patient's dentition, face, smile, etc.) is input into a trained ML model along with information from the current or modified treatment plan. The ML model may be a generative model that generates one or more predicted future conditions of the patient's dentition, face, smile, etc. based on the input data. The simulated images may be generated for one or more future time periods associated with one or more intermediate treatment stages and/or a final treatment stage. In some embodiments, simulated images are generated both for the original treatment plan and for the modified treatment plan. The simulated images may be presented to the patient in embodiments. This may enable the patient to visualize what their dentition, face, smile, etc. would look like at a given time period if the original plan was still in place vs. with the modified plan in place.

The machine learning model used to generate the simulated image(s) may be, for example, a generative model such as a GAN. A GAN is a class of artificial intelligence system that uses two artificial neural networks contesting with each other in a zero-sum game framework. The GAN includes a first artificial neural network that generates candidates (e.g., for post-treatment faces of patients) and a second artificial neural network that evaluates the generated candidates. The GAN learns to map from a latent space to a particular data distribution of interest (a data distribution of changes to input images that are indistinguishable from photographs to the human eye), while the discriminative network discriminates between instances from a training dataset and candidates produced by the generator. The generative network's training objective is to increase the error rate of the discriminative network (e.g., to fool the discriminator network by producing novel synthesized instances that appear to have come from the training dataset). The generative network and the discriminator network are co-trained, and the generative network learns to generate images that are increasingly more difficult for the discriminative network to distinguish from real images (from the training dataset) while the discriminative network at the same time learns to be better able to distinguish between synthesized images and images from the training dataset. The two networks of the GAN are trained once they reach equilibrium. The GAN may include a generator network that generates artificial intraoral images and a discriminator network that segments the artificial intraoral images. In embodiments, the discriminator network may be a MobileNet.

In one embodiment, one or more machine learning model is a conditional generative adversarial (cGAN) network, such as pix2pix. These networks not only learn the mapping from input image to output image, but also learn a loss function to train this mapping. GANs are generative models that learn a mapping from random noise vector z to output image y, G: z→y. In contrast, conditional GANs learn a mapping from observed image x and random noise vector z, to y, G: {x, z}→y. The generator G is trained to produce outputs that cannot be distinguished from “real” images by an adversarially trained discriminator, D, which is trained to do as well as possible at detecting the generator's “fakes”. The generator may include a U-net or encoder-decoder architecture in embodiments. The discriminator may include a MobileNet architecture in embodiments. An example of a cGAN machine learning architecture that may be used is the pix2pix architecture described in Isola, Phillip, et al. “Image-to-image translation with conditional adversarial networks.” arXiv preprint (2017).

In some embodiments, multiple simulated images are generated for different future states of dental treatment. A first set of simulated images may be generated based on the original treatment plan, and an additional set of images may be generated for a modified treatment plan. Other sets of simulated images may also be generated for other alternative modified treatment plans in embodiments. One or more of the simulated images may be shown to a patient and/or doctor so that they can assess what the patient's dentition, face and/or smile would like at one or more future points in time if the original treatment plan, the modified treatment plan or an alternative modified treatment plan were implemented. More information about simulating images of patients for treatment plans can be found in U.S. Pat. Nos. 9,642,678, 11,147,652, 11,020,205, 11,759,291, 12,133,783, 10,835,349, 11,801,121, 11,151,753, and U.S. Pat. No. 11,723,748, which are incorporated herein by reference in their entirety.

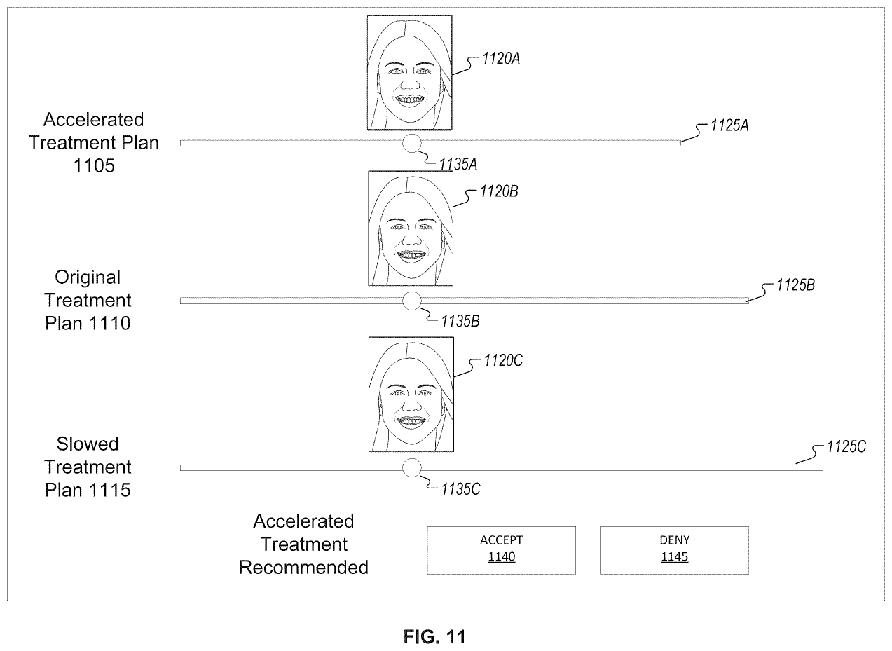

FIG. 11 illustrates a view of multiple treatment timelines in a graphical user interface (GUI) for a patient system or dental professional device. As shown, the GUI may show a comparison of one or more treatment plans, such as an accelerated treatment plan 1105, an original treatment plan 1110, and a slowed treatment plan 1115. The accelerated treatment plan 1105 may be a modified treatment plan that may be implemented if certain treatment acceleration criteria are satisfied (e.g., patient is compliant in wearing dental appliances, treatment is progressing on track or faster than anticipated, etc.). The slowed treatment plan 1115 may be a modified treatment plan that may be implemented if certain slowing criteria are satisfied (e.g., patient is not compliant in wearing dental appliances, patient's teeth are responding to treatment plan slower than anticipated, etc.). In some embodiments, only two treatment plans are shown, including the original treatment plan 1110 and a recommended modified treatment plan. The recommended modified treatment plan may be an acerbated treatment plan or a slowed treatment plan, depending on how the patient's dentition is responding to the original treatment plan. For example, the original treatment plan may call for a dental appliance change every 8 days, and the modified treatment plan may be an accelerated treatment plan that calls for a dental appliance change every 6 days.

In one embodiment, a treatment timeline 1125A, 1125B, 1125C is shown for each of the treatment plans 1105-1115. The different treatment timelines 1125A-C may have different lengths due to different treatment times of the different treatment plans 1105-1115. For example, accelerated treatment plan 1105 has a shortest treatment timeline 1125A, original treatment plan 1110 has a longer treatment timeline 1125B, and slowed treatment plan 1115 has a longest treatment timeline 1125C. Each of the treatment timelines 1125A-C may be a slider that has a slider knob 1135A-C. A user may drag any of the slider knobs 1135A-C along the respective slider to select a future date, a current date, or a past date. Over each of the slider knobs 1135A-C is a thumbnail image 1120A-C of the patient's face, dentition and/or smile. The thumbnail image 1120A of accelerated treatment plan 1105 is an image showing what the patient's face, smile and/or dentition will or did look like at a particular future or past date based on execution of the accelerated treatment plan 1105 moving forward. The thumbnail image 1120B of original treatment plan 1110 is an image showing what the patient's face, smile and/or dentition will or did look like at a particular future or past date based on execution of the original treatment plan 1110 moving forward. The thumbnail image 1120C of slowed treatment plan 1115 is an image showing what the patient's face, smile and/or dentition will or did look like at a particular future or past date based on execution of the slowed treatment plan 1115 moving forward.

If a past date is selected, an image of the patient's face, smile and/or dentition at the prior date may be presented. The past date images for each of the treatment plans may be the same in embodiments. If a future date is selected, a simulated image of the patient's face, smile and/or dentition at the selected future date is presented. Adjusting any of the slider knobs 1135A-C may cause all of the slider knobs 1135A-C to be adjusted by a same amount. Accordingly, a patient and/or doctor may view and compare thumbnail images of what the patient's face, dentition and/or smile will look like at any given future time. A user may select a thumbnail image 1120A-C associated with a treatment plan 1105A-C to cause enlarged versions of the selected thumbnail image for the treatment plan and/or the corresponding thumbnail images of the other treatment plans to be shown.

In an example, a patient may have an important event upcoming (e.g., such as a wedding), and may want to see what their smile will look like at the event. The patient may drag the slider knob to the date of the event and compare what their dentition will look like at that date under the different treatment plans. This may provide incentive for the patient to comply with recommended wear times of dental appliances to enable them to qualify for the accelerated treatment plan 1105.

In some embodiments, processing logic recommends modifying the treatment plan (e.g., accelerating or slowing down the treatment plan), but does not implement such changes automatically. For example, processing logic may output a recommendation to implement the modified treatment plan (e.g., the accelerated treatment plan) as shown in the GUI of FIG. 11. The patient and/or doctor may be presented with options (e.g., via buttons) to accept 1140 or deny 1145 the recommended modification to the treatment plan. They may accept or deny the recommended modification after viewing the comparison of the different treatment plan lengths, projected outcomes at different times, and so on in some embodiments.

In some embodiments, a video is generated that shows the progression of the patient's dentition, smile, face, etc. from pre-treatment to post-treatment for the original treatment plan and/or for the modified treatment plan. The video may be output to the GUI of the user system and/or the dental professional system in embodiments.

Returning to FIG. 5B, in some embodiments, the dental treatment plan includes an assessment schedule that indicates one or more time points (e.g., predefined time intervals) in the treatment plan at which patient data is to be gathered (e.g., points at which the patient is instructed to take one or more images of their dentition with and/or without a current dental appliance being worn). At block 538, processing logic may adjust the assessment schedule by shortening or lengthening one or more of the predefined time intervals. For example processing logic may increase or decrease the frequency level at which patient data is generated/received based on the determined level of progress. In an example, if the patient's dentition has repeatedly been progressing as planned according to the dental treatment plan, then a frequency of collecting patient data may be decreased. However, if the patient's dentition is not progressing as planned according to the dental treatment plan, then the frequency may be increased.

As discussed, the dental treatment plan may be divided into multiple treatment stages, each of which may be associated with a unique dental appliance (e.g., a palatal expander, retainer, orthodontic aligner, etc.) that is customized for a particular patient and for a particular stage of treatment for that patient. A particular dental appliance may not fit a patient's dentition at a particular stage of treatment if the patient's dentition did not respond to treatment as originally planned. For example, an intermediate orthodontic treatment stage may include an orthodontic aligner that will fit a patient's dentition that have moved by a planned amount. However, if one or more teeth of the patient have not moved by the planned amount then the orthodontic aligner would not fit onto that patient's dental arch.

In some embodiments, rather than manufacturing all of the dental appliances associated with a dental treatment plan at once (e.g., at a start of treatment), the dental appliances of the dental treatment plan are manufactured in batches. Accordingly, manufacturing of a batch of dental appliances associated with one or more later stages of treatment may be delayed until the treatment plan calls for those dental appliances to be worn by the patient. If the treatment plan has been modified (e.g., by changing a final target dentition and/or a target dentition or one or more stages of treatment), then the shape/design of the dental appliances for those treatment stages may be different than originally planned. By delaying the manufacture of the dental appliances for those one or more treatment stages, scrapping and remanufacturing of dental appliances for those treatment stages may be reduced or eliminated.

In some embodiments, at block 539 processing logic determines whether to order a next set or batch of dental appliances. For example, processing logic may predict that a patient will be finished with a previously manufactured batch of dental appliances at a particular future date (e.g., based on whether treatment has been accelerated, lengthened, on track, etc. for one or more prior and/or current treatment stages). Based on the predicted time at which the patient will complete use of the already manufactured batch of dental appliances, processing logic may select an appropriate time to order a next batch of dental appliances to ensure that the next batch of dental appliances will be manufacture and shipped to the patient before the treatment plan calls for the patient to wear those dental appliances.

FIG. 5C illustrates a flow diagram of an example method 540 for modifying an orthodontic treatment plan, in accordance with some embodiments of the present disclosure. At block 542, processing logic receives patient data associated with one or more stages of a dental treatment plan. The patient data may include any of the types of patient data discussed herein, such as 2D images, 3D point clouds, etc. of a patient's dentition, face, smile, etc. Other patient data may include, for example, data of a dentition of the patient, biomarker data indicative of changes in the dentition, pressure data indicative of a level of pressure exerted by the dentition on a dental appliance, data indicative of an electrical parameter associated with a position of a tooth with respect to a dental appliance, and so on. The patient data may be generated without a need for the patient to visit a healthcare provider in embodiments. Processing logic may also access and/or receive data of the dental treatment plan in embodiments.

At block 526, processing logic processes the patient data to identify one or more conditions of the patient's dentition, to make one more determinations or characterizations about the patient's dentition, and so on. In one embodiment, processing logic processes the patient data to determine a) one or more progress indicators of treatment progress with respect to the dental treatment plan, b) a level of progress associated with the dental treatment plan, c) oral health condition(s) of patient, d) any missing dental auxiliaries, e) a level of wear of a dental appliance, and/or f) one or more relapsed teeth. In one embodiment, the one or more observations/determinations/characterizations, etc. are made at least in part by inputting the patient data into one or more trained machine learning models. The trained machine learning model(s) may process the patient data and/or other input data such as data from the dental treatment plan (e.g., a current stage of treatment, number of stages of treatment, a planned timing of a next stage of treatment, image(s) of a planned current state of the patient's dentition, etc.) to generate an output. The output may be, observations/determinations/characterizations that may include, for example, a) one or more progress indicators of treatment progress with respect to the dental treatment plan, b) a level of progress associated with the dental treatment plan, c) one or more identified oral health condition(s) of the patient, d) identification of any missing dental auxiliaries, e) identification of a level of wear of a dental appliance, f) identification of one or more relapsed teeth, and so on. For example, as discussed above, many different types of progress indicators may be identified, such one or more tooth positions, one or more tooth orientations, arch width, and so on. The one or more progress indicators comprises, for example, indicators of a position of a tooth and/or indicators of a level of movement of a tooth.

One or more ML models may perform instance segmentation and/or semantic segmentation, and may output one or more segmentation masks indicating pixels of the input image data representing one or more teeth, regions at which one or more oral health conditions are identified (e.g., a caries, an area of gingival inflammation, a chipped tooth, a crack in a tooth, etc.), regions at which one or more expected dental auxiliaries are missing, areas of wear on a dental appliance, identification of one or more relapsed teeth, etc. One or more ML models may perform object recognition and may generate bounding boxes around one or more identified teeth, regions of oral health conditions, missing dental auxiliaries, relapsed teeth, etc. In embodiments, a first ML model may perform segmentation of the image data, and may assign labels to segmented objects such as tooth numbers to teeth. In embodiments, the image data and/or the segmentation information of the image data may be processed by one or more further ML models to determine and output a) one or more progress indicators of treatment progress with respect to the dental treatment plan, b) a level of progress associated with the dental treatment plan, c) one or more identified oral health condition(s) of the patient, d) identification of any missing dental auxiliaries, e) identification of a level of wear of a dental appliance, f) identification of one or more relapsed teeth, and so on.

At block 546, processing logic determines one or more actions to perform based on the observations made at block 544 (e.g., based on at least one of a) one or more progress indicators of treatment progress with respect to the dental treatment plan, b) a level of progress associated with the dental treatment plan, c) oral health condition(s) of patient, d) any missing dental auxiliaries, e) a level of wear of a dental appliance, and/or f) one or more relapsed teeth). Processing logic may then perform the one or more determined actions.

In some embodiments, the determined actions include outputting a notice of the observations (e.g., oral health conditions, missing dental auxiliaries, worn aligner, etc.) to the patient system and/or to the dental professional system. Additionally, the determined actions may include, at block 554, recommending and/or scheduling an in-person patient visit with the doctor in view of the determined observations.

In some embodiments, the determined actions include, at block 548, recommending and/or performing one or more modifications to the dental treatment plan. The modifications may include any of the aforementioned treatment plan modifications. In one example, processing logic advances the patient to a subsequent treatment stage in a series of sequential treatment stages before a replanned advancement time (i.e., shortens treatment) responsive to determining that the level of progress is further along than planned and/or that there are no oral health conditions. In some instances, processing logic may advance to the next treatment stage early if the level of progress is as planned and/or there are no oral health conditions identified. In one example, processing logic retains the patient in a current treatment stage of the series of sequential treatment stages beyond a preplanned advancement time (i.e., lengthens treatment) responsive to determining that the level of progress is less than planned and/or that one or more oral health conditions are identified.

In some embodiments, modifying the treatment plan includes changing one or more planned tooth positions/orientations for one or more stages of treatment. For example, if a patient's teeth have not been responding to treatment as planned (e.g., planned tooth movements/rotations are not occurring), then the treatment plan may be modified to change a target final state of the patient's dentition (e.g., by reducing a planned amount of tooth movement/rotation for one or more teeth).

In one example, one or more oral health conditions are identified at block 544, and the one or more actions are determined and/or performed based at least in part on the one or more oral health conditions. In one embodiment, oral health conditions are identified as set forth in U.S. Pat. No. 12,127,814, issued Oct. 29, 2024, which is incorporated by reference herein in its entirety.

Examples of such oral health conditions include gingival inflammation, a tooth crack, a missing tooth, a chipped tooth, and caries. In an example, processing logic may determine one or more treatment modifications to the dental treatment plan in view of the identified oral health condition(s). In one embodiment, processing logic outputs a recommendation to pause execution of the dental treatment plan while the one or more oral health conditions are addressed. In one embodiment, processing logic retains the patient in a current treatment stage of the series of sequential treatment stages beyond a preplanned advancement time in view of the identified oral health condition(s). By retaining the patient in the current stage of treatment longer than planned, this may enable the oral health condition(s) to be addressed and/or to heal while dental treatment is slowed down or paused. For example, an identified oral health condition may be gingival inflammation, which may be caused or exacerbated by orthodontic work. Accordingly, a patient may be retained in a current stage of orthodontic treatment for a lengthened period of time to give time for the patient's mouth to adapt to the current tooth positions before advancing to a next treatment stage.

In one embodiment, at block 550 processing logic determines one or more oral healthcare activities likely to ameliorate the one or more oral health conditions. For example, gingival inflammation may be ameliorated by a proper dental healthcare routine of brushing, flossing, rinsing, and so on. At block 552, processing logic may then output a recommendation for the patient to adjust an oral healthcare routine to include the one or more oral healthcare activities to address the one or more oral health conditions. Such a recommendation may be output in a GUI of a patient system in embodiments.

In some embodiments, processing logic determines a severity level of the one or more identified oral health conditions. In some embodiments, the ML model that identifies oral health conditions also estimates a severity level of those oral health conditions and outputs the estimated severity level information. Alternatively, or additionally, further processing (e.g., image processing) may be performed to measure one or more properties of the identified oral health conditions. For example, processing logic may measure a width of a caries, a length of a tooth crack, an level of gingival inflammation (e.g., based on comparing a volume or surface area difference between swollen gums as captured and planned or previously captured patient gums). One or more determined/performed actions may be based at least in part on the severity level of the oral health condition(s) in embodiments.

Some oral health conditions warrant an in-person patient visit with a doctor. For example, an extensive tooth crack or a serious caries should be addressed by a doctor. Accordingly, in some instances, processing logic outputs a recommendation for a patient to schedule an in-person visit with their doctor and/or schedules such a doctor visit. Alternatively, or additionally, such a recommendation may be output to the dental professional system to notify the doctor of the oral health conditions. In an example, processing logic outputs a notice of the one or more oral health conditions and/or their severity levels to a device of a doctor. The doctor may provide instructions associated with the one or more oral health conditions, which may be received by processing logic. Processing logic may then send the doctor instructions to a device of the patient. The instructions may include, for example, instructions for the patient to perform an in-person visit with the doctor, which processing logic may facilitate by scheduling in some embodiments.

In some embodiments, a doctor provides doctor preferences with regards to which types of oral health conditions and/or what severity levels of oral health conditions warrant an in-person patient visit. If such information is available, processing logic may determine whether identified oral health conditions and/or severity levels satisfy one or more in-patient visit criteria as specified by the doctor. If so, a recommendation for the patient to visit the doctor may be sent to the doctor and/or patient, and/or an in-person visit may be automatically scheduled.

In some instances it may be beneficial for dental treatment to be paused while treatment of one or more oral health conditions is performed. In other instances, it may be optimal to perform restorative dental treatment to address one or more oral health conditions in parallel to continued palatal expansion and/or orthodontic dental treatment. Accordingly, processing logic may determine whether to recommend pausing dental treatment and/or performing restorative dental treatment to address identified oral health conditions. In embodiments, such recommendations may be output by one or more of the aforementioned ML models. In some embodiments, a recommendation to perform restorative treatment may be output to the doctor and/or patient devices at block 556.

In some instances, the dental treatment plan calls for dental auxiliaries on one or more teeth of the patient. One type of dental auxiliary, referred to as dental attachments, may facilitate application of one or more forces to one or more teeth, such as application of rotational forces to teeth that may be difficult to achieve without such attachments. Other types of dental auxiliaries perform other functions that may have clinical significance. Accordingly, it can be important that the dental auxiliaries are applied to the patient's teeth as planned. In one embodiment, at block 558 processing logic determines that the patient has one or more missing dental auxiliaries. A missing dental auxiliary may be identified by a trained ML model in some instances, as set forth above. In some embodiments, missing dental auxiliaries are identified by comparing patient data (e.g., an image of a patient's current dentition) to data from the dental treatment plan (e.g., a 3D model or point cloud of the planned dentition of the patient for the current stage of treatment or a projection of the 3D model or point cloud onto a plane of one or more images of the image data). Based on the comparison, processing logic may identify regions where dental auxiliaries should be but are absent. In some embodiments, processing logic may additionally make a recommendation to change one or more dental auxiliaries if one or more dental auxiliary replacement criteria are satisfied. For example, if the patient has been compliant in wearing their dental appliances but a difficult tooth movement is not proceeding as planned, this may be because a dental attachment was not positioned at an optimal location, that a larger dental attachment may be needed and/or that a dental attachment having a different shape may be needed. Accordingly, if the patient has been compliant and an achieved tooth movement or rotation is behind schedule, processing logic may identify these facts and recommend replacing a current dental attachment with a modified dental attachment. In some cases, processing logic may identify a damaged dental auxiliary (e.g., an ML model may process an image of a patient's dentition, which may output an indication of a damaged dental auxiliary). In such instances, processing logic may recommend replacing the dental auxiliary that has been damaged.

Typically, dental auxiliaries to a patient's teeth are applied at a start of treatment. However, those dental auxiliaries may not be needed for all stages of treatment. For example, one or more stages of treatment may use the dental auxiliaries to apply specific forces to one or more teeth. After those stages of treatment are complete, then the dental auxiliaries may no longer have a clinical significance. Accordingly, in some embodiments processing logic determines whether any identified missing dental auxiliaries have clinical significance. Such a determination may be made by determining whether tooth rotations/motions facilitated by the missing dental auxiliaries have already been performed or are yet to be performed. The missing dental auxiliary(ies) may have clinical significance if one or more tooth movements achieved using forces facilitated by the missing auxiliary(ies) have not yet been performed. If there are no future movements/rotations of teeth that require the missing dental auxiliaries for the remainder of treatment, then the missing dental auxiliaries may not have clinical significance.

Processing logic may then perform one or more actions based on the identified missing dental auxiliaries and/or based on the determination of whether the missing dental auxiliaries have clinical significance. For example, if the missing dental auxiliaries have clinical significance, then a recommendation for the patient to have an in-person visit with the doctor to enable the doctor to reattach the missing dental auxiliaries may be output to the patient device and/or the doctor device. If the missing dental auxiliaries are determined not to have clinical significance, then no such recommendation may be output. In one embodiment, if a missing dental auxiliary with clinical significance is identified, then treatment may be slowed down (e.g., the timing of advancing to a next treatment stage may be delayed). For example, a current treatment stage may be prolonged until the patient has a next scheduled in-patient doctor visit. In some embodiments, a notice of a missing dental auxiliary and/or whether it has clinical significance may be output to a device of a doctor, enabling the doctor to make a decision on how to proceed.

One or more images of the patient's dentition included in the patient data may be images taken while the patient was wearing their current dental appliance. Some types of dental appliances such as retainers are meant to be worn for a long period of time, such as for months. During that time, the dental appliance can become worn (e.g., due to a patient biting, chewing, clenching, etc. their teeth while wearing the dental appliance). Excessive wear on a dental appliance may reduce the effectiveness of the dental appliance for its intended purpose (e.g., may reduce an ability of a retainer to retain the patient's teeth in their current positions). Accordingly, in one embodiment at block 544 a level of wear of the patient's dental appliance is determined based on processing of the image of the patient's dentition in which the patient is wearing the dental appliance using an ML model, which outputs an estimated amount of wear on the dental appliance. Processing logic may compare the determined level of wear to a wear threshold. If the level of wear exceeds the wear threshold, then a determination may be made that the dental appliance should be replaced. Accordingly, processing logic may automatically order a new dental appliance (e.g., a new retainer) responsive to determining that the level of wear exceeds the wear threshold.

In some cases a patient's teeth may relapse from an adjusted position towards an original position after orthodontic alignment. This may cause a retainer that was designed to be worn by the patient after completion of orthodontic treatment to no longer fit the patient's dental arch. Accordingly, in one embodiment processing logic determines that one or more teeth of the patient have relapsed and designs a new retainer in view of the one or more teeth of the patient that have relapsed. The new retainer may be a retainer that will fit on the patient's current dental arch and that will mitigate further regression of the patient's teeth. Processing logic may then order the new retainer. In some instances, it may be beneficial for the patient to do an in-person visit with their doctor to enable the doctor to perform an intraoral scan of the patient's current dentition. A 3D model of the patient's current dentition may be generated from the intraoral scan. The new retainer may then be generated based on the 3D model of the patient's current dentition.

In some instances, if the patient's teeth have relapsed, then an orthodontic treatment plan may be adjusted to cause the patient to have one or more additional treatment stages to reposition the patient's teeth from their relapsed state back to the planned final state of the teeth. One or more new orthodontic aligners may then be generated for the additional treatment stages.

As discussed above, in some embodiments processing logic is able to assess an overall difficulty/challenge level of an orthodontic treatment and/or of one or more individual stages of the orthodontic treatment. In one embodiment, processing logic determines whether a next stage of orthodontic treatment has a difficulty/challenge level that is above a difficulty threshold. Such stages may be categorized difficult stages (e.g., as stages comprising challenging tooth movements). If the next treatment stage is categorized as a difficult stage, then processing logic may output one or more notice to the patient in anticipation of the challenging stage. The notice may include, for example, a notice for the patient to perform at least one of the following: a) be diligent in wearing the dental appliance for the next treatment stage; or b) chew on one or more chewable objects using the one or more teeth after seating the dental appliance on a dental arch of the patient to secure the dental appliance to the one or more teeth. If the patient follows the recommendations of the notice, this may improve a probability that the planned tooth movements associated with the difficult treatment stage will be successful.

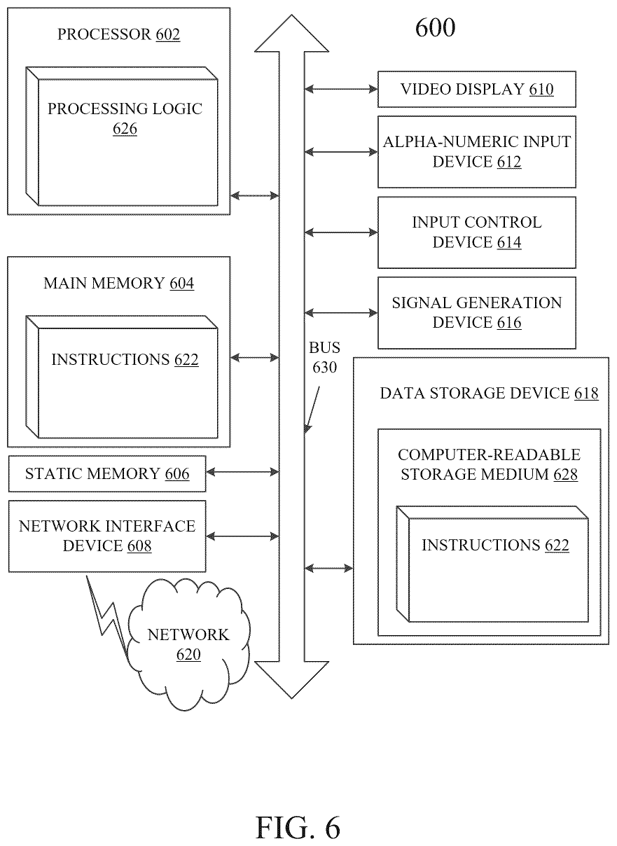

FIG. 6 illustrates a block diagram of an example processing device 600 operating in accordance with one or more aspects of the present disclosure. In one implementation, the processing device 600 may be a part of any computing device of FIG. 2A, or any combination thereof. Example processing device 600 may be connected to other processing devices in a LAN, an intranet, an extranet, and/or the Internet. The processing device 600 may be a personal computer (PC), a set-top box (STB), a server, a network router, switch or bridge, or any device capable of executing a set of instructions (sequential or otherwise) that specify actions to be taken by that device. Further, while only a single example processing device is illustrated, the term “processing device” shall also be taken to include any collection of processing devices (e.g., computers) that individually or jointly execute a set (or multiple sets) of instructions to perform any one or more of the methods discussed herein.

Example processing device 600 may include a processor 602 (e.g., a CPU), a main memory 604 (e.g., read-only memory (ROM), flash memory, dynamic random access memory (DRAM) such as synchronous DRAM (SDRAM), etc.), a static memory 606 (e.g., flash memory, static random access memory (SRAM), etc.), and a secondary memory (e.g., a data storage device 618), which may communicate with each other via a bus 630.

Processor 602 represents one or more general-purpose processing devices such as a microprocessor, central processing unit, or the like. More particularly, processor 602 may be a complex instruction set computing (CISC) microprocessor, reduced instruction set computing (RISC) microprocessor, very long instruction word (VLIW) microprocessor, processor implementing other instruction sets, or processors implementing a combination of instruction sets. Processor 602 may also be one or more special-purpose processing devices such as an application specific integrated circuit (ASIC), a field programmable gate array (FPGA), a digital signal processor (DSP), network processor, or the like. In accordance with one or more aspects of the present disclosure, processor 602 may be configured to execute instructions (e.g. processing logic 626 may implement the optimization module of FIG. 2A).

Example processing device 600 may further include a network interface device 608, which may be communicatively coupled to a network 620. Example processing device 600 may further comprise a video display 610 (e.g., a liquid crystal display (LCD), a touch screen, or a cathode ray tube (CRT)), an alphanumeric input device 612 (e.g., a keyboard), an input control device 614 (e.g., a cursor control device, a touch-screen control device, a mouse), and a signal generation device 616 (e.g., an acoustic speaker).

Data storage device 618 may include a computer-readable storage medium (or, more specifically, a non-transitory computer-readable storage medium) 628 on which is stored one or more sets of executable instructions 622. In accordance with one or more aspects of the present disclosure, executable instructions 622 may comprise executable instructions (e.g. instructions for implementing the optimization module of FIG. 2A).

Executable instructions 622 may also reside, completely or at least partially, within main memory 604 and/or within processor 602 during execution thereof by example processing device 600, main memory 604 and processor 602 also constituting computer-readable storage media. Executable instructions 622 may further be transmitted or received over a network via network interface device 608.

While the computer-readable storage medium 628 is shown in FIG. 6 as a single medium, the term “computer-readable storage medium” should be taken to include a single medium or multiple media (e.g., a centralized or distributed database, and/or associated caches and servers) that store the one or more sets of operating instructions. The term “computer-readable storage medium” shall also be taken to include any medium that is capable of storing or encoding a set of instructions for execution by the machine that cause the machine to perform any one or more of the methods described herein. The term “computer-readable storage medium” shall accordingly be taken to include, but not be limited to, solid-state memories, and optical and magnetic media.

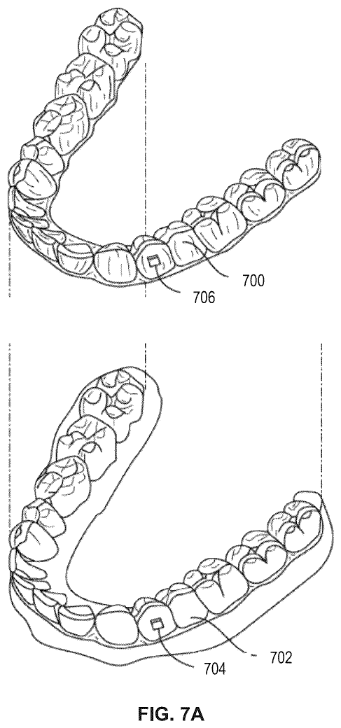

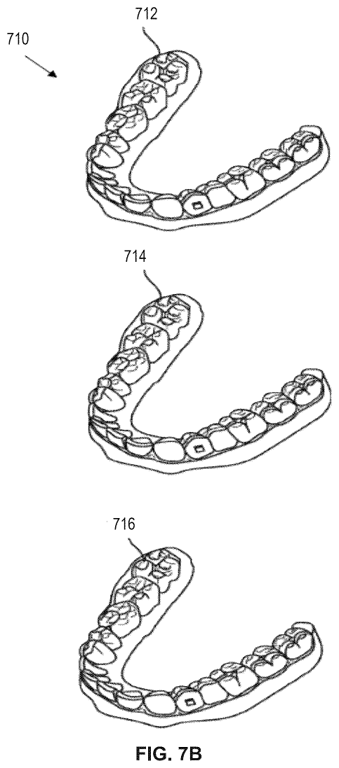

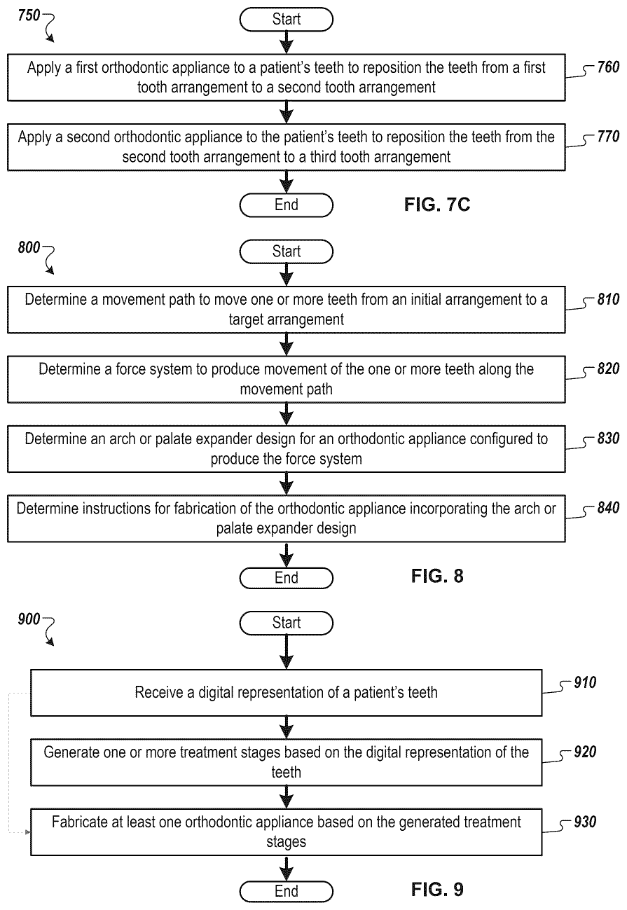

FIG. 7A illustrates an exemplary tooth repositioning dental appliance or aligner 700 that can be worn by a patient in order to achieve an incremental repositioning of individual teeth 702 in the jaw. The aligner 700 may be formed using modular trays and/or global plan data, as disclosed herein. The appliance can include a shell (e.g., a continuous polymeric shell or a segmented shell) having teeth-receiving cavities that receive and resiliently reposition the teeth. An appliance or portion(s) thereof may be indirectly fabricated using a physical model of teeth. For example, an appliance (e.g., polymeric appliance) can be formed using a physical model of teeth and a sheet of suitable layers of polymeric material. A “polymeric material,” as used herein, may include any material formed from a polymer. A “polymer,” as used herein, may refer to a molecule composed of repeating structural units connected by covalent chemical bonds often characterized by a substantial number of repeating units (e.g., equal to or greater than 3 repeating units, optionally, in some embodiments equal to or greater than 10 repeating units, in some embodiments greater or equal to 30 repeating units) and a high molecular weight (e.g. greater than or equal to 10,000 Da, in some embodiments greater than or equal to 50,000 Da or greater than or equal to 100,000 Da).

Although polymeric aligners are discussed herein, the techniques disclosed may also be applied to aligners having different materials. Some embodiments are discussed herein with reference to orthodontic aligners (also referred to simply as aligners). However, embodiments also extend to other types of shells formed over molds, such as orthodontic retainers, orthodontic splints, sleep appliances for mouth insertion (e.g., for minimizing snoring, sleep apnea, etc.), palatal expanders and/or shells for non-dental applications. Accordingly, it should be understood that embodiments herein that refer to aligners also apply to other types of shells.

The aligner 700 can fit over all teeth present in an upper or lower jaw, or less than all of the teeth. The appliance can be designed specifically to accommodate the teeth of the patient (e.g., the topography of the tooth-receiving cavities matches the topography of the patient's teeth), and may be fabricated based on positive or negative models of the patient's teeth generated by impression, scanning, and the like. Alternatively, the appliance can be a generic appliance configured to receive the teeth, but not necessarily shaped to match the topography of the patient's teeth. In some cases, only certain teeth received by an appliance will be repositioned by the appliance while other teeth can provide a base or anchor region for holding the appliance in place as it applies force against the tooth or teeth targeted for repositioning. In some cases, some, most, or even all of the teeth will be repositioned at some point during treatment. Teeth that are moved can also serve as a base or anchor for holding the appliance as it is worn by the patient. Typically, no wires or other means will be provided for holding an appliance in place over the teeth. In some cases, however, it may be desirable or necessary to provide individual dental auxiliaries (e.g., dental attachments or other anchoring elements) 704 on teeth 702 with corresponding receptacles or apertures 706 in the aligner 700 so that the appliance can apply a selected force on the tooth. Exemplary appliances, including those utilized in the Invisalign® System, are described in numerous patents and patent applications assigned to Align Technology, Inc. including, for example, in U.S. Pat. Nos. 6,450,807, and 5,975,893, as well as on the company's website, which is accessible on the World Wide Web (see, e.g., the URL “invisalign.com”). Examples of tooth-mounted dental auxiliaries suitable for use with orthodontic appliances are also described in patents and patent applications assigned to Align Technology, Inc., including, for example, U.S. Pat. Nos. 6,309,215 and 6,830,450.

FIG. 7B illustrates a tooth repositioning system 710 including a plurality of appliances 712, 714, 716. Any of the appliances described herein can be designed and/or provided as part of a set of a plurality of appliances used in a tooth repositioning system. Each appliance may be configured so a tooth-receiving cavity has a geometry corresponding to an intermediate or final tooth arrangement intended for the appliance. The patient's teeth can be progressively repositioned from an initial tooth arrangement to a target tooth arrangement by placing a series of incremental position adjustment appliances over the patient's teeth. For example, the tooth repositioning system 710 can include a first appliance 712 corresponding to an initial tooth arrangement, one or more intermediate appliances 714 corresponding to one or more intermediate arrangements, and a final appliance 716 corresponding to a target arrangement. A target tooth arrangement can be a planned final tooth arrangement selected for the patient's teeth at the end of all planned orthodontic treatment. Alternatively, a target arrangement can be one of some intermediate arrangements for the patient's teeth during the course of orthodontic treatment, which may include various different treatment scenarios, including, but not limited to, instances where surgery is recommended, where interproximal reduction (IPR) is appropriate, where a progress check is scheduled, where anchor placement is best, where palatal expansion is desirable, where restorative dentistry is involved (e.g., inlays, onlays, crowns, bridges, implants, veneers, and the like), etc. As such, it is understood that a target tooth arrangement can be any planned resulting arrangement for the patient's teeth that follows one or more incremental repositioning stages. Likewise, an initial tooth arrangement can be any initial arrangement for the patient's teeth that is followed by one or more incremental repositioning stages.

In some embodiments, the appliances 712, 714, 716 (or portions thereof) can be produced using indirect fabrication techniques, such as by thermoforming over a positive or negative mold. Indirect fabrication of an orthodontic appliance can involve producing a positive or negative mold of the patient's dentition in a target arrangement (e.g., by rapid prototyping, milling, etc.) and thermoforming one or more sheets of material over the mold in order to generate an appliance shell.

In an example of indirect fabrication, a mold of a patient's dental arch may be fabricated from a digital model of the dental arch, and a shell may be formed over the mold (e.g., by thermoforming a polymeric sheet over the mold of the dental arch and then trimming the thermoformed polymeric sheet). The fabrication of the mold may be performed by a rapid prototyping machine (e.g., a stereolithography (SLA) 3D printer). The rapid prototyping machine may receive digital models of molds of dental arches and/or digital models of the appliances 712, 714, 716 after the digital models of the appliances 712, 714, 716 have been processed by processing logic of a computing device, such as the computing device in FIG. 6. The processing logic may include hardware (e.g., circuitry, dedicated logic, programmable logic, microcode, etc.), software (e.g., instructions executed by a processing device), firmware, or a combination thereof.

To manufacture the molds, a shape of a dental arch for a patient at a treatment stage is determined based on a treatment plan. In the example of orthodontics, the treatment plan may be generated based on an intraoral scan of a dental arch to be modeled. The intraoral scan of the patient's dental arch may be performed to generate a 3D virtual model of the patient's dental arch (mold). For example, a full scan of the mandibular and/or maxillary arches of a patient may be performed to generate 3D virtual models thereof. The intraoral scan may be performed by creating multiple overlapping intraoral images from different scanning stations and then stitching together the intraoral images to provide a composite 3D virtual model. In other applications, virtual 3D models may also be generated based on scans of an object to be modeled or based on use of computer aided drafting techniques (e.g., to design the virtual 3D mold). Alternatively, an initial negative mold may be generated from an actual object to be modeled (e.g., a dental impression or the like). The negative mold may then be scanned to determine a shape of a positive mold that will be produced.

Once the virtual 3D model of the patient's dental arch is generated, a dental practitioner and/or treatment planning software may determine a desired treatment outcome, which includes final positions and orientations for the patient's teeth. Processing logic may then determine a number of treatment stages to cause the teeth to progress from starting positions and orientations to the target final positions and orientations. The shape of the final virtual 3D model and each intermediate virtual 3D model may be determined by computing the progression of tooth movement throughout orthodontic treatment from initial tooth placement and orientation to final corrected tooth placement and orientation. For each treatment stage, a separate virtual 3D model of the patient's dental arch at that treatment stage may be generated. The shape of each virtual 3D model will be different. The original virtual 3D model, the final virtual 3D model and each intermediate virtual 3D model is unique and customized to the patient.

Accordingly, multiple different virtual 3D models (digital designs) of a dental arch may be generated for a single patient. A first virtual 3D model may be a unique model of a patient's dental arch and/or teeth as they presently exist, and a final virtual 3D model may be a model of the patient's dental arch and/or teeth after correction of one or more teeth and/or a jaw. Multiple intermediate virtual 3D models may be modeled, each of which may be incrementally different from previous virtual 3D models.

Each virtual 3D model of a patient's dental arch may be used to generate a unique customized physical mold of the dental arch at a particular stage of treatment. The shape of the mold may be at least in part based on the shape of the virtual 3D model for that treatment stage. The virtual 3D model may be represented in a file such as a computer aided drafting (CAD) file or a 3D printable file such as a stereolithography (STL) file. The virtual 3D model for the mold may be sent to a third party (e.g., clinician office, laboratory, manufacturing facility or other entity). The virtual 3D model may include instructions that will control a fabrication system or device in order to produce the mold with specified geometries.

A clinician office, laboratory, manufacturing facility or other entity may receive the virtual 3D model of the mold, the digital model having been created as set forth above. The entity may input the digital model into a rapid prototyping machine. The rapid prototyping machine then manufactures the mold using the digital model. One example of a rapid prototyping manufacturing machine is a 3D printer. 3D printing includes any layer-based additive manufacturing processes. 3D printing may be achieved using an additive process, where successive layers of material are formed in proscribed shapes. 3D printing may be performed using extrusion deposition, granular materials binding, lamination, photopolymerization, continuous liquid interface production (CLIP), or other techniques. 3D printing may also be achieved using a subtractive process, such as milling.

Appliances may be formed from each mold and when applied to the teeth of the patient, may provide forces to move the patient's teeth as dictated by the treatment plan. The shape of each appliance is unique and customized for a particular patient and a particular treatment stage. In an example, the appliances 712, 714, 716 can be pressure formed or thermoformed over the molds. Each mold may be used to fabricate an appliance that will apply forces to the patient's teeth at a particular stage of the orthodontic treatment. The appliances 712, 714, 716 each have teeth-receiving cavities that receive and resiliently reposition the teeth in accordance with a particular treatment stage.

In one embodiment, a sheet of material is pressure formed or thermoformed over the mold. The sheet may be, for example, a sheet of polymeric (e.g., an elastic thermopolymeric, a sheet of polymeric material, etc.). To thermoform the shell over the mold, the sheet of material may be heated to a temperature at which the sheet becomes pliable. Pressure may concurrently be applied to the sheet to form the now pliable sheet around the mold. Once the sheet cools, it will have a shape that conforms to the mold. In one embodiment, a release agent (e.g., a non-stick material) is applied to the mold before forming the shell. This may facilitate later removal of the mold from the shell. Forces may be applied to lift the appliance from the mold. In some instances, a breakage, warpage, or deformation may result from the removal forces. Accordingly, embodiments disclosed herein may determine where the probable point or points of damage may occur in a digital design of the appliance prior to manufacturing and may perform a corrective action.

After an appliance is formed over a mold for a treatment stage, the appliance is removed from the mold (e.g., automated removal of the appliance from the mold), and the appliance is subsequently trimmed along a cutline (also referred to as a trim line). The processing logic may determine a cutline for the appliance. The determination of the cutline(s) may be made based on the virtual 3D model of the dental arch at a particular treatment stage, based on a virtual 3D model of the appliance to be formed over the dental arch, or a combination of a virtual 3D model of the dental arch and a virtual 3D model of the appliance. The location and shape of the cutline can be important to the functionality of the appliance (e.g., an ability of the appliance to apply desired forces to a patient's teeth) as well as the fit and comfort of the appliance. For shells such as orthodontic appliances, orthodontic retainers and orthodontic splints, the trimming of the shell may play a role in the efficacy of the shell for its intended purpose (e.g., aligning, retaining or positioning one or more teeth of a patient) as well as the fit of the shell on a patient's dental arch. For example, if too much of the shell is trimmed, then the shell may lose rigidity and an ability of the shell to exert force on a patient's teeth may be compromised. When too much of the shell is trimmed, the shell may become weaker at that location and may be a point of damage when a patient removes the shell from their teeth or when the shell is removed from the mold. In some embodiments, the cut line may be modified in the digital design of the appliance as one of the corrective actions taken when a probable point of damage is determined to exist in the digital design of the appliance.

On the other hand, if too little of the shell is trimmed, then portions of the shell may impinge on a patient's gums and cause discomfort, swelling, and/or other dental issues. Additionally, if too little of the shell is trimmed at a location, then the shell may be too rigid at that location. In some embodiments, the cutline may be a straight line across the appliance at the gingival line, below the gingival line, or above the gingival line. In some embodiments, the cutline may be a gingival cutline that represents an interface between an appliance and a patient's gingiva. In such embodiments, the cutline controls a distance between an edge of the appliance and a gum line or gingival surface of a patient.

Each patient has a unique dental arch with unique gingiva. Accordingly, the shape and position of the cutline may be unique and customized for each patient and for each stage of treatment. For instance, the cutline is customized to follow along the gum line (also referred to as the gingival line). In some embodiments, the cutline may be away from the gum line in some regions and on the gum line in other regions. For example, it may be desirable in some instances for the cutline to be away from the gum line (e.g., not touching the gum) where the shell will touch a tooth and on the gum line (e.g., touching the gum) in the interproximal regions between teeth. Accordingly, it is important that the shell be trimmed along a predetermined cutline.

In some embodiments, the dental appliances (e.g., orthodontic appliances) herein (or portions thereof) can be produced using direct fabrication, such as additive manufacturing techniques (also referred to herein as “3D printing) or subtractive manufacturing techniques (e.g., milling). In some embodiments, direct fabrication involves forming an object (e.g., an orthodontic appliance or a portion thereof) without using a physical template (e.g., mold, mask etc.) to define the object geometry. Additive manufacturing techniques can be categorized as follows: (1) vat photopolymerization (e.g., stereolithography), in which an object is constructed layer by layer from a vat of liquid photopolymer resin; (2) material jetting, in which material is jetted onto a build platform using either a continuous or drop on demand (DOD) approach; (3) binder jetting, in which alternating layers of a build material (e.g., a powder-based material) and a binding material (e.g., a liquid binder) are deposited by a print head; (4) fused deposition modeling (FDM), in which material is drawn though a nozzle, heated, and deposited layer by layer; (5) powder bed fusion, including but not limited to direct metal laser sintering (DMLS), electron beam melting (EBM), selective heat sintering (SHS), selective laser melting (SLM), and selective laser sintering (SLS); (6) sheet lamination, including but not limited to laminated object manufacturing (LOM) and ultrasonic additive manufacturing (UAM); and (7) directed energy deposition, including but not limited to laser engineering net shaping, directed light fabrication, direct metal deposition, and 3D laser cladding. For example, stereolithography can be used to directly fabricate one or more of the appliances 712, 714, and 716. In some embodiments, stereolithography involves selective polymerization of a photosensitive resin (e.g., a photopolymer) according to a desired cross-sectional shape using light (e.g., ultraviolet light). The object geometry can be built up in a layer-by-layer fashion by sequentially polymerizing a plurality of object cross-sections. As another example, the appliances 712, 714, and 716 can be directly fabricated using selective laser sintering. In some embodiments, selective laser sintering involves using a laser beam to selectively melt and fuse a layer of powdered material according to a desired cross-sectional shape in order to build up the object geometry. As yet another example, the appliances 712, 714, and 716 can be directly fabricated by fused deposition modeling. In some embodiments, fused deposition modeling involves melting and selectively depositing a thin filament of thermoplastic polymer in a layer-by-layer manner in order to form an object. In yet another example, material jetting can be used to directly fabricate the appliances 712, 714, and 716. In some embodiments, material jetting involves jetting or extruding one or more materials onto a build surface in order to form successive layers of the object geometry.

FIG. 7C illustrates a method 750 of orthodontic treatment using a plurality of appliances, in accordance with embodiments. One or more of the plurality of appliances may be formed using modular trays and/or global plan data, as disclosed herein. The method 750 can be practiced using any of the appliances or appliance sets described herein. In block 760, a first orthodontic appliance is applied to a patient's teeth in order to reposition the teeth from a first tooth arrangement to a second tooth arrangement. In block 770, a second orthodontic appliance is applied to the patient's teeth in order to reposition the teeth from the second tooth arrangement to a third tooth arrangement. The method 750 can be repeated as necessary using any suitable number and combination of sequential appliances in order to incrementally reposition the patient's teeth from an initial arrangement to a target arrangement. The appliances can be generated all at the same stage or in sets or batches (e.g., at the beginning of a stage of the treatment), or the appliances can be fabricated one at a time, and the patient can wear each appliance until the pressure of each appliance on the teeth can no longer be felt or until the maximum amount of expressed tooth movement for that given stage has been achieved. A plurality of different appliances (e.g., a set) can be designed and even fabricated prior to the patient wearing any appliance of the plurality. After wearing an appliance for an appropriate period of time, the patient can replace the current appliance with the subsequent appliance in the series until no more appliances remain. The appliances are generally not affixed to the teeth and the patient may place and replace the appliances at any time during the procedure (e.g., patient-removable appliances). The final appliance or several appliances in the series may have a geometry or geometries selected to overcorrect the tooth arrangement. For instance, one or more appliances may have a geometry that would (if fully achieved) move individual teeth beyond the tooth arrangement that has been selected as the “final.” Such over-correction may be desirable in order to offset potential relapse after the repositioning method has been terminated (e.g., permit movement of individual teeth back toward their pre-corrected positions). Over-correction may also be beneficial to speed the rate of correction (e.g., an appliance with a geometry that is positioned beyond a desired intermediate or final position may shift the individual teeth toward the position at a greater rate). In such cases, the use of an appliance can be terminated before the teeth reach the positions defined by the appliance. Furthermore, over-correction may be deliberately applied in order to compensate for any inaccuracies or limitations of the appliance.

FIG. 8 illustrates a method 800 for designing an orthodontic appliance to be produced by direct fabrication, in accordance with embodiments. Some or all of the blocks of the method 800 can be performed by any suitable data processing system or device, e.g., one or more processors configured with suitable instructions. In some embodiments, method 800 is performed using analysis module 256 of FIG. 2A and/or AI model 458B of FIG. 4C.

In block 810, a movement path to move one or more teeth from an initial arrangement to a target arrangement is determined. The initial arrangement can be determined from a mold or a scan of the patient's teeth or mouth tissue, e.g., using wax bites, direct contact scanning, x-ray imaging, tomographic imaging, sonographic imaging, and other techniques for obtaining information about the position and structure of the teeth, jaws, gums and other orthodontically relevant tissue. From the obtained data, a digital data set can be derived that represents the initial (e.g., pretreatment) arrangement of the patient's teeth and other tissues. Optionally, the initial digital data set is processed to segment the tissue constituents from each other. For example, data structures that digitally represent individual tooth crowns can be produced. Advantageously, digital models of entire teeth can be produced, including measured or extrapolated hidden surfaces and root structures, as well as surrounding bone and soft tissue.

The target arrangement of the teeth (e.g., a desired and intended end result of orthodontic treatment) can be received from a clinician in the form of a prescription, can be calculated from basic orthodontic principles, and/or can be extrapolated computationally from a clinical prescription. With a specification of the desired final positions of the teeth and a digital representation of the teeth themselves, the final position and surface geometry of each tooth can be specified to form a complete model of the tooth arrangement at the desired end of treatment.

Having both an initial position and a target position for each tooth, a movement path can be defined for the motion of each tooth. In some embodiments, the movement paths are configured to move the teeth in the quickest fashion with the least amount of round-tripping to bring the teeth from their initial positions to their desired target positions. The tooth paths can optionally be segmented, and the segments can be calculated so that each tooth's motion within a segment stays within threshold limits of linear and rotational translation. In this way, the end points of each path segment can constitute a clinically viable repositioning, and the aggregate of segment end points can constitute a clinically viable sequence of tooth positions, so that moving from one point to the next in the sequence does not result in a collision of teeth.

In block 820, a force system to produce movement of the one or more teeth along the movement path may be determined. A force system can include one or more forces and/or one or more torques. Different force systems can result in different types of tooth movement, such as tipping, translation, rotation, extrusion, intrusion, root movement, etc. Biomechanical principles, modeling techniques, force calculation/measurement techniques, and the like, including knowledge and approaches commonly used in orthodontia, may be used to determine the appropriate force system to be applied to the tooth to accomplish the tooth movement. In determining the force system to be applied, sources may be considered including literature, force systems determined by experimentation or virtual modeling, computer-based modeling, clinical experience, minimization of unwanted forces, etc.

The determination of the force system can include constraints on the allowable forces, such as allowable directions and magnitudes, as well as desired motions to be brought about by the applied forces. For example, in fabricating palatal expanders, different movement strategies may be desired for different patients. For example, the amount of force needed to separate the palate can depend on the age of the patient, as very young patients may not have a fully-formed suture. Thus, in juvenile patients and others without fully-closed palatal sutures, palatal expansion can be accomplished with lower force magnitudes. Slower palatal movement can also aid in growing bone to fill the expanding suture. For other patients, a more rapid expansion may be desired, which can be achieved by applying larger forces. These requirements can be incorporated as needed to choose the structure and materials of appliances; for example, by choosing palatal expanders capable of applying large forces for rupturing the palatal suture and/or causing rapid expansion of the palate. Subsequent appliance stages can be designed to apply different amounts of force, such as first applying a large force to break the suture, and then applying smaller forces to keep the suture separated or gradually expand the palate and/or arch.

The determination of the force system can also include modeling of the facial structure of the patient, such as the skeletal structure of the jaw and palate. Scan data of the palate and arch, such as X-ray data or 3D optical scanning data, for example, can be used to determine parameters of the skeletal and muscular system of the patient's mouth, so as to determine forces sufficient to provide a desired expansion of the palate and/or arch. In some embodiments, the thickness and/or density of the mid-palatal suture may be measured, or input by a treating professional. In other embodiments, the treating professional can select an appropriate treatment based on physiological characteristics of the patient. For example, the properties of the palate may also be estimated based on factors such as the patient's age—for example, young juvenile patients will typically require lower forces to expand the suture than older patients, as the suture has not yet fully formed.

In block 830, appliance design for an orthodontic appliance configured to produce the force system may be determined. Determination of the orthodontic appliance, appliance geometry, material composition, and/or properties can be performed using a treatment or force application simulation environment and/or application of machine learning. A simulation environment can include, e.g., computer modeling systems, biomechanical systems or apparatus, and the like. Optionally, digital models of the appliance and/or teeth can be produced, such as finite element models. The finite element models can be created using computer program application software available from a variety of vendors. For creating solid geometry models, computer aided engineering (CAE) or computer aided design (CAD) programs can be used, such as the AutoCAD® software products available from Autodesk, Inc., of San Rafael, CA. For creating finite element models and analyzing them, program products from a number of vendors can be used, including finite element analysis packages from ANSYS, Inc., of Canonsburg, PA, and SIMULIA (Abaqus) software products from Dassault Systèmes of Waltham, MA.

Optionally, one or more orthodontic appliances can be selected for testing or force modeling. As noted above, a desired tooth movement, as well as a force system required or desired for eliciting the desired tooth movement, can be identified. Using the simulation environment, a candidate orthodontic appliance can be analyzed or modeled for determination of an actual force system resulting from use of the candidate appliance. One or more modifications can optionally be made to a candidate appliance, and force modeling can be further analyzed as described, e.g., in order to iteratively determine an appliance design that produces the desired force system.

In block 840, instructions for fabrication of the orthodontic appliance incorporating the appliance design are generated. The instructions can be configured to control a fabrication system or device in order to produce the orthodontic appliance with the specified orthodontic appliance. In some embodiments, the instructions are configured for manufacturing the orthodontic appliance using direct fabrication (e.g., stereolithography, selective laser sintering, fused deposition modeling, 3D printing, continuous direct fabrication, multi-material direct fabrication, etc.), in accordance with the various methods presented herein. In alternative embodiments, the instructions can be configured for indirect fabrication of the appliance, e.g., by thermoforming. In some embodiments, the instructions for fabrication of the orthodontic appliance include instructions for performing modular trays and/or global plan data, as disclosed herein.

Method 800 may comprise additional blocks: 1) The upper arch and palate of the patient is scanned intraorally to generate three dimensional data of the palate and upper arch; and/or 2) The three dimensional shape profile of the appliance is determined to provide a gap and teeth engagement structures.

Although the above blocks show a method 800 of designing an orthodontic appliance in accordance with some embodiments, a person of ordinary skill in the art will recognize some variations based on the teaching described herein. Some of the blocks may comprise sub-blocks. Some of the blocks may be repeated as often as desired. One or more blocks of the method 800 may be performed with any suitable fabrication system or device, such as the embodiments described herein. Some of the blocks may be optional, and the order of the blocks can be varied as desired.

FIG. 9 illustrates a method 900 for digitally planning an orthodontic treatment and/or design or fabrication of an appliance, in accordance with embodiments. The method 900 can be applied to any of the treatment procedures described herein and can be performed by any suitable data processing system.

In block 910, a digital representation of a patient's teeth is received. The digital representation can include surface topography data for the patient's intraoral cavity (including teeth, gingival tissues, etc.). The surface topography data can be generated by directly scanning the intraoral cavity, a physical model (positive or negative) of the intraoral cavity, or an impression of the intraoral cavity, using a suitable scanning device (e.g., a handheld scanner, desktop scanner, etc.).

In block 920, one or more treatment stages are generated based on the digital representation of the teeth. The treatment stages can be incremental repositioning stages of an orthodontic treatment procedure designed to move one or more of the patient's teeth from an initial tooth arrangement to a target arrangement. For example, the treatment stages can be generated by determining the initial tooth arrangement indicated by the digital representation, determining a target tooth arrangement, and determining movement paths of one or more teeth in the initial arrangement necessary to achieve the target tooth arrangement. The movement path can be optimized based on minimizing the total distance moved, preventing collisions between teeth, avoiding tooth movements that are more difficult to achieve, or any other suitable criteria.

In block 930, at least one orthodontic appliance is fabricated based on the generated treatment stages. For example, a set of appliances can be fabricated, each shaped according a tooth arrangement specified by one of the treatment stages, such that the appliances can be sequentially worn by the patient to incrementally reposition the teeth from the initial arrangement to the target arrangement. The appliance set may include one or more of the orthodontic appliances described herein. The fabrication of the appliance may involve creating a digital model of the appliance to be used as input to a computer-controlled fabrication system. The appliance can be formed using direct fabrication methods, indirect fabrication methods, or combinations thereof, as desired. The fabrication of the appliance may include modular trays and/or global plan data, as disclosed herein.

In some instances, staging of various arrangements or treatment stages may not be necessary for design and/or fabrication of an appliance. As illustrated by the dashed line in FIG. 9, design and/or fabrication of an orthodontic appliance, and perhaps a particular orthodontic treatment, may include use of a representation of the patient's teeth (e.g., receive a digital representation of the patient's teeth at block 910), followed by design and/or fabrication of an orthodontic appliance based on a representation of the patient's teeth in the arrangement represented by the received representation.

Some examples have been described with reference to orthodontic treatment plans that include a series of stages that are each associated with a different orthodontic aligner. It should be understood that any such examples described with reference to orthodontic treatment and a series of orthodontic aligners also applies to palatal expansion treatment and a series of palatal expanders. For palatal expansion treatment, a similar process may be performed as described above for orthodontic treatment. For example, an upper and/or lower dental arch and upper palate may be scanned using an intraoral scanner to generate a 3D model of the dental arch(es) and of the upper palate. A final shape (e.g., width) of the upper palate may be determined, and a series of treatment stages to progress from a current upper palate shape and a final target upper palate shape may be determined. For each treatment stage, a polymeric palatal expander may be fabricated, either via direct fabrication (e.g., direct 3D printing) or by 3D printing of a mold and thermoforming a palatal expander over the mold. Materials used for palatal expanders may be the same as or different from those used for orthodontic aligners in embodiments.

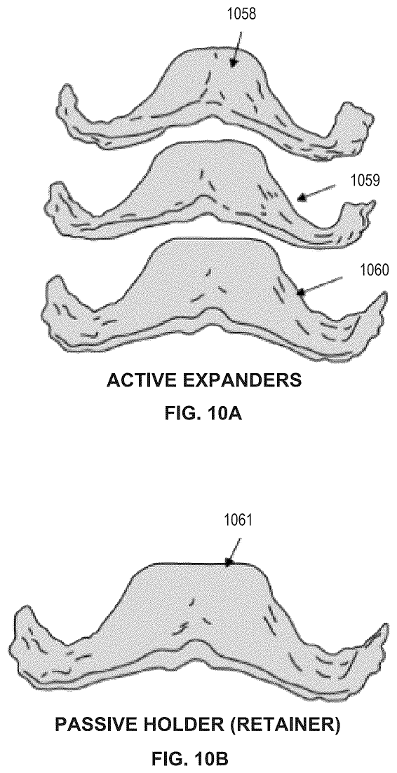

FIG. 10A shows an example of a series of palatal expanders that get progressively broader. For example, an initial upper palatal expander 1058 may have narrower palatal region than the intermediate palatal expander 1059 and a final palatal expander 1060. FIG. 10B illustrates an example of a passive holder (e.g., retainer) 1061 that may be worn after the series has completed expanding the patient's palate. In this example, the palatal expander retainer 1061 is similar or identical to the last of the palatal expanders in the sequence, although it may have a different configuration.

As mentioned above, a palatal expander as described herein can be one of a series of palatal expanders (incremental palatal expanders) that may be used to expand a subject's palate from an initial size/shape toward a target size/shape. For example, the methods and improvements described herein may be incorporated into a palatal expander or series of palatal expander as described, for example, in US20190314119A1, herein incorporated by reference in its entirety. A series of palatal expanders may be configured to expand the patient's palate by a predetermined distance (e.g., the distance between the molar regions of one expander may differ from the distance between the molar regions of the prior expander by not more than 2 mm, by between 0.1 and 2 mm, by between 0.25 and 1 mm, etc.) and/or by a predetermined force (e.g., limiting the force applied to less than 180 Newtons (N), to between 8-200 N, between 8-90 N, between 8-80 N, between 8-70 N, between 8-60 N, between 8-50 N, between 8-40 N, between 8-30 N, between 30-60 N, between 30-70 N, between 40-60 N, between 40-70 N, between 60-200 N, between 70-180 N, between 70-160 N, etc., including any range there between).

The palatal region may be between about 1-5 mm thick (e.g., between 1.5 to 3 mm, between 2 and 2.5 mm thick, etc.). The occlusal side may have a thickness of between about 0.5-2 mm (e.g., between 0.5 to 1.75 mm, between 0.75 to 1.7 mm, etc.). The buccal side may have a thickness of between about 0.25-1 mm (e.g., between 0.35 and 0.85 mm, between about 0.4 and 0.8 mm, etc.).

The dental devices described herein can include any of a number of features to facilitate the expansion process, improve patient comfort, and/or aid in insertion/retention of the dental devices in the patient's dentition. Examples of some features of dental devices are described in U.S. Patent Application Publication No. 2018/0153648A1, filed on Dec. 4, 2017, which is incorporated herein by reference in its entirety. For example, any of the dental devices described herein may include any number of attachment features that are configured to couple with corresponding attachments bonded to the patient's teeth. The dental devices may have regions of varying thickness. In any of the dental devices described herein can have varied thicknesses. For example, the thickness of a palatal region may be thicker or thinner than the thickness of tooth engagement regions. The palatal region of any of the palatal expanders may include one or more cut-out regions, which may enhance comfort and/or prevent problems with speech.

Any of the methods (including user interfaces) described herein may be implemented as software, hardware or firmware, and may be described as a non-transitory machine-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, computer models (e.g., for additive manufacturing) and instructions related to forming a dental device may be stored on a non-transitory machine-readable storage medium.

It should be understood that the above description is intended to be illustrative, and not restrictive. Many other embodiment examples will be apparent to those of skill in the art upon reading and understanding the above description. Although the present disclosure describes specific examples, it will be recognized that the systems and methods of the present disclosure are not limited to the examples described herein, but may be practiced with modifications within the scope of the appended claims. Accordingly, the specification and drawings are to be regarded in an illustrative sense rather than a restrictive sense. The scope of the present disclosure should, therefore, be determined with reference to the appended claims, along with the full scope of equivalents to which such claims are entitled.

The embodiments of methods, hardware, software, firmware, or code set forth above may be implemented via instructions or code stored on a machine-accessible, machine readable, computer accessible, or computer readable medium which are executable by a processing element. “Memory” includes any mechanism that provides (i.e., stores and/or transmits) information in a form readable by a machine, such as a computer or electronic system. For example, “memory” includes random-access memory (RAM), such as static RAM (SRAM) or dynamic RAM (DRAM); ROM; magnetic or optical storage medium; flash memory devices; electrical storage devices; optical storage devices; acoustical storage devices, and any type of tangible machine-readable medium suitable for storing or transmitting electronic instructions or information in a form readable by a machine (e.g., a computer).

Reference throughout this specification to “one embodiment” or “an embodiment” means that a particular feature, structure, or characteristic described in connection with the embodiment is included in at least one embodiment of the disclosure. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

In the foregoing specification, a detailed description has been given with reference to specific exemplary embodiments. It will, however, be evident that various modifications and changes may be made thereto without departing from the broader spirit and scope of the disclosure as set forth in the appended claims. The specification and drawings are, accordingly, to be regarded in an illustrative sense rather than a restrictive sense. Furthermore, the foregoing use of embodiment, embodiment, and/or other exemplarily language does not necessarily refer to the same embodiment or the same example, but may refer to different and distinct embodiments, as well as potentially the same embodiment.

The words “example” or “exemplary” are used herein to mean serving as an example, instance, or illustration. Any aspect or design described herein as “example’ or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or.” That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. In addition, the articles “a” and “an” as used in this application and the appended claims should generally be construed to mean “one or more” unless specified otherwise or clear from context to be directed to a singular form. Moreover, use of the term “an embodiment” or “one embodiment” or “an embodiment” or “one embodiment” throughout is not intended to mean the same embodiment or embodiment unless described as such. Also, the terms “first,” “second,” “third,” “fourth,” etc. as used herein are meant as labels to distinguish among different elements and may not necessarily have an ordinal meaning according to their numerical designation.

A digital computer program, which may also be referred to or described as a program, software, a software application, a module, a software module, a script, or code, can be written in any form of programming language, including compiled or interpreted languages, or declarative or procedural languages, and it can be deployed in any form, including as a stand-alone program or as a module, component, subroutine, or other unit suitable for use in a digital computing environment. The essential elements of a digital computer a central processing unit for performing or executing instructions and one or more memory devices for storing instructions and digital data. The central processing unit and the memory can be supplemented by, or incorporated in, special purpose logic circuitry or quantum simulators. Generally, a digital computer will also include, or be operatively coupled to receive digital data from or transfer digital data to, or both, one or more mass storage devices for storing digital data, e.g., magnetic, magneto-optical disks, optical disks, or systems suitable for storing information. However, a digital computer need not have such devices.

Digital computer-readable media suitable for storing digital computer program instructions and digital data include all forms of non-volatile digital memory, media, and memory devices, including by way of example semiconductor memory devices, e.g., EPROM, EEPROM, and flash memory devices; magnetic disks, e.g., internal hard disks or removable disks; magneto-optical disks; CD-ROM and DVD-ROM disks.

Control of the various systems described in this specification, or portions of them, can be implemented in a digital computer program product that includes instructions that are stored on one or more non-transitory machine-readable storage media, and that are executable on one or more digital processing devices. The systems described in this specification, or portions of them, can each be implemented as an apparatus, method, or system that may include one or more digital processing devices and memory to store executable instructions to perform the operations described in this specification.

The following exemplary embodiments are now described.

Embodiment 1: A method for optimizing a dental treatment plan, the method comprising: accessing a dental treatment plan for a patient comprising a series of sequential treatment stages, each treatment stage associated with a particular dental appliance in a series of dental appliances; receiving patient data comprising one or more progress indicators associated with the dental treatment plan; determining, based on the one or more progress indicators, a level of progress associated with the dental treatment plan; based at least in part on the determined level of progress, determining a treatment modification for the patient, wherein the treatment modification comprises: advancing the patient to a subsequent treatment stage in the series of sequential treatment stages before a preplanned advancement time, or retaining the patient in a treatment current stage of the series of sequential treatment stages beyond the preplanned advancement time; and generating a notification indicating the determined treatment modification.

Embodiment 2: The method of embodiment 1, wherein the notification is sent to a patient system, the notification comprising instructions to the patient to wear a subsequent dental appliance corresponding to the subsequent treatment stage or to continue to wear a current dental appliance corresponding to the current treatment stage.

Embodiment 3: The method of embodiments 1-2, wherein the notification is sent to a dental professional system associated with a health care provider (HCP).

Embodiment 4: The method of embodiments 1-3, wherein the patient data comprises at least one of image data of a dentition of the patient, biomarker data indicative of changes in the dentition, pressure data indicative of a level of pressure exerted by the dentition on a dental appliance, or data indicative of an electrical parameter associated with a position of a tooth with respect to a dental appliance.

Embodiment 5: The method of embodiments 1-4, wherein the one or more progress indicators comprises at least one of indicators of a position of a tooth or indicators of a level of movement of a tooth.

Embodiment 6: The method of embodiments 1-5, wherein processing the patient data to determine a level of progress associated with the dental treatment plan comprises causing a representation of the patient data to be presented to a healthcare provider (HCP) on a graphical user interface.

Embodiment 7: The method of embodiments 1-6, wherein processing the patient data to determine the level of progress associated with the dental treatment plan comprises providing the patient data as input to one or more machine learning models and receiving, as output from the one or more machine learning models, the level of progress associated with the dental treatment plan.

Embodiment 8: The method of embodiments 1-7, wherein processing the patient data to determine the level of progress associated with the dental treatment plan comprises comparing the one or more progress indicators of the received patient data to historical progress indicators of historical patient data.

Embodiment 9: The method of embodiments 1-8, wherein the dental treatment plan is designed based on historical patient data that is collected from a plurality of patients.

Embodiment 10: The method of embodiments 1-9, wherein the patient data is received periodically at a frequency level, the method further comprising: increasing or decreasing the frequency level based on the determined level of progress.

Embodiment 11: The method of embodiments 1-10, wherein the dental treatment plan comprises an assessment schedule comprising one or more patient assessments that are each separated by predefined time intervals, and wherein the treatment modification further comprises modifying the assessment schedule by shortening or lengthening one or more of the predefined time intervals.

Embodiment 12: A method for optimizing a dental treatment plan, the method comprising: receiving patient data comprising one or more progress indicators associated with a dental treatment plan for a patient; processing the patient data to determine a level of progression associated with the dental treatment plan based on the one or more progress indicators; modifying the dental treatment plan in response to the determined level of progression; and generating a notification of the modified dental treatment plan.

Embodiment 13: The method of embodiment 12, wherein modifying the dental treatment plan in response to the determined level of progression comprises advancing a patient associated with the patient data to a subsequent stage of the dental treatment plan or retaining the patient within a current stage of the dental treatment plan.

Embodiment 14: The method of embodiments 12-13, wherein the dental treatment plan comprises an assessment schedule comprising one or more patient assessments that are each separated by predefined time intervals, and wherein modifying the dental treatment plan in response to the determined level of progression comprises modifying the assessment schedule by shortening or lengthening one or more of the predefined time intervals.

Embodiment 15: The method of embodiments 12-14, wherein the notification is sent to a patient system, the notification comprising instructions to the patient to either wear a subsequent dental appliance corresponding to a subsequent stage of the dental treatment plan or continue with a current dental appliance.

Embodiment 16: The method of embodiments 12-15, wherein the notification is sent to a dental professional system associated with a health care provider (HCP) treating the patient.

Embodiment 17: The method of embodiments 12-16, wherein the patient data comprises at least one of image data of a patient's dentition, biomarker data indicative of changes in the patient's dentition, pressure data indicative of a level of pressure exerted by the patient's dentition on a dental appliance, or data indicative of an electrical parameter associated with a position of a tooth with respect to the dental appliance.

Embodiment 18: The method of embodiments 12-17, wherein the one or more progress indicators comprises at least one of indicators of a position of a tooth or indicators of a level of movement of a tooth.

Embodiment 19: The method of embodiments 12-18, wherein processing the patient data to determine a level of progression associated with the dental treatment plan comprises causing a representation of the patient data to be presented to a healthcare provider (HCP) on a graphical user interface.

Embodiment 20: The method of embodiments 12-19, wherein processing the patient data to determine a level of progression associated with the dental treatment plan comprises providing the patient data as input to one or more artificial intelligence (AI) models and receiving, as output from the one or more AI models, a level of progression associated with the dental treatment plan.

Embodiment 21: The method of embodiments 12-20, wherein processing the patient data to determine a level of progression associated with the dental treatment plan comprises comparing the one or more progress indicators of the received patient data to historical progress indicators of historical patient data.

Embodiment 22: The method of embodiments 12-21, wherein the dental treatment plan is designed based on historical patient data that is collected from a plurality of patients.

Embodiment 23: The method of embodiments 12-22, wherein the patient data is received periodically at a frequency level, the method further comprising: increasing or decreasing the frequency level based on the determined level of progression.

Embodiment 24: A method for optimizing a dental treatment plan, the method comprising:
receiving a plurality of patient data, wherein the plurality of patient data is discretized into data segments associated with individual patients and corresponding individual dental treatment plans for the individual patients, one or more of the data segments comprising at least one of a three-dimensional (3D) model of a dental arch or a two-dimensional (2D) image of the dental arch; receiving a plurality of dental treatment plan modifications, wherein each dental treatment plan modification of the plurality of dental treatment plan modifications corresponds to a given data segment of the plurality of patient data that is associated with an individual patient and the corresponding individual dental treatment plan for the individual patient; generating a training dataset comprising: the received plurality of patient data; and the received plurality of dental treatment plan modifications; and training a machine learning model using the training dataset to generate a trained machine learning model trained to output at least one of one or more new dental treatment plans or modifications to one or more existing dental treatment plans based on an input comprising at least one of a 3D model of a new dental arch or a 2D image of the new dental arch.

Embodiment 25: The method of embodiment 24, wherein training the machine learning model using the training dataset to generate one or more dental treatment plan modifications based on a data segment of patient data comprises: processing, using the machine learning model, a given data segment of the plurality of patient data within the training dataset to generate a predicted dental treatment plan modification for the dental treatment plan corresponding to the given data segment; identifying a difference between the predicted dental treatment plan modification and a correct corresponding dental treatment plan modification within the training dataset; and changing one or more parameters of the AI model to reduce or eliminate the identified difference.

Embodiment 26: The method of embodiments 24-25, further comprising: receiving additional patient data associated with an individual patient and a corresponding individual dental treatment plan for the individual patient; providing the additional patient data as input to the trained machine learning model; receiving, as output from the trained machine learning model, one or more modifications for the individual dental treatment plan; modifying the individual dental treatment plan based on the one or more modifications received from the trained machine learning model; and generating a notification of the modified individual dental treatment plan.

Embodiment 27: The method of embodiment 26, wherein modifying the individual dental treatment plan based on the one or more modifications received from the trained machine learning model comprises advancing a patient associated with the patient data to a subsequent stage of the individual dental treatment plan or retaining the patient within a current stage of the individual dental treatment plan.

Embodiment 28: The method of embodiments 26-27, wherein the individual dental treatment plan comprises an assessment schedule comprising one or more patient assessments that are each separated by predefined time intervals, and wherein modifying the individual dental treatment plan comprises modifying the assessment schedule by shortening or lengthening one or more of the predefined time intervals.

Embodiment 29: The method of embodiments 26-28, wherein the notification is sent to a patient system, the notification comprising instructions to the individual patient to either wear a subsequent dental appliance corresponding to a subsequent stage of the individual dental treatment plan or continue with a current dental appliance.

Embodiment 30: The method of embodiments 26-29, wherein the notification is sent to a dental professional system associated with a health care provider (HCP) treating the individual patient.

Embodiment 31: The method of embodiments 26-30, wherein the patient data comprises at least one of image data of a patient's dentition, biomarker data indicative of changes in the patient's dentition, pressure data indicative of a level of pressure exerted by the patient's dentition on a dental appliance, or data indicative of an electrical parameter associated with a position of a tooth with respect to a dental appliance.

Embodiment 32: The method of embodiments 26-31, wherein the dental treatment plan is designed based on historical patient data that is collected from a plurality of patients.

Embodiment 33: The method of embodiments 26-32, wherein the patient data is received periodically at a frequency level, the method further comprising: increasing or decreasing the frequency level.

Embodiment 34: A method comprising: accessing a dental treatment plan for a patient comprising a series of sequential treatment stages, each treatment stage associated with a particular dental appliance in a series of dental appliances; receiving patient data associated with one or more stages of the dental treatment plan; processing the patient data to identify one or more progress indicators of treatment progress with respect to the dental treatment plan; determining, based on the one or more progress indicators, a level of progress associated with the dental treatment plan; and performing one or more actions based at least in part on the determined level of progress.

Embodiment 35: The method of embodiment 34, wherein performing the one or more actions comprises determining a treatment modification to the dental treatment plan, wherein the treatment modification comprises: advancing the patient to a subsequent treatment stage in the series of sequential treatment stages before a preplanned advancement time, or retaining the patient in a current treatment stage of the series of sequential treatment stages beyond the preplanned advancement time.

Embodiment 36: The method of embodiment 35, wherein performing the one or more actions comprises: generating a notification indicating the determined treatment modification.

Embodiment 37: The method of embodiment 36, wherein the notification is sent to a patient system, the notification comprising instructions to the patient to wear a subsequent dental appliance corresponding to the subsequent treatment stage or to continue to wear a current dental appliance corresponding to the current treatment stage.

Embodiment 38: The method of embodiments 36-37, wherein the notification is sent to a dental professional system associated with a health care provider (HCP).

Embodiment 39: The method of embodiments 36-38, wherein the dental treatment plan comprises a combined palatal expansion and orthodontic treatment plan, and wherein advancing the patient to the subsequent treatment stage comprises advancing from a palatal expansion treatment stage to an orthodontic treatment stage.

Embodiment 40: The method of embodiment 39, wherein the palatal expansion treatment stage is implemented using a palatal expander of a series of palatal expanders or a retainer, and wherein the orthodontic treatment stage is implemented using an orthodontic aligner of a series of orthodontic aligners.

Embodiment 41: The method of embodiments 36-40, wherein the dental treatment plan comprises a combined palatal expansion and orthodontic treatment plan, wherein the series of treatment stages comprise a series of palatal expansion treatment stages followed by a series of orthodontic treatment stages, and wherein retaining the patient in the current treatment stage beyond the preplanned advancement time comprises postponing advancement from palatal expansion treatment to orthodontic treatment.

Embodiment 42: The method of embodiments 34-41, wherein the patient data comprises at least one of image data of a dentition of the patient, biomarker data indicative of changes in the dentition, pressure data indicative of a level of pressure exerted by the dentition on a dental appliance, or data indicative of an electrical parameter associated with a position of a tooth with respect to a dental appliance.

Embodiment 43: The method of embodiments 34-42, wherein the one or more progress indicators comprises at least one of indicators of a position of a tooth or indicators of a level of movement of a tooth.

Embodiment 44: The method of embodiments 34-43, further comprising: causing a representation of the patient data to be presented to a healthcare provider (HCP) on a graphical user interface.

Embodiment 45: The method of embodiments 34-44, wherein at least one of a) identifying the one or more progress indicators of treatment progress with respect to the dental treatment plan or b) determining the level of progress associated with the dental treatment plan is performed by processing the patient data using one or more machine learning models that output at least one of the one or more progress indicators or the level of progress associated with the dental treatment plan.

Embodiment 46: The method of embodiments 34-45, wherein determining the level of progress associated with the dental treatment plan comprises comparing the one or more progress indicators of the received patient data to historical progress indicators of historical patient data.

Embodiment 47: The method of embodiments 34-46, wherein the dental treatment plan is designed based on historical patient data that is collected from a plurality of patients.

Embodiment 48: The method of embodiments 34-47, wherein the patient data is received periodically at a frequency level, the method further comprising: increasing or decreasing the frequency level based on the determined level of progress associated with the dental treatment plan.

Embodiment 49: The method of embodiments 34-48, wherein the dental treatment plan comprises an assessment schedule comprising one or more patient assessments that are each separated by predefined time intervals, and wherein performing the one or more actions comprises modifying the assessment schedule by shortening or lengthening one or more of the predefined time intervals.

Embodiment 50: The method of embodiments 34-49, wherein the dental treatment plan comprises a palatal expansion treatment plan, an orthodontic treatment plan, or a combined palatal expansion and orthodontic treatment plan.

Embodiment 51: The method of embodiments 34-50, further comprising: processing the patient data to identify one or more oral health conditions of the patient; wherein the one or more actions are performed based at least in part on the one or more oral health conditions.

Embodiment 52: The method of embodiment 51, wherein performing the one or more actions comprise determining one or more treatment modifications to the dental treatment plan.

Embodiment 53: The method of embodiments 51-52, wherein performing the one or more actions comprise retaining the patient in a current treatment stage of the series of sequential treatment stages beyond a preplanned advancement time.

Embodiment 54: The method of embodiment 53, wherein the one or more oral health conditions comprise gingival inflammation.

Embodiment 55: The method of embodiments 51-54, wherein the patient data comprises at least one of a three-dimensional (3D) point cloud or a two-dimensional (2D) image of a dentition of the patient, and wherein processing the patient data to identify one or more oral health conditions comprises processing at least one of the 3D point cloud or the 2D image using a trained machine learning model, wherein the trained machine learning model outputs an identification of the one or more oral health conditions of the patient.

Embodiment 56: The method of embodiments 51-55, wherein the one or more oral health conditions comprise at least one of gingival inflammation, a tooth crack, a missing tooth, a chipped tooth, or caries.

Embodiment 57: The method of embodiments 51-56, wherein performing the one or more actions comprise outputting a recommendation to pause execution of the dental treatment plan while the one or more oral health conditions are addressed.

Embodiment 58: The method of embodiments 51-57, wherein performing the one or more actions comprises: determining one or more oral healthcare activities likely to ameliorate the one or more oral health conditions; and outputting a recommendation for the patient to adjust an oral healthcare routine to include the one or more oral healthcare activities to address the one or more oral health conditions.

Embodiment 59: The method of embodiments 51-58, wherein performing the one or more actions comprises scheduling a doctor visit.

Embodiment 60: The method of embodiments 51-59, wherein performing the one or more actions comprises: outputting a notice of the one or more oral health conditions to a device of a doctor; receiving doctor instructions associated with the one or more oral health conditions; and sending the doctor instructions to a device of the patient.

Embodiment 61: The method of embodiment 60, wherein the doctor instructions comprise instructions for the patient to perform an in-person visit with the doctor, the method further comprising: scheduling the in-person patient visit with the doctor.

Embodiment 62: The method of embodiments 51-61, further comprising: determining a severity level of each of the one or more oral health conditions, wherein the one or more actions are performed based at least in part on the severity level of the one or more oral health conditions.

Embodiment 63: The method of embodiment 62, further comprising: receiving input from a doctor indicating at least one of oral health conditions or oral health condition severity levels that warrant an in-person patient visit; and determining whether to schedule the in-person patient visit based on at least one of the one or more oral health conditions or the respective oral health condition severity levels.

Embodiment 64: The method of embodiments 51-63, wherein performing the one or more actions comprise outputting a recommendation to perform restorative treatment to treat the one or more oral health conditions in parallel to execution of the dental treatment plan.

Embodiment 65: The method of embodiments 34-64, wherein the dental treatment plan calls for dental auxiliaries on one or more teeth of the patient, and wherein the patient data comprises at least one of a three-dimensional (3D) point cloud or a two-dimensional (2D) image of a dentition of the patient, the method further comprising: identifying a missing dental auxiliary based on processing at least one of the 3D point cloud or the 2D image of the dentition of the patient; determining whether the missing dental auxiliary has clinical significance; and performing the one or more actions based at least in part on whether the missing dental auxiliary has clinical significance.

Embodiment 66: The method of embodiment 65, wherein the missing dental auxiliary has clinical significance if one or more tooth movements achieved using forces facilitated by the missing dental auxiliary have not yet been performed.

Embodiment 67: The method of embodiments 65-66, wherein the one or more actions comprise at least one of scheduling an in-person patient visit with a doctor to replace the missing dental auxiliary or slowing down treatment and are performed responsive to determining that the missing dental auxiliary has clinical significance.

Embodiment 68: The method of embodiments 34-67, wherein the dental treatment plan calls for dental auxiliaries on one or more teeth of the patient, the method further comprising: identify a missing dental auxiliary based on processing the patient data; and outputting a notice of the missing dental auxiliary to a device of a doctor.

Embodiment 69: The method of embodiments 34-68, wherein a first subset of dental appliances in the series of dental appliances was manufactured at a first time, and wherein performing the one or more actions comprises determining whether to order a second subset of dental appliances in the series of dental appliances.

Embodiment 70: The method of embodiment 69, wherein performing the one or more actions further comprises making one or more modifications to one or more dental appliances in the second subset of dental appliances in view of the level of progress prior to manufacture of the second subset of dental appliances.

Embodiment 71: The method of embodiments 34-70, wherein a current dental appliance of the series of dental appliances comprises a retainer, and wherein the patient data comprises at least one of a three-dimensional (3D) point cloud or a two-dimensional (2D) image of the retainer worn by the patient, the method further comprising: determining a level of wear of the retainer based on processing at least one of the 3D point cloud or the 2D image; determining whether the level of wear of the retainer exceeds a wear threshold; and ordering a new retainer responsive to determining that the level of wear exceeds the wear threshold.

Embodiment 72: The method of embodiments 34-71, wherein a current dental appliance of the series of dental appliances comprises a retainer, and wherein the patient data comprises at least one of a three-dimensional (3D) point cloud or a two-dimensional (2D) image of a dentition of the patient, the method further comprising: determining that one or more teeth of the patient have relapsed based on processing of at least one of the 3D point cloud or the 2D image; designing a new retainer in view of the one or more teeth of the patient that have relapsed; and ordering the new retainer.

Embodiment 73: The method of embodiments 34-72, wherein a current dental appliance of the series of dental appliances comprises a retainer, and wherein the patient data comprises at least one of a three-dimensional (3D) point cloud or a two-dimensional (2D) image of a dentition of the patient, the method further comprising: determining that one or more teeth of the patient have relapsed based on processing of at least one of the 3D point cloud or the 2D image; and updating the dental treatment plan by adding one or more orthodontic treatment stages and generating orthodontic aligners associated with the one or more orthodontic treatment stages.

Embodiment 74: The method of embodiments 34-73, wherein the dental treatment plan comprises an assessment schedule comprising one or more patient assessments that are each separated by predefined time intervals, the method further comprising: determining that a threshold number of prior patient assessments indicated that treatment was progressing as planned or faster than planned; determining that a next treatment stage is not a complex treatment stage; and shortening at least one of a current treatment stage or the next treatment stage.

Embodiment 75: The method of embodiments 34-74, further comprising: determining at least one of an initial treatment length or an initial number of treatment stages associated with the dental treatment plan; determining at least one of an updated treatment length or an updated number of treatment stages based on one or more changes to the dental treatment plan implemented during execution of the dental treatment plan; and outputting a visualization comparing at least one of the initial treatment length to the updated treatment length or the initial number of treatment stages to the updated number of treatment stages.

Embodiment 76: The method of embodiment 75, further comprising: generating simulated images of predicted future patient smiles at one or more future time periods based on one or more images of a current patient smile and at least one of the dental treatment plan or the changes to the dental treatment plan; and outputting one or more of the simulated images.

Embodiment 77: The method of embodiments 34-76, further comprising: outputting a comparison of a treatment length of the dental treatment plan of the patient as updated during treatment to an average treatment length for patients having a similar staring malocclusion as the patient.

Embodiment 78: The method of embodiments 34-77, further comprising: determining that a next treatment stage of the series of sequential treatment stages comprises a challenging tooth movement for one or more teeth; and outputting a notice for the patient to perform at least one of the following: a) be diligent in wearing the dental appliance for the next treatment stage; or b) chew on one or more chewable objects using the one or more teeth after seating the dental appliance on a dental arch of the patient to secure the dental appliance to the one or more teeth.

Embodiment 79: A system for optimizing a dental treatment plan comprising: a memory device; and a processing device communicatively coupled to the memory device, wherein the processing device is to perform the method of any of embodiments 1-78.

Embodiment 80: A non-transitory computer readable storage medium comprising instructions that, when executed by a processing device, causes the processing device to perform the method of any of embodiments 1-78.

While this specification contains many specific embodiment details, these should not be construed as limitations on the scope of what may be claimed, but rather as descriptions of features that may be specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or variation of a sub-combination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system modules and components in the embodiments described above should not be understood as requiring such separation in all embodiments, and it should be understood that the described program components and systems can generally be integrated together in a single software product or packaged into multiple software products.

Particular embodiments of the subject matter have been described. Other embodiments are within the scope of the following claims. For example, the actions recited in the claims can be performed in a different order and still achieve desirable results. As one example, the processes depicted in the accompanying figures do not necessarily require the particular order shown, or sequential order, to achieve desirable results. In some cases, multitasking and parallel processing may be advantageous.

What is claimed is:

1. A method for optimizing a dental treatment plan, the method comprising:
accessing a dental treatment plan for a patient comprising a series of sequential treatment stages, each treatment stage associated with a particular dental appliance in a series of dental appliances;
receiving patient data comprising one or more progress indicators associated with the dental treatment plan determining, based on the one or more progress indicators, a level of progress associated with the dental treatment plan;
based at least in part on the determined level of progress, determining a treatment modification for the patient, wherein the treatment modification comprises:
advancing the patient to a subsequent treatment stage in the series of sequential treatment stages before a preplanned advancement time, or
retaining the patient in a treatment current stage of the series of sequential treatment stages beyond the preplanned advancement time; and
generating a notification indicating the determined treatment modification.

2. The method of claim 1, wherein the notification is sent to a patient system, the notification comprising instructions to the patient to wear a subsequent dental appliance corresponding to the subsequent treatment stage or to continue to wear a current dental appliance corresponding to the current treatment stage.

3. The method of claim 1, wherein the patient data comprises at least one of image data of a dentition of the patient, biomarker data indicative of changes in the dentition, pressure data indicative of a level of pressure exerted by the dentition on a dental appliance, or data indicative of an electrical parameter associated with a position of a tooth with respect to a dental appliance.

4. The method of claim 1, wherein the one or more progress indicators comprises at least one of indicators of a position of a tooth or indicators of a level of movement of a tooth.

5. The method of claim 1, wherein processing the patient data to determine the level of progress associated with the dental treatment plan comprises providing the patient data as input to one or more machine learning models and receiving, as output from the one or more machine learning models, the level of progress associated with the dental treatment plan.

6. The method of claim 1, wherein processing the patient data to determine the level of progress associated with the dental treatment plan comprises comparing the one or more progress indicators of the received patient data to historical progress indicators of historical patient data.

7. The method of claim 1, wherein the dental treatment plan comprises an assessment schedule comprising one or more patient assessments that are each separated by predefined time intervals, and wherein the treatment modification further comprises modifying the assessment schedule by shortening or lengthening one or more of the predefined time intervals.

8. A method for optimizing a dental treatment plan, the method comprising:
receiving patient data comprising one or more progress indicators associated with a dental treatment plan for a patient;
processing the patient data to determine a level of progression associated with the dental treatment plan based on the one or more progress indicators;
modifying the dental treatment plan in response to the determined level of progression; and
generating a notification of the modified dental treatment plan.

9. The method of claim 8, wherein modifying the dental treatment plan in response to the determined level of progression comprises advancing a patient associated with the patient data to a subsequent stage of the dental treatment plan or retaining the patient within a current stage of the dental treatment plan.

10. The method of claim 8, wherein the dental treatment plan comprises an assessment schedule comprising one or more patient assessments that are each separated by predefined time intervals, and wherein modifying the dental treatment plan in response to the determined level of progression comprises modifying the assessment schedule by shortening or lengthening one or more of the predefined time intervals.

11. A method comprising:
accessing a dental treatment plan for a patient comprising a series of sequential treatment stages, each treatment stage associated with a particular dental appliance in a series of dental appliances;
receiving patient data associated with one or more stages of the dental treatment plan;
processing the patient data to identify one or more progress indicators of treatment progress with respect to the dental treatment plan;
determining, based on the one or more progress indicators, a level of progress associated with the dental treatment plan; and
performing one or more actions based at least in part on the determined level of progress.

12. The method of claim 11, wherein performing the one or more actions comprises determining a treatment modification to the dental treatment plan, wherein the treatment modification comprises:
advancing the patient to a subsequent treatment stage in the series of sequential treatment stages before a preplanned advancement time, or
retaining the patient in a current treatment stage of the series of sequential treatment stages beyond the preplanned advancement time.

13. The method of claim 12, wherein performing the one or more actions comprises:
generating a notification indicating the determined treatment modification, wherein the notification is sent to a patient system, the notification comprising instructions to the patient to wear a subsequent dental appliance corresponding to the subsequent treatment stage or to continue to wear a current dental appliance corresponding to the current treatment stage.

14. The method of claim 12, generating a notification indicating the determined treatment modification, wherein the notification is sent to a dental professional system associated with a health care provider, wherein the dental treatment plan comprises a combined palatal expansion and orthodontic treatment plan, and wherein advancing the patient to the subsequent treatment stage comprises advancing from a palatal expansion treatment stage to an orthodontic treatment stage.

15. The method of claim 11, wherein:
the patient data comprises at least one of image data of a dentition of the patient, biomarker data indicative of changes in the dentition, pressure data indicative of a level of pressure exerted by the dentition on a dental appliance, or data indicative of an electrical parameter associated with a position of a tooth with respect to a dental appliance; and
the one or more progress indicators comprises at least one of indicators of a position of a tooth or indicators of a level of movement of a tooth.

16. The method of claim 11, wherein at least one of a) identifying the one or more progress indicators of treatment progress with respect to the dental treatment plan or b) determining the level of progress associated with the dental treatment plan is performed by processing the patient data using one or more machine learning models that output at least one of the one or more progress indicators or the level of progress associated with the dental treatment plan.

17. The method of claim 11, wherein determining the level of progress associated with the dental treatment plan comprises comparing the one or more progress indicators of the received patient data to historical progress indicators of historical patient data.

18. The method of claim 11, further comprising:
processing the patient data to identify one or more oral health conditions of the patient;
wherein the one or more actions are performed based at least in part on the one or more oral health conditions.

19. The method of claim 11, wherein the dental treatment plan calls for dental auxiliaries on one or more teeth of the patient, and wherein the patient data comprises at least one of a three-dimensional (3D) point cloud or a two-dimensional (2D) image of a dentition of the patient, the method further comprising:
identify a missing dental auxiliary based on processing at least one of the 3D point cloud or the 2D image of the dentition of the patient;
determining whether the missing dental auxiliary has clinical significance; and
performing the one or more actions based at least in part on whether the missing dental auxiliary has clinical significance.

20. The method of claim 11, wherein a first subset of dental appliances in the series of dental appliances was manufactured at a first time, and wherein performing the one or more actions comprises determining whether to order a second subset of dental appliances in the series of dental appliances.
